(19) United States
(12) Patent Application Publication
(10) Pub. No.: US 2023/0414655 A1
(43) Pub. Date: Dec. 28, 2023

(54) COMPOSITIONS AND METHODS FOR TREATING AND PREVENTING VENOM RELATED POISONING

(71) Applicant: Arizona Board of Regents on Behalf of the University of Arizona, Tucson, AZ (US)

(72) Inventor: Vance G. Nielsen, Tucson, AZ (US)

(21) Appl. No.: 18/209,618

(22) Filed: Jun. 14, 2023

Related U.S. Application Data
(63) Continuation-in-part of application No. PCT/US2021/063846, filed on Dec. 16, 2021.
(60) Provisional application No. 63/174,085, filed on Apr. 13, 2021.
(60) Provisional application No. 63/173,869, filed on Apr. 12, 2021.
(60) Provisional application No. 63/126,380, filed on Dec. 16, 2020.

(51) Int. Cl. A61K 33/24; A61K 45/06; A61P 39/02

(52) U.S. Cl. CPC A61K 33/24; A61P 39/02; A61K 45/06

(57) ABSTRACT

This invention relates to compositions and methods for treating, ameliorating, and preventing the toxic effects of venom poisoning. In particular, the invention provides compositions comprising one or more ruthenim based-agents for one or more of inhibiting venom related procoagulant activity, inhibiting venom related phospholipase A₂ (PLA₂), and/or inhibiting venom related thrombus generation, and related methods for treating, ameliorating and preventing the toxic effects of venom poisoning in a subject suffering from or at risk of suffering from venom poisoning.

### CROSS-REFERENCE TO RELATED APPLICATIONS

The present application is a Continuation of International Application No. PCT/US2021/063846, filed Dec. 16, 2021, which claims priority to U.S. Provisional Patent Application No. 63/173,869, filed Apr. 12, 2021, U.S. Provisional Patent Application No. 63/174,085, filed Apr. 13, 2021, and U.S. Provisional Patent Application No. 63/126,380, filed Dec. 16, 2020, which are hereby incorporated by reference in their entireties.

### FIELD OF THE INVENTION

This invention relates to compositions and methods for treating, ameliorating, and preventing the toxic effects of venom poisoning. In particular, the invention provides compositions comprising one or more ruthenim based-agents for one or more of inhibiting venom related procoagulant activity, inhibiting venom related phospholipase A₂ (PLA₂), and/or inhibiting venom related thrombus generation, and related methods for treating, ameliorating and preventing the toxic effects of venom poisoning in a subject suffering from or at risk of suffering from venom poisoning.

### INTRODUCTION

In the Animal kingdom, a number of venomous animals, such as snakes, produce venom that is harmful to humans, and to their pets and livestock. For humans alone, approximately one million people throughout the world are bitten each year by venomous (poisonous) snakes. It has been estimated that of these some 100,000 die and that another 300,000 will suffer some form of disability for the remainder of their lives.

Improved methods for treating, ameliorating and preventing the toxic effects of venom poisoning are needed.

### SUMMARY OF THE INVENTION

Research has recently focused on quantifying the effects of scores of snake venoms (see, Nielsen V G, Bazzell C M (2017) J Thromb Thrombolysis 43, 203-208; Nielsen V G, Frank N, Matika R W (2018) Biometals 31, 51-59; Nielsen V G, Frank N (2019) Hum Exp Toxicol 38, 216-226; Nielsen V G, Frank N, Afshar S (2019) Toxins (Basel) 11, E94) and anticoagulant enzymes isolated from such venoms (see, Suntravat M, et al., Biometals 31, 585-593; Nielsen V G (2019) J Thromb Thrombolysis 47, 73-79) on human plasmatic coagulation, with an emphasis on the inhibitory action of carbon monoxide (CO) on such anticoagulant activity. The source of site-directed CO application to these venoms in isolation prior to placement into human plasma was release from a ruthenium (Ru)-based carbon monoxide releasing molecule (tricarbonyldichlororuthenium(II) dimer (CORM-2)). The specificity of CO mediated effect was by concurrent exposure of venom to an inactive releasing molecule that had undergone a degradation (see, Nielsen V G, Bazzell C M (2017) J Thromb Thrombolysis 43, 203-208; Nielsen V G, Frank N, Matika R W (2018) Biometals 31, 51-59; Nielsen V G, Frank N (2019) Hum Exp Toxicol 38, 216-226; Nielsen V G, Frank N, Afshar S (2019) Toxins (Basel) 11, E94; Nielsen V G (2019) J Thromb Thrombolysis 47, 73-79) with the result that this molecule would not inhibit venom activity to the extent that CORM-2 had inhibited activity (see, Nielsen V G, Bazzell C M (2017) J Thromb Thrombolysis 43, 203-208; Nielsen V G, Frank N, Matika R W (2018) Biometals 31, 51-59; Nielsen V G, Frank N (2019) Hum Exp Toxicol 38, 216-226; Nielsen V G, Frank N, Afshar S (2019) Toxins (Basel) 11, E94; Nielsen V G (2019) J Thromb Thrombolysis 47, 73-79). This paradigm is decades old, but it was challenged in the setting of K⁺ channel inhibition (see, Gessner G, et al., Eur J Pharmacol 815, 33-41) and antibacterial activity (see, Southam H M, et al., Redox Biol 18, 114-123) exerted by Ru(II) based CORM. These recent investigations determined that these Ru(II) CORM formed a transition state that, after releasing CO, would bind to histidine (see, Gessner G, et al., Eur J Pharmacol 815, 33-41; Southam H M, et al., Redox Biol 18, 114-123), methionine (see, Southam H M, et al., Redox Biol 18, 114-123), glutathione (see, Southam H M, et al., Redox Biol 18, 114-123), or cysteine (see, Southam H M, et al., Redox Biol 18, 114-123). In response to these new findings (see, Gessner G, et al., Eur J Pharmacol 815, 33-41; Southam H M, et al., Redox Biol 18, 114-123), it was first reported that purified phospholipase A₂ isolated from bee venom was inhibited by CORM-2 via a CO-independent mechanism (see, Nielsen V G (2020) J Thromb Thrombolysis 49, 100-107). Subsequently, anticoagulant metalloproteinase activity in venoms collected from mambas was found to be inhibited by CORM-2 by a similar mechanism (see, Nielsen V G, et al., Int J Mol Sci 21, 2082), and, finally, the procoagulant activity exerted by metalloproteinases and serine proteases was found to be inhibited by CORM-2 and ruthenium(III) chloride (RuCl₃) (see, Nielsen V G (2020) Int J Mol Sci 21, 2970). Thus, as suggested by recent works (see, Nielsen V G (2020) J Thromb Thrombolysis 49, 100-107; Nielsen V G, et al., Int J Mol Sci 21, 2082; Nielsen V G (2020) Int J Mol Sci 21, 2970), it is likely that ruthenium species, not CO, are binding to key anticoagulant/procoagulant venom enzymes in a heme-independent and perhaps irreversible fashion.

Of interest, multiple Ru-based molecular species have been synthesized and investigated as potential chemotherapeutic agents to replace the toxic platinum-based medications (e.g., cisplatin, carboplatin) to treat various cancers (see, Lazić D, et al., Dalton Trans 45, 4633; Hanif M, et al., ChemPlusChem 82, 841-847; Stanic-Vucinic D, et al., (2020) J Biol Inorg Chem 25, 253-265; Yocom K M, et al., (1982) Proc Natl Acad Sci USA 79, 7052-7055; Kratz K, et al., (1994) Met Based Drugs 1, 169-173; Webb M I, Walsby C J (2015) Dalton Trans 44, 17482; Ren C, Bobst C E, Kaltashov I A (2019) et al., Anal Chem 91, 7189-7198; Das D, et al., J Phys Chem B 124, 6459-6474). Thus, investigations have demonstrated that Ru(II) based compounds covalently bind to histidine, methionine, glutathione, or cysteine (see, Southam H M, et al., (2018) Redox Biol 18, 114-123; Lazić D, et al., (2016) Dalton Trans 45, 4633; Hanif M, et al., (2017) ChemPlusChem 82, 841-847; Stanic-Vucinic D, et al., (2020) J Biol Inorg Chem 25, 253-265), and Ru(III) based compounds similarly bind histidine and cysteine (see, Yocom K M, et al., (1982) Proc Natl Acad Sci USA 79, 7052-7055; Kratz K, et al., (1994) Met Based Drugs 1, 169-173; Webb M I, Walsby C J (2015) Dalton Trans 44, 17482; Ren C, Bobst C E, Kaltashov I A (2019) et al., Anal Chem 91, 7189-7198; Das D, et al., J Phys Chem B 124, 6459-6474). These binding characteristics of Ru compounds to specific amino acid residues may explain why CORM-2 and RuCl₃ separately have inhibited snake venom and isolated enzyme activities (see, Nielsen V G, Bazzell C M (2017) J Thromb Thrombolysis 43, 203-208; Nielsen V G, Frank N, Matika R W (2018) Biometals 31, 51-59; Nielsen V G, Frank N (2019) Hum Exp Toxicol 38, 216-226; Nielsen V G, Frank N, Afshar S (2019) Toxins (Basel) 11, E94; Suntravat M, et al., (2018) Biometals 31, 585-593; Nielsen V G (2019) J Thromb Thrombolysis 47, 73-79; Nielsen V G (2020) et al., J Thromb Thrombolysis 49, 100-107; Nielsen V G, Wagner M T, Frank N (2020) Int J Mol Sci 21, 2082; Nielsen V G (2020) Int J Mol Sci 21, 2970) as highly conserved histidines and disulfide bridges that are critical to function are found in snake venom metalloproteinases (SVMP) (see, Watanabe L, et al., (2003) Protein Sci 12, 2273-2281; Markland Jr. F S, Swenson S (2013) Toxicon 62, 3-18), snake venom serine proteases (SVSP) (see, Calvete J J, et al., (1997) FEBS Lett 416, 197-202; Braud S, et al., (2000) J Biol Chem 2000 275, 1823-1828) and phospholipase A₂ (PLA₂) (see, Valentin E, Lambeau G (2000) Biochimie 82, 815-831). Taken as a whole, small molecular weight, Ru based compounds may inhibit anticoagulant/procoagulant snake venom activity by binding to a hereto unappreciated Achilles heel of highly conserved amino acid residues essential to function shared across multiple enzyme types.

However, the inhibitory effects of any class of compound are not just based on valance, but also on size, composition, and other characteristics that can change the affinity to a ligand. The structures of CORM-2, CORM-3, RuCl₃ and carboplatin are displayed in FIG. 1 with their respective valence indicated. Of interest, CORM-2 and RuCl₃ have been found to have similar or no inhibitory effects on various procoagulant venoms when tested separately (see, Nielsen V G (2020) Int J Mol Sci 21, 2970). This finding opened the possibility that the Ru-based and Pt-based compounds may bind to the same critical amino acid residue with perhaps different affinity, to different residues that are enzymatically important, or perhaps to more than two molecular sites on any given enzyme. It should also be noted that the proteome of such venoms contains a great deal of similar or diverse enzymes with different effects on coagulation that summate into primarily anticoagulant or procoagulant activities (see, Nielsen V G, Bazzell C M (2017) J Thromb Thrombolysis 43, 203-208; Nielsen V G, Frank N, Matika R W (2018) Biometals 31, 51-59; Nielsen V G, Frank N (2019) Hum Exp Toxicol 38, 216-226; Nielsen V G, Frank N, Afshar S (2019) Toxins (Basel) 11, E94).

Thus, given the aforementioned molecular complexity, experiments conducted during the course of developing embodiments for the present invention (see, Examples I, II and IV) sought to determine the effects of CORM-2 and RuCl₃ exposure (separately and as a formulation) on a variety of diverse procoagulant snake venoms to provide insight into any interactions of the compounds on venom procoagulant activity. Utilizing venom collected from four diverse genera (Bothrops, Calloselasma, Echis and Oxyuranus), such experiments determined that Ru based compounds, separately and in combination, may or may not inhibit procoagulant activity in a synergistic fashion. To reiterate, these enzymes include SP, MP, kallikrein-like SP, and molecules that closely resemble human coagulation factors V (FV) and X (FX) (see, Aguiar, W. D. S.; et al., PLoS One 2019, 14; Tang, E. L.; et al., J Proteomics 2016, 148, 44-56; Patra, A.; et al., Sci Rep 2017, 7, 17119; Yamada, D.; Morita, T. Thromb Res 1999, 94, 221-226; Chen, Y. L.; Tsai, I. H. Biochemistry 1996, 35, 5264-52671; Koludarov, I.; et al., Toxins (Basel) 2014, 6, 3582-3595; Sanggaard, K. W.; et al., J Proteomics 2015, 117, 1-11; Herrera, M.; et al., J Proteomics 2012, 75, 2128-2140; McCleary, R. J.; et al., J Proteomics 2016, 144, 51-62), which are found in the indicated venoms displayed in Table 1. These venoms were chosen as they have already demonstrated marked vulnerability to inhibition by CORM-2 and or RuCl₃ in previous works (see, Nielsen, V. G.; Frank, N. J Thromb Thrombolysis 2019, 47, 533-539; Nielsen, V. G. Int J Mol Sci 2020, 21, 2970; Nielsen, V. G.; Bazzell, C. M. J Thromb Thrombolysis 2017, 43, 203-208; Nielsen, V. G.; Frank, N.; Matika, R. W. Biometals 2018, 31, 51-59; Nielsen, V. G.; Frank, N. Hum Exp Toxicol 2019, 38, 216-226; Nielsen, V. G.; et al., Toxins (Basel) 2019, 11, E94).

TABLE 1
Properties of procoagulant snake venoms investigated.

| Species | Common Name | Proteome |
|---|---|---|
| Bothrops moojeni [27] | Brazilian Lancehead | SP, MP |
| Calloselasma | Malayan Pit Viper | SP, MP |
| rhodostoma [28] | | |
| Echis leucogaster | White-Bellied Carpet | SP, MP |
| [29-31] | Viper | |
| Heloderma suspectum | Gila Monster | Kallikrein-like SP |
| [32, 33] | | |
| Oxyuranus | Inland Taipan | Factor V-like, |
| microlepidotus [34] | | SP, MP |
| Pseudonaja textilis [35] | Eastern Brown Snake | Factor V, X-like |
| | | SP, MP |

Thus, given the aforementioned molecular complexity, experiments conducted during the course of developing embodiments for the present invention (see, Examples I, II, and IV) sought to determine the effects of CORM-2, CORM-3, carboplatin and RuCl₃ exposure (separately and as a formulation) on a variety of diverse procoagulant snake venoms to provide insight into any interactions of the compounds on venom procoagulant activity. Utilizing venom collected from four diverse genera (Bothrops, Calloselasma, Echis and Oxyuranus), such experiments determined that Ru based compounds, separately and in combination, may or may not inhibit procoagulant activity in a synergistic fashion. Indeed, a purpose of this investigation was to determine if formulations of platinoid compounds could inhibit venom procoagulant activity and if the compounds formulated interacted to enhance inhibition. Using a human plasma coagulation kinetic model to assess venom activity, six diverse venoms were exposed to various combinations and concentrations of CORM-2, CORM-3, RuCl₃ and carboplatin (a platinum containing compound) with changes in venom activity determined with thrombelastography. The combinations of CORM-2 or CORM-3 with RuCl₃ were found to enhance inhibition significantly, but not in all venoms nor to the same extent. In sharp contrast, carboplatin antagonized CORM-2 mediated inhibition of venom activity. These preliminary results support the concept that platinoid compounds apparently inhibit venom enzymatic activity at the same or different molecular site and may antagonize inhibition at the same or different sites.

The demonstration that CORMs affect experimental systems by the release of carbon monoxide, and not via the interaction of the inactivated CORM, has been an accepted paradigm for decades. However, it has recently been documented that a radical intermediate formed during carbon monoxide release from ruthenium (Ru)-based CORM (CORM-2) interacts with histidine and can inactivate bee phospholipase A₂ activity. Using a thrombelastographic based paradigm to assess procoagulant activity in human plasma, additional experiments (see, Example III) were conducted that tested the hypothesis that a Ru-based radical and not carbon monoxide was responsible for CORM-2 mediated inhibition of Athens, Echis, and Pseudonaja species snake venoms. Assessment of the inhibitory effects of ruthenium chloride (RuCl₃) on snake venom activity was also determined. CORM-2 mediated inhibition of the three venoms was found to be independent of carbon monoxide release, as the presence of histidine-rich albumin abrogated CORM-2 inhibition. Exposure to RuCl₃ had little effect on Atheris venom activity, but Echis and Pseudonaja venom had procoagulant activity significantly reduced. It was concluded that a Ru-based radical and ion inhibited procoagulant snake venoms, not carbon monoxide. These data continue to add to a mechanistic understanding of how Ru-based molecules can modulate hemotoxic venoms, and these results can serve as a rationale to focus on perhaps other, complementary compounds containing Ru as antivenom agents in vitro and, ultimately, in vivo.

Additional experiments (see Example VI) were conducted to create a rabbit model of subcutaneous envenomation to assess venom toxicodynamics and efficacy of ruthenium based antivenom administration. New Zealand White rabbits were sedated with midazolam via ear vein and had viscoelastic measurements of whole blood and/or plasmatic coagulation kinetics obtained from ear artery samples. Venoms derived from C. scutulatus scutulatus, Bothrops moojeni, or Calloselasma rhodostoma were injected subcutaneously and changes in coagulation determined over three hours and compared to samples obtained prior to envenomation. Other rabbits had ruthenium based antivenoms injected five minutes after venom injection. Viscoelastic analyses demonstrated diverse toxicodynamic patterns of coagulopathy consistent with the molecular composition of the proteomes of the venoms tested. The antivenoms tested attenuated venom mediated coagulopathy. It was concluded that a novel rabbit model can be used to characterize the onset and severity of envenomation by diverse proteomes and to assess site directed antivenoms.

Accordingly, the present invention relates to compositions and methods for treating, ameliorating, and preventing the toxic effects of venom poisoning. In particular, the invention provides compositions comprising one or more ruthenim based-agents for one or more of inhibiting venom related procoagulant activity, inhibiting venom related phospholipase A₂ (PLA₂), and/or inhibiting venom related thrombus generation, and related methods for treating, ameliorating and preventing the toxic effects of venom poisoning in a subject suffering from or at risk of suffering from venom poisoning.

In certain embodiments, the present invention provides compositions comprising one or more ruthenium (Ru)-based agents capable of (e.g., upon in vitro or in vivo exposure to a biological sample) one or more of inhibiting venom related procoagulant activity, inhibiting venom related phospholipase A₂ (PLA₂), and/or inhibiting venom related thrombus generation. In some embodiments, the composition is a pharmaceutical composition.

In some embodiments, the one or more ruthenium-based agents capable of one or more of inhibiting venom related procoagulant activity, inhibiting venom related phospholipase A₂ (PLA₂), and/or inhibiting venom related thrombus generation is a ruthenium compound. In some embodiments, the ruthenium compound is selected from zerovalent, divalent and trivalent ruthenium compounds. In some embodiments, the ruthenium compounds are selected from ruthenium hexafluoride, Ruthenium(IV) Oxide, Ruthenium(VIII) Oxide, Ruthenium(VIII) Oxide, Ruthenium(III) Nitrate, Ruthenium(III) Phosphate, Ruthenium(IV) Sulfate, Ruthenium(II) Nitrate, Ruthenium(IV) Sulfite, Ruthenium(III) Fluoride, Ruthenium(II) Perchlorate, Ruthenium(VI) Sulfide, Ruthenium(III) Nitride, Ruthenium(III) Iodide, Ruthenium Phosphide, Ruthenium(IV) Metasilicate, Ruthenium(III) Acetate, Ruthenium boride, Strontium ruthenate, Lithium ruthenate, Tetrapropylammonium perruthenate, Diruthenium tetraacetate chloride, Uranium ruthenium silicide, Ruthenium hexafluoride, Ruthenium pentafluoride, Cis-Dichlorobis(bipyridine)ruthenium(II), Dicarbonyltris(triphenylphosphine)ruthenium(0), Ruthenium anti-cancer drugs (e.g., KP1019, NAMI-A, Pentaamine(dinitrogen)ruthenium(II) chloride, RAPTA), Ru360 (e.g., an oxo-bridged dinuclear ruthenium ammine complex with an absorption spectrum maximum at 360 nm), Ruthenium red, Ruthenium(III) acetylacetonate, Ruthenium diamine, (Terpyridine)ruthenium trichloride, Tetrasodium tris(bathophenanthroline disulfonate)ruthenium(II), Tris(bipyridine)ruthenium(II) chloride, triruthenium(0) dodecacarbonyl, dichloro(benzene)ruthenium(II) dimer, dichloro(p-cymene)ruthenium(II) dimer, dichloro(mesitylene)ruthenium(II) dimer, dichloro(hexamethylbenzene)ruthenium(II) dimer, diiodo(p-cymene)ruthenium(II) dimer, dipivalato(p-cymene)ruthenium(II), bis(.pi.-methallyl)(1,5-cyclooctadiene)ruthenium(II), dichloro(1,5-cyclooctadiene)ruthenium(II) polymer, dichloro(norbomadiene)ruthenium(II) polymer, dichlorotris(triphenylphosphine)ruthenium(II), chlorohydridotris(triphenylphosphine)ruthenium(II) toluene adduct, dihydridotetrakis(triphenylphosphine)ruthenium(II), carbonylchlorohydridotris(triphenylphosphine)ruthenium(II), carbonyldihydridotris(triphenylphosphine)ruthenium(II), dichlorotetrakis(dimethylsulfoxide)ruthenium(II), ruthenium(III) chloride, ruthenium(III) chloride hydrate, ruthenium(III) iodide, ruthenium(III) iodide hydrate, hexaammineruthenium(III) trichloride, and ruthenium(III) acetylacetonate.

In some embodiments, the ruthenium compound is a ruthenium halide. Examples of ruthenium halides include, but are not limited to, RuCl₃, RuCl₃·H₂O, RuI₃ and hydrated RuBr₃.

In some embodiments, the ruthenium compound has at least one at least one tertiary phosphine ligand. Examples of ruthenium compounds having at least one tertiary phosphine ligand include, but are not limited to, Ru(CO)₃(PPh₃)₂, RuCl₂(CO)₂(PPh₃)₂, RuCl₂(PPh₃)₄, RuH₂(PPh₃)₄, Ru(CH₂═CH₂)(PPh₃)₃, RuHCl(PPh₃)₃·C₇H₈ complex and RuHCl(PPh₃)₃.

In some embodiments, the one or more ruthenium based-agents comprise a Ru-based radical and ion. In some embodiments, the one or more Ru based-agents comprise a Ru-based radical intermediate formed during carbon monoxide release from any Ru-based carbon-monoxide releasing molecule (e.g., tricarbonyldichlororuthenium(II) dimer (CORM-2) and tricarbonylchloro(glycinato)ruthenium (CORM-3). In some embodiments, Ru-based radical and ion is derived from any Ru-based compound.

In some embodiments, the one or more ruthenium based-agents comprise a combination of agents having varying valences. Such compositions are not limited to a specific combination of agents having varying valences. In some embodiments, the composition comprises a first agent having a valence of two, and a second agent having a valence of three. In some embodiments, the agents are ruthenium based compounds. In some embodiments, the first agent having a valence of two is selected from tricarbonyldichlororuthenium(II) dimer (CORM-2) and tricarbonylchloro(glycinato)ruthenium (CORM-3). In some embodiments, the second agent having a valence of three is selected from RuCl₃ (Ru(III), New Anticancer Metastasis Inhibitor (NAMI-A), and trans-[tetrachlorobis(1H-indazole)ruthenate(III) (KP1019). In some embodiments, the composition comprises a combination of CORM-2 and RuCl₃.

In some embodiments, the amounts of the first agent having a valence of two, and a second agent having a valence of three within the composition is such that upon administration to a subject (e.g., a human subject), the composition is able to treat, ameliorate and/or prevent the toxic effects of venom poisoning, and/or treat, ameliorate and/or prevent the toxic effects of PLA₂ activity.

In some embodiments, the amount of the first agent having a valence of two, and a second agent having a valence of three within the composition is such that upon administration to a subject (e.g., a human subject), the composition is able to prevent one or more of venom mediated catalysis of fibrinogen in the subject, venom related PLA₂ activity, venom mediated degradation of plasma coagulation in the subject, venom mediated coagulopathy in the subject, and venom mediated catalysis and inactivation of fibrinogen.

In some embodiments, the amount of the first agent having a valence of two, and a second agent having a valence of three within the composition is such that upon administration to a subject (e.g., a human subject), the composition is able to inhibit venom related procoagulant activity, inhibit venom related PLA₂ activity, and/or inhibit venom related thrombus generation. In some embodiments, such inhibition of venom related procoagulant activity, venom related PLA₂ activity, and/or venom related thrombus generation results in prevention and/or alleviation of pain and neurological effects related to snake venom activity.

In certain embodiments, the present invention provides methods of treating and/or preventing a condition related to PLA₂ activity in a subject comprising administering to the subject a composition comprising one or more Ru-based agents (e.g., a composition comprising a Ru-based radical intermediate formed during carbon monoxide release from any Ru-based carbon-monoxide releasing molecule) (e.g., a composition comprising a first agent having a valence of two, and a second agent having a valence of three (as described herein) (e.g., CORM-2 and RuCl₃)), wherein the administering results in prevention of PLA₂ activity in the subject.

In some embodiments the PLA₂ activity is venom-related PLA₂ activity.

In some embodiments, the condition related to PLA₂ activity is venom poisoining.

In some embodiments, the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, and/or inhibition of venom related thrombus generation.

In certain embodiments, the present invention provides methods of enhancing coagulation or reducing fibrinolysis in a subject suffering from or at risk of suffering from venom poisoning, comprising administering to the subject a composition comprising one or more Ru-based agents (e.g., a composition comprising a Ru-based radical intermediate formed during carbon monoxide release from any Ru-based carbon-monoxide releasing molecule) (e.g., a composition comprising a first agent having a valence of two, and a second agent having a valence of three (as described herein) (e.g., CORM-2 and RuCl₃)), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ acitvity, and/or inhibition of venom related thrombus generation.

Such methods are not limited to a particular type of venom. In some embodiments, the venom is Crotalus related venom. For example, in some embodiments, the Crotalus related venom is a venom from a Crotalus species selected from C. adamanteus, C. aquilus, C. atrox, C. basilicus, C. cerastes, C. durissus, C. enyo, C. horridus, C. intermedius, C. lannomi, C. lepidus, C. mitchellii, C. molossus, C. oreganus, C. polystictus, C. pricei, C. pusillus, C. ruber, C. scutulatus, C. simus, C. stejnegeri, C. tigris, C. tortugensis, C. totonacus, C. transversus, C. triseriatus, C. viridis, and C. willardi. In some embodiments, the venom is from one of the following: Naja naja (Indian cobra), Bothrops asper (Fur-de-lance), Agkistrodon piscivorus piscivorus, Agkistrodon contortrix contortrix, Agkistrodon contortrix laticinctus, Askistrodon contortix pictigaster, Agkistrodon piscivorus leucostoma, Agkistrodon contortrix mokasen, Northern Pacific rattlesnake, Arizona Black rattlesnake, Prairie rattlesnake, Red Diamond rattlesnake, Timber rattlesnake, Eastern Diamondback rattlesnake, and Southern Pacific rattlesnake.

In certain embodiments, the present invention provides methods of enhancing coagulation or reducing fibrinolysis in a subject suffering from or at risk of suffering from Bothrops venom poisoning, comprising administering to the subject a composition comprising one or more Ru-based agents (e.g., a composition comprising a Ru-based radical intermediate formed during carbon monoxide release from any Ru-based carbon-monoxide releasing molecule) (e.g., either a composition comprising CORM-2 alone or a combination of CORM-2 and RuCl₃ (Ru(III))), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ acitvity, and/or inhibition of venom related thrombus generation.

In certain embodiments, the present invention provides methods of enhancing coagulation or reducing fibrinolysis in a subject suffering from or at risk of suffering from Calloselasma, Echis, or P. textilis venom poisoning, comprising administering to the subject a composition comprising one or more Ru-based agents (e.g., a composition comprising a Ru-based radical intermediate formed during carbon monoxide release from any Ru-based carbon-monoxide releasing molecule) (e.g., either a composition comprising CORM-2 or CORM-3 alone, RuCl₃ alone, or a combination of CORM-2 or CORM-3 and RuCl₃), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ acitvity, and/or inhibition of venom related thrombus generation.

In certain embodiments, the present invention provides methods of enhancing coagulation or reducing fibrinolysis in a subject suffering from or at risk of suffering from Oxyuranus venom poisoning, comprising administering to the subject either a composition comprising one or more Ru-based agents (e.g., a composition comprising a Ru-based radical intermediate formed during carbon monoxide release from any Ru-based carbon-monoxide releasing molecule) (e.g., a composition comprising a combination of CORM-2 and RuCl₃), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ acitvity, and/or inhibition of venom related thrombus generation.

In some embodiments, any of the described compositions (e.g., a composition comprising a Ru-based radical intermediate formed during carbon monoxide release from any Ru-based carbon-monoxide releasing molecule) (e.g., a first agent having a valence of two, and a second agent having a valence of three) (e.g., CORM-2 or CORM-3 alone, RuCl₃ alone, or a combination of CORM-2 or CORM-3 and RuCl₃) are formulated for administration by an aerosol spray, an ointment, a bandage, a surgical dressing, a wound packing, a patch, autoinjector, a swab, a liquid, a paste, a cream, a lotion, a foam, a gel, an emulsion, a powder, or a needle.

In some embodiments, any of the described compositions can be co-administered with a hemostatic agent, a coagulant, an anti-fibrinolytic medication, a blood coagulation factor, fibrin, thrombin, recombinant activated factor VII, prothrombin complex concentrate, FEIBA, or a therapeutic agent selected from the group consisting of an antibiotic, an anesthetic, an analgesic, an antihistamine, an antimicrobial, an antifungal, an antiviral, and an anti-inflammatory agent. In some embodiments, the blood coagulation factor is factor VIII, factor IX, factor XIII, or von Willebrand's factor.

In some embodiments, any of the described compositions can be co-administered with antivenom against the specific type of venom.

In some embodiments, the treated subject is a living mammal (e.g., a living human).

In certain embodiments, the present invention provides kits comprising any of the described compositions (e.g., a composition comprising a Ru-based radical intermediate formed during carbon monoxide release from any Ru-based carbon-monoxide releasing molecule) (e.g., a first agent having a valence of two, and a second agent having a valence of three) (e.g., CORM-2 or CORM-3 alone, RuCl₃ alone, or a combination of CORM-2 or CORM-3 and RuCl₃), an antivenom composition, and instructions for administering the composition to a living mammal. In some embodiments, the kits further comprise one or more of a hemostatic agent, a coagulant, an anti-fibrinolytic medication, a blood coagulation factor, fibrin, thrombin, recombinant activated factor VII, prothrombin complex concentrate, FEIBA, or a therapeutic agent selected from the group consisting of an antibiotic, an anesthetic, an analgesic, an antihistamine, an antimicrobial, an antifungal, an antiviral, and an anti-inflammatory agent.

### BRIEF DESCRIPTION OF DRAWINGS

FIG. 1. Ruthenium based compounds and platinum (Pt)-based utilized in the present investigation.

FIG. 2. Thrombelastographic effects of exposure of Bothrops moojeni venom to CORM-2 and RuCl₃. Data are displayed as mean±SD. TMRTG=minutes, a measure of speed of onset of coagulation; MRTG=dynes/cm²/sec, a measure of the velocity of clot growth; TTG=dynes/cm², a measure of clot strength. V condition—venom in PBS; Ru(II) condition—venom exposed to CORM-2; Ru(III) condition—venom exposed to RuCl₃; Ru(II+III) condition—venom exposed to CORM-2 and RuCl₃ simultaneously. *P<0.05 vs. V; †P<0.05 vs. Ru(II); ‡P<0.05 vs. Ru(III). Significant results of two-way ANOVA indicated by Ru(II)×Ru (III) within the figure.

FIG. 3. Thrombelastographic effects of exposure of Calloselasma rhodostoma venom to CORM-2 and RuCl₃. Data are displayed as mean±SD. TMRTG=minutes, a measure of speed of onset of coagulation; MRTG=dynes/cm²/sec, a measure of the velocity of clot growth; TTG=dynes/cm², a measure of clot strength. V condition—venom in PBS; Ru(II) condition—venom exposed to CORM-2; Ru(III) condition—venom exposed to RuCl₃; Ru(II+III) condition—venom exposed to CORM-2 and RuCl₃ simultaneously. *P<0.05 vs. V; †P<0.05 vs. Ru(II); ‡P<0.05 vs. Ru(III). Significant results of two-way ANOVA indicated by Ru(II)×Ru (III) within the figure.

FIG. 4. Thrombelastographic effects of exposure of Echis leucogaster venom to CORM-2 and RuCl₃. Data are displayed as mean±SD. TMRTG=minutes, a measure of speed of onset of coagulation; MRTG=dynes/cm²/sec, a measure of the velocity of clot growth; TTG=dynes/cm², a measure of clot strength. V condition—venom in PBS; Ru(II) condition—venom exposed to CORM-2; Ru(III) condition—venom exposed to RuCl₃; Ru(II+III) condition—venom exposed to CORM-2 and RuCl₃ simultaneously. *P<0.05 vs. V; †P<0.05 vs. Ru(II); ‡P<0.05 vs. Ru(III). Significant results of two-way ANOVA indicated by Ru(II)×Ru (III) within the figure.

FIG. 5. Thrombelastographic effects of exposure of Oxyuranus microlepidotus venom to CORM-2 and RuCl₃. Data are displayed as mean±SD. TMRTG=minutes, a measure of speed of onset of coagulation; MRTG=dynes/cm²/sec, a measure of the velocity of clot growth; TTG=dynes/cm², a measure of clot strength. V condition—venom in PBS; Ru(II) condition—venom exposed to CORM-2; Ru(III) condition—venom exposed to RuCl₃; Ru(II+III) condition—venom exposed to CORM-2 and RuCl₃ simultaneously. *P<0.05 vs. V; †P<0.05 vs. Ru(II); ‡P<0.05 vs. Ru(III).

FIG. 6. The conditions (X-axis) are: V=venom without additives; Ru(II)=venom exposed to CORM-3; Ru(III)=venom exposed to RuCl₃; and, Ru(II+III)=venom exposed to both compounds. All three coagulation kinetic parameters demonstrated significant interactions between CORM-3 and RuCl₃ as determined with 2-way analysis of variance (ANOVA). Specifically, the small time to maximum thrombin generation value (TMRTG, min, the time to onset of fastest coagulation) caused by venom alone was significantly increased by CORM-3 or RuCl₃ individually, but increased far more by the combination of the two compounds. With regard to the maximum rate of thrombus generation (MRTG, dynes/cm²/sec) the very large value observed in plasma exposed to venom alone was significantly decreased by either ruthenium compound, but far more by the combination of the two compounds. Lastly, while the maximum clot strength (TTG, dynes/cm²) was enhanced by either ruthenium compound individually, the combination restored the clot strength back toward values associated with venom alone, which is similar to clot strength in the absence of venom. Six experiments were performed for each condition, with statistical significance between the conditions determined with one-way ANOVA. *P<0.05 vs. V, †P<0.05 vs. Ru(II), ‡P<0.05 vs. Ru(III).

FIG. 7. Procoagulant activity of A. nitschei venom in plasma after exposure to CORM-2 without or with albumin in isolation. Data is presented as mean±SD. Control=no additives; V=venom; VC=V with 100 μM CORM-2 in PBS; VC+A=V with 100 μM CORM-2 in albumin. * p<0.05 vs. control; †p<0.05 vs. V; ‡p<0.05 vs. VC via one-way analysis of variance (ANOVA) with Holm-Sidak post hoc test.

FIG. 8. Procoagulant activity of E. leucogaster venom in plasma after exposure to CORM-2 without or with albumin in isolation. Data are presented as mean±SD. Control=no additives; V=venom; VC=V with 100 μM CORM-2 in PBS; VC+A=V with 100 μM CORM-2 in albumin. * p<0.05 vs. control; †p<0.05 vs. V; ‡p<0.05 vs. VC via one-way ANOVA with Holm-Sidak post hoc test.

FIG. 9. Procoagulant activity of P. textilis venom in plasma after exposure to CORM-2 without or with albumin in isolation. Data are presented as mean±SD. Control=no additives; V=venom; VC=V with 100 μM CORM-2 in PBS; VC+A=V with 100 μM CORM-2 in albumin. * p<0.05 vs. control; †p<0.05 vs. V; ‡p<0.05 vs. VC via one-way ANOVA with Holm-Sidak post hoc test.

FIG. 10. Interactions of RuCl₃ concentration and fluid within which it is dissolved. Data are mean±SD. 1-W=1 μM RuCl₃ in dH₂O; 1-PBS=1 μM RuCl₃ in PBS; 10-W=10 μM RuCl₃ in dH₂O; 10-PBS=10 μM RuCl₃ in PBS. * p<0.05 vs. 1-W; †p<0.05 vs. 1-PBS; ‡p<0.05 vs. 10-W via two-way ANOVA with Holm-Sidak post hoc test. Two-way ANOVA results for interaction of RuCl₃ concentration and fluid are indicated within each panel.

FIG. 11. Effect of exposure of A. nitschei, E. leucogaster, and P. textilis venom to 100 μM RuCl₃ in PBS on TMRTG values in human plasma. Data are presented as mean±SD. White bars=no RuCl₃ exposure; black bars=100 μM RuCl₃ in PBS exposure. * p<0.05 vs. No RuCl₃ in PBS exposure via two-tailed, unpaired t-test.

FIG. 12. Effect of exposure of A. nitschei, E. leucogaster, and P. textilis venom to 100 μM RuCl₃ in PBS on MRTG values in human plasma. Data are presented as mean±SD. White bars=no RuCl₃ exposure; black bars=100 μM RuCl₃ in PBS exposure. * p<0.05 vs. no RuCl₃ in PBS exposure via two-tailed, unpaired t-test.

FIG. 13. Effect of exposure of A. nitschei, E. leucogaster, and P. textilis venom to 100 μM RuCl₃ in PBS on MRTG values in human plasma. Data are presented as mean±SD. White bars=no RuCl₃ exposure; black bars=100 μM RuCl₃ in PBS exposure. * p<0.05 vs. no RuCl₃ in PBS exposure via two-tailed, unpaired t-test.

FIG. 14. Procoagulant activity of B. moojeni venom (left panels) and C. rhodostoma venom (right panels) in plasma after exposure to CORM-2 (Ru(II)), RuCl₃ (Ru(III)) or both (Ru(II+III)) in isolation. Data is presented as mean±SD. V=venom; Ru(II)=V+CORM-2 in PBS; Ru(III)=V+RuCl₃; Ru(II+III)=V+CORM-2 and RuCl₃. *P<0.05 vs. V; †P<0.05 vs. Ru(II); ‡P<0.05 vs. Ru(III) via one-way analysis of variance (ANOVA) with Holm-Sidak post hoc test. Significant interactions between CORM-2 and RuCl₃ determined with two-way ANOVA are displayed within individual parameter graphics.

FIG. 15. Procoagulant activity of E. leucogaster venom (left panels) and O. microlepidotus venom (right panels) in plasma after exposure to CORM-2 (Ru(II)), RuCl₃ (Ru(III)) or both (Ru(II+III)) in isolation. Data is presented as mean±SD. V=venom; Ru(II)=V+CORM-2 in PBS; Ru(III)=V+RuCl₃; Ru(II+III)=V+CORM-2 and RuCl₃. *P<0.05 vs. V; †P<0.05 vs. Ru(II); ‡P<0.05 vs. Ru(III). Significant interactions between CORM-2 and RuCl₃ determined with two-way ANOVA are displayed within individual parameter graphics.

FIG. 16. Procoagulant activity of B. moojeni venom (left panels) and C. rhodostoma venom (right panels) in plasma after exposure to CORM-3 (Ru(II)), RuCl₃ (Ru(III)) or both (Ru(II+III)) in isolation. Data is presented as mean±SD. V=venom; Ru(II)=V+CORM-3 in PBS; Ru(III)=V+RuCl₃; Ru(II+III)=V+CORM-3 and RuCl₃. *P<0.05 vs. V; †P<0.05 vs. Ru(II); ‡P<0.05 vs. Ru(III). Significant interactions between CORM-3 and RuCl₃ determined with two-way ANOVA are displayed within individual parameter graphics.

FIG. 17. Procoagulant activity of P. textilis venom (left panels) and H. suspectum venom (right panels) in plasma after exposure to CORM-3 (Ru(II)), RuCl₃ (Ru(III)) or both (Ru(II+III)) in isolation. Data is presented as mean±SD. V=venom; Ru(II)=V+CORM-3 in PBS; Ru(III)=V+RuCl₃; Ru(II+III)=V+CORM-3 and RuCl₃. *P<0.05 vs. V; †P<0.05 vs. Ru(II); ‡P<0.05 vs. Ru(III). Significant interactions between CORM-3 and RuCl₃ determined with two-way ANOVA are displayed within individual parameter graphics.

FIG. 18. Procoagulant activity of B. moojeni venom (left panels) and C. rhodostoma venom (right panels) in plasma after exposure to carboplatin (Pt(II)), CORM-2 (Ru(II)), or both (Pt+Ru) in isolation. Data is presented as mean±SD. V=venom; Pt(II)=V+carboplatin in PBS; Ru(II)=V+CORM-2; Pt+Ru=V+carboplatin and CORM-2. *P<0.05 vs. V; †P<0.05 vs. Pt(II); ‡P<0.05 vs. Ru(II) via one-way analysis of variance (ANOVA) with Holm-Sidak post hoc test. Significant interactions between carboplatin and CORM-2 determined with two-way ANOVA are displayed within individual parameter graphics.

FIG. 19. Effects of RuCl3 on the anticoagulant activity of Mojave rattlesnake venom type A in human plasma. Data are displayed as mean±SD. TMRTG=minutes, a measure of speed of onset of coagulation; MRTG=dynes/cm²/sec, a measure of the velocity of clot growth; TTG=dynes/cm², a measure of clot strength. C=control condition—no RuCl₃ or venom addition; 2) R/W=1% addition of 100 μM RuCl₃ dissolved in water; R/P=1% addition of 100 μM RuCl₃ dissolved in PBS; V=1% venom addition (125 ng/ml final concentration) in PBS; and, V+R/P=1% addition of venom exposed to RuCl₃ in PBS. *P<0.05 vs. C; †P<0.05 vs. R/W, ‡P<0.05 vs. R/P, § P<0.05 vs. V.

FIG. 20. Panel A—Rabbit model of envenomation. Blue area indicates site of venom injection, with green indicating the antivenom injection. Panel B—The Mojave rattlesnake, Crotalus scutulatus scutulatus. Panel C—The Brazilian lancehead, Bothrops moojeni. Panel D—The Malayan pit viper, Calloselasma rhodostoma. The photographs of the snakes were kindly provided by the National Natural Toxins Research Center at Texas A&M University-Kingsville, Kingsville, Texas, USA.

FIG. 21. Thrombelastographic parameters displayed in clot growth velocity curves of rabbit whole blood and plasma. Typical classic, corresponding recordings of clot formation are displayed in the right side of the diagram. See the preceding text for definitions of the thrombelastographic variables.

FIG. 22. Comparison of whole blood and plasmatic coagulation after C. scutulatus scutulatus envenomation. Left panel—There was no significant change in whole blood coagulation following evenomation with C. scutulatus scutulatus venom. Right panel—C. scutulatus scutulatus venom injection resulted in a significant decrease in MRTG (dynes/cm²/second) and TTG (dynes/cm²) without affecting TMRTG (minutes). Data presented as mean±SD; 1H, 2H, and 3H are hours after venom injection; *P<0.05 vs. baseline; †P<0.05 vs. 1H.

FIG. 23. Contribution of platelets to clot strength after C. scutulatus scutulatus envenomation. Data presented as mean±SD.

FIG. 24. Effects of injection of CORM-2 into the C. scutulatus scutulatus envenomation site on plasmatic coagulation kinetics. Left panel—10 mg/kg CORM-2 injected (n=1); Right panel—20 mg/kg CORM-2 injected (n=1).

FIG. 25. Effects of B. moojeni venom injection without or with antivenom treatment on whole blood coagulation. V (black bars)=rabbits injected with venom; V+A (white bars)=rabbits administered antivenom after venom injection. TMRTG (minutes); MRTG (dynes/cm²/second; TTG (dynes/cm²). Data presented as mean±SD; 1H, 2H, and 3H are hours after venom injection; *P<0.05 vs. baseline; †P<0.05 vs. 1H; ‡P<0.05 vs. 2H; #P<0.05 vs. V. AV×Time=result of two-way ANOVA to determine interaction of time with antivenom administration.

FIG. 26. Effects of C. rhodostoma venom injection without or with antivenom treatment on whole blood coagulation. V (black bars)=rabbits injected with venom; V+A (white bars)=rabbits administered antivenom after venom injection. TMRTG (minutes); MRTG (dynes/cm²/second; TTG (dynes/cm²). Data presented as mean±SD; 1H, 2H, and 3H are hours after venom injection; *P<0.05 vs. baseline; †P<0.05 vs. 1H; ‡P<0.05 vs. 2H; #P<0.05 vs. V. AV×Time=result of two-way ANOVA to determine interaction of time with antivenom administration.

### DEFINITIONS

The term “venom” is intended to encompass any poisonous substance which is parenterally transmitted, that is subcutaneously or intramuscularly transmitted, by the bite or sting of a venomous animal into a mammal and which contains various toxins such as, but not limited to, hemotoxins, hemagglutinins, neurotoxins, leukotoxins, and endotheliatoxins.

The term “venomous animals” is taken to mean venomous members of the Animal kingdom, as are well known in the art. Non-limiting examples of venomous animals whose bite or sting transmit venom to a mammal victim include reptiles such as snakes. Non-limiting examples of venomous snakes include, but are not limited to, Bothrops spp., Calloselasma spp., Echis spp., and Oxyuranus spp.

The term “subject” or “patient” who is suffering from the bite or sting of a venomous animal is a mammal, preferably humans, and includes household pets and livestock, including but not limited to dogs, cats, sheep, horses, cows, goats, and pigs.

### DETAILED DESCRIPTION OF THE INVENTION

The present invention relates to compositions and methods for treating, ameliorating, and preventing the toxic effects of venom poisoning. In particular, the invention provides compositions comprising one or more ruthenim based-agents for one or more of inhibiting venom related procoagulant activity, inhibiting venom related phospholipase A₂ (PLA₂), and/or inhibiting venom related thrombus generation, and related methods for treating, ameliorating and preventing the toxic effects of venom poisoning in a subject suffering from or at risk of suffering from venom poisoning.

Snake venoms, produced primarily for the procurement of prey or in a defensive role, are complex biological mixtures of upwards of 50 components. Death of prey from a snake bite is due to respiratory or circulatory failure caused by various neurotoxins, cardiotoxins (also called cytotoxins), coagulation factors, and other substances acting alone or synergistically. Snake venoms also contain a number of enzymes which when injected into the prey start tissue digestion. The venoms thus contain substances designed to affect the vital processes such as nerve and muscle function, the action of the heart, circulation of the blood and the permeability of membranes. Most constituents of snake venoms are proteins, but low molecular weight compounds such as peptides, nucleotides and metal ions are also present.

Poisonous (venomous) snakes may be divided into 4 main families, the Colubridae, the Viperidae, the Hydrophidae and the Elapidae. Rattlesnakes which are particular to the American continent are members of a subfamily of venomous snakes from the Viperidae family known as Crotalinae, genera Crotalus or Sistrusus (rattlesnakes), Bothrops, Apkistrodon and Trimerisurus. The two rattlesnake genera may be broken down still further into species and sub species. These snakes are also called the “pit vipers” due to the presence of facial sensory heat pits; however their most prominent feature is the rattle which when present distinguishes them from all other snakes. Each species or subspecies occupies a distinct geographical location in the North or South America. The venom of each species of rattlesnake contains components which may be common to all rattlesnakes, common to only some smaller groups or may be specific to a single species or subspecies.

The compositions and methods of the present invention are not limited to treating, ameliorating and preventing the toxic effects of a particular type of venom. In some embodiments, the venom is any type of venom that inhibits coagulation in a subject. In some embodiments, the venom is any type of venom that causes fibrinolysis in a subject. In some embodiments, the venom is any type of venom that causes catalysis of fibrinogen in a subject. In some embodiments, the venom is any type of venom that causes degradation of plasma coagulation in a subject. In some embodiments, the venom is any type of venom that causes inactivation of fibrinogen in a subject. In some embodiments, the venom is any type of venom that causes one or more of the following in a subject (e.g., a subject suffering from venom poisoning): coagulation inhibition, PLA₂ activitiy inhibition, fibrinolysis, fibrinogen catalysis, plasma coagulation degradation, and fibrinogen inactivation.

In certain embodiments, the present invention provides compositions comprising one or more ruthenium (Ru)-based agents capable of (e.g., upon in vitro or in vivo exposure to a biological sample) one or more of inhibiting venom related procoagulant activity, inhibiting venom related phospholipase A₂ (PLA₂), and/or inhibiting venom related thrombus generation. In some embodiments, the composition is a pharmaceutical composition.

In some embodiments, the one or more ruthenium-based agents capable of one or more of inhibiting venom related procoagulant activity, inhibiting venom related phospholipase A₂ (PLA₂), and/or inhibiting venom related thrombus generation is a ruthenium compound. In some embodiments, the ruthenium compound is selected from zerovalent, divalent and trivalent ruthenium compounds. In some embodiments, the ruthenium compounds are selected from ruthenium hexafluoride, Ruthenium(IV) Oxide, Ruthenium(VIII) Oxide, Ruthenium(VIII) Oxide, Ruthenium(III) Nitrate, Ruthenium(III) Phosphate, Ruthenium(IV) Sulfate, Ruthenium(II) Nitrate, Ruthenium(IV) Sulfite, Ruthenium(III) Fluoride, Ruthenium(II) Perchlorate, Ruthenium(VI) Sulfide, Ruthenium(III) Nitride, Ruthenium(III) Iodide, Ruthenium Phosphide, Ruthenium(IV) Metasilicate, Ruthenium(III) Acetate, Ruthenium boride, Strontium ruthenate, Lithium ruthenate, Tetrapropylammonium perruthenate, Diruthenium tetraacetate chloride, Uranium ruthenium silicide, Ruthenium hexafluoride, Ruthenium pentafluoride, Cis-Dichlorobis(bipyridine)ruthenium(II), Dicarbonyltris(triphenylphosphine)ruthenium(0), Ruthenium anti-cancer drugs (e.g., KP1019, NAMI-A, Pentaamine(dinitrogen)ruthenium(II) chloride, RAPTA), Ru360 (e.g., an oxo-bridged dinuclear ruthenium ammine complex with an absorption spectrum maximum at 360 nm), Ruthenium red, Ruthenium(III) acetylacetonate, Ruthenium diamine, (Terpyridine)ruthenium trichloride, Tetrasodium tris(bathophenanthroline disulfonate)ruthenium(II), Tris(bipyridine)ruthenium(II) chloride, triruthenium(0) dodecacarbonyl, dichloro(benzene)ruthenium(II) dimer, dichloro(p-cymene)ruthenium(II) dimer, dichloro(mesitylene)ruthenium(II) dimer, dichloro(hexamethylbenzene)ruthenium(II) dimer, diiodo(p-cymene)ruthenium(II) dimer, dipivalato(p-cymene)ruthenium(II), bis(.pi.-methallyl)(1,5-cyclooctadiene)ruthenium(II), dichloro(1,5-cyclooctadiene)ruthenium(II) polymer, dichloro(norbomadiene)ruthenium(II) polymer, dichlorotris(triphenylphosphine)ruthenium(II), chlorohydridotris(triphenylphosphine)ruthenium(II) toluene adduct, dihydridotetrakis(triphenylphosphine)ruthenium(II), carbonylchlorohydridotris(triphenylphosphine)ruthenium(II), carbonyldihydridotris(triphenylphosphine)ruthenium(II), dichlorotetrakis(dimethylsulfoxide)ruthenium(II), ruthenium(III) chloride, ruthenium(III) chloride hydrate, ruthenium(III) iodide, ruthenium(III) iodide hydrate, hexaammineruthenium(III) trichloride, and ruthenium(III) acetylacetonate.

In some embodiments, the ruthenium compound is a ruthenium halide. Examples of ruthenium halides include, but are not limited to, RuCl₃, RuCl₃H₂O, RuI₃ and hydrated RuBr₃.

In some embodiments, the ruthenium compound has at least one at least one tertiary phosphine ligand. Examples of ruthenium compounds having at least one tertiary phosphine ligand include, but are not limited to, Ru(CO)₃(PPh₃)₂, RuCl₂(CO)₂(PPh₃)₂, RuCl₂(PPh₃)₄, RuH₂(PPh₃)₄, Ru(CH₂═CH₂)(PPh₃)₃, RuHCl(PPh₃)₃·C₇H₈ complex and RuHCl(PPh₃)₃.

In some embodiments, the one or more ruthenium based-agents comprise a Ru-based radical and ion. In some embodiments, the one or more Ru based-agents comprise a Ru-based radical intermediate formed during carbon monoxide release from any Ru-based carbon-monoxide releasing molecule (e.g., tricarbonyldichlororuthenium(II) dimer (CORM-2) and tricarbonylchloro(glycinato)ruthenium (CORM-3). In some embodiments, Ru-based radical and ion is derived from any Ru-based compound.

In some embodiments, the one or more ruthenium based-agents comprise a combination of agents having varying valences. Such compositions are not limited to a specific combination of agents having varying valences. In some embodiments, the composition comprises a first agent having a valence of two, and a second agent having a valence of three. In some embodiments, the agents are ruthenium based compounds. In some embodiments, the first agent having a valence of two is selected from tricarbonyldichlororuthenium(II) dimer (CORM-2) and tricarbonylchloro(glycinato)ruthenium (CORM-3). In some embodiments, the second agent having a valence of three is selected from RuCl₃ (Ru(III), New Anticancer Metastasis Inhibitor (NAMI-A), and trans-[tetrachlorobis(1H-indazole)ruthenate(III) (KP1019). In some embodiments, the composition comprises a combination of CORM-2 and RuCl₃.

In some embodiments, the compositions comprise a combination of agents having varying valences (e.g., a combination of a first agent with a valence of two and a second agent with a valence of three) (e.g., a combination of ruthenium compound having a valence of two and a ruthemium compound having a valence of three). Such compositions are not limited to a specific combination of agents having varying valences. In some embodiments, the composition comprises a first agent having a valence of two, and a second agent having a valence of three. In some embodiments, the agents are ruthenium based compounds. In some embodiments, the first agent having a valence of two is selected from tricarbonyldichlororuthenium(II) dimer (CORM-2) and tricarbonylchloro(glycinato)ruthenium (CORM-3). In some embodiments, the second agent having a valence of three is selected from RuCl₃ (Ru(III), New Anticancer Metastasis Inhibitor (NAMI-A), and trans-[tetrachlorobis(1H-indazole)ruthenate(III) (KP1019). In some embodiments, the composition comprises a combination of CORM-2 and RuCl₃.

In some embodiments, the venom is selected from Bothrops, Calloselasma, Echis and Oxyuranus.

The present invention is not limited to a particular manner of treating, ameliorating and preventing the toxic effects of venom poisoning. In some embodiments, such methods involve administering to a subject (e.g., a human suffering from or at risk of suffering from a venom poisoning) a composition (e.g., a pharmaceutical composition) comprising a combination of agents having varying valences (e.g., a combination of a first agent with a valence of two and a second agent with a valence of three) (e.g., a combination of ruthenium compound having a valence of two and a ruthemium compound having a valence of three), wherein in vitro or in vivo exposure of the composition to a biological sample results in inhibition of venom related procoagulant activity, inhibition of venom related PLA₂ activity, and/or inhibition of venom related thrombus generation. In some embodiments, the composition comprises a combination of CORM-2 and RuCl₃.

In some embodiments, the the first agent having a valence of two, and a second agent having a valence of three within the composition is such that upon administration to a subject (e.g., a human subject), the composition is able to treat, ameliorate and/or prevent the toxic effects of venom poisoning.

In some embodiments, the amounts of the first agent having a valence of two, and a second agent having a valence of three within the composition is such that upon administration to a subject (e.g., a human subject), the composition is able to prevent one or more of venom mediated catalysis of fibrinogen in the subject, venom mediated degradation of plasma coagulation in the subject, venom mediated coagulopathy in the subject, and venom mediated catalysis and inactivation of fibrinogen.

In some embodiments, the amounts of the first agent having a valence of two, and a second agent having a valence of three within the composition is such that upon administration to a subject (e.g., a human subject), the composition is able to inhibit venom related procoagulant activity, and/or inhibit venom related thrombus generation. In some embodiments, such inhibition of venom related procoagulant activity and/or venom related thrombus generation results in prevention and/or alleviation of pain and neurological effects related to snake venom activity.

Such compositions are not limited to a particular manner of treating, ameliorating and preventing the toxic effects of venom poisoning.

In some embodiments, administration of such a composition to a subject results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen.

In certain embodiments, the present invention provides methods of treating and/or preventing a condition related to PLA₂ activity in a subject comprising administering to the subject a composition comprising one or more Ru-based agents (e.g., a composition comprising a Ru-based radical intermediate formed during carbon monoxide release from any Ru-based carbon-monoxide releasing molecule) (e.g., a composition comprising a first agent having a valence of two, and a second agent having a valence of three (as described herein) (e.g., CORM-2 and RuCl₃)), wherein the administering results in prevention of PLA₂ activity in the subject.

In some embodiments the PLA₂ activity is venom-related PLA₂ activity.

In some embodiments, the condition related to PLA₂ activity is venom poisoining.

In some embodiments, the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, and/or inhibition of venom related thrombus generation.

In certain embodiments, the present invention provides methods of enhancing coagulation or reducing fibrinolysis in a subject suffering from or at risk of suffering from venom poisoning, comprising administering to the subject either a composition comprising a first agent having a valence of two, and a second agent having a valence of three (as described herein) (e.g., CORM-2 and RuCl₃), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ activity, and/or inhibition of venom related thrombus generation.

The methods are not limited to a particular type of venom. In some embodiments, the venom is Crotalus related venom. For example, in some embodiments, the Crotalus related venom is a venom from a Crotalus species selected from C. adamanteus, C. aquilus, C. atrox, C. basilicus, C. cerastes, C. durissus, C. enyo, C. horridus, C. intermedius, C. lannomi, C. lepidus, C. mitchellii, C. molossus, C. oreganus, C. polystictus, C. pricei, C. pusillus, C. ruber, C. scutulatus, C. simus, C. stejnegeri, C. tigris, C. tortugensis, C. totonacus, C. transversus, C. triseriatus, C. viridis, and C. willardi. In some embodiments, the venom is from one of the following: Naja naja (Indian cobra), Bothrops asper (Fur-de-lance), Agkistrodon piscivorus piscivorus, Agkistrodon contortrix contortrix, Agkistrodon contortrix laticinctus, Askistrodon contortix pictigaster, Agkistrodon piscivorus leucostoma, Agkistrodon contortrix mokasen, Northern Pacific rattlesnake, Arizona Black rattlesnake, Prairie rattlesnake, Red Diamond rattlesnake, Timber rattlesnake, Eastern Diamondback rattlesnake, and Southern Pacific rattlesnake.

In certain embodiments, the present invention provides methods of enhancing coagulation or reducing fibrinolysis in a subject suffering from or at risk of suffering from Bothrops venom poisoning, comprising administering to the subject either a composition comprising CORM-2 alone or a combination of CORM-2 and RuCl₃ (Ru(III)), wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ activity, and/or inhibition of venom related thrombus generation.

In certain embodiments, the present invention provides methods of enhancing coagulation or reducing fibrinolysis in a subject suffering from or at risk of suffering from Calloselasma, Echis, or P. textilis venom poisoning, comprising administering to the subject either a composition comprising CORM-2 or CORM-3 alone, RuCl₃ alone, or a combination of CORM-2 or CORM-3 and RuCl₃, wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ activity, and/or inhibition of venom related thrombus generation.

In certain embodiments, the present invention provides methods of enhancing coagulation or reducing fibrinolysis in a subject suffering from or at risk of suffering from Oxyuranus venom poisoning, comprising administering to the subject either a composition comprising a combination of CORM-2 and RuCl₃, wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen. In some embodiments, the administering results in inhibition of venom related procoagulant activity, inhibition of venom related PLA₂ activity, and/or inhibition of venom related thrombus generation.

In some embodiments, any of the described compositions (e.g., a first agent having a valence of two, and a second agent having a valence of three) (e.g., CORM-2 or CORM-3 alone, RuCl₃ alone, or a combination of CORM-2 or CORM-3 and RuCl₃) are formulated for administration by an aerosol spray, an ointment, a bandage, a surgical dressing, a wound packing, a patch, autoinjector, a swab, a liquid, a paste, a cream, a lotion, a foam, a gel, an emulsion, a powder, or a needle.

In some embodiments, any of the described compositions can be co-administered with a hemostatic agent, a coagulant, an anti-fibrinolytic medication, a blood coagulation factor, fibrin, thrombin, recombinant activated factor VII, prothrombin complex concentrate, FEIBA, or a therapeutic agent selected from the group consisting of an antibiotic, an anesthetic, an analgesic, an antihistamine, an antimicrobial, an antifungal, an antiviral, and an anti-inflammatory agent. In some embodiments, the blood coagulation factor is factor VIII, factor IX, factor XIII, or von Willebrand's factor.

In some embodiments, any of the described compositions can be co-administered with antivenom against the specific type of venom.

In some embodiments, the treated subject is a living mammal (e.g., a living human).

The compositions described herein can be prepared in a variety of ways. The compositions can be synthesized using various synthetic methods. At least some of these methods are known in the art of synthetic organic chemistry. The compositions described herein can be prepared from readily available starting materials. Optimum reaction conditions can vary with the particular reactants or solvent used, but such conditions can be determined by one skilled in the art.

Reactions to produce the compositions described herein can be carried out in solvents, which can be selected by one of skill in the art of organic synthesis. Solvents can be substantially nonreactive with the starting materials (reactants), the intermediates, or products under the conditions (e.g., temperature and pressure) at which the reactions are carried out. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

One or more of the compositions described herein or pharmaceutically acceptable salts thereof can be provided in a pharmaceutical composition. The pharmaceutical composition can be formulated in accordance with its use and mode of administration. The compositions include a therapeutically effective amount of the first agent having a valence of two, and a second agent having a valence of three within the composition described herein or derivatives thereof in combination with a pharmaceutically acceptable carrier and, optionally, can further include other agents, including other therapeutic agents. These compositions can be prepared in any manner available in the art and can be administered in a number of ways depending on whether local or systemic treatment is desired, on the area to be treated, the subject to be treated, and other variables. Thus, the disclosed compositions can be administered, for example, orally, parenterally (e.g., intravenously), intraventricularly, intramuscularly, intraperitoneally, transdermally, extracorporeally, or topically. The compositions can be administered locally.

By pharmaceutically acceptable is meant a material that is not biologically or otherwise undesirable, which can be administered to an individual along with the selected composition without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions as described herein or pharmaceutically acceptable salts thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compositions described herein or pharmaceutically acceptable salts thereof include capsules, tablets, pills, powders, and granules. In such solid dosage forms, the compositions described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active portions of the compositions in a certain part of the intestinal tract in a delayed manner. Examples of embedding compositions that can be used are polymeric substances and waxes. The active portions of the compositions can also be in micro-encapsulated form, if appropriate, with one or more of the above-mentioned excipients.

Liquid dosage forms for oral administration of the compositions described herein or pharmaceutically acceptable salts thereof include pharmaceutically acceptable emulsions, solutions, suspensions, syrups, and elixirs. In addition to the active portions of the compositions, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active portions of the compositions, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions as described herein or pharmaceutically acceptable salts thereof for rectal administrations are optionally suppositories, which can be prepared by mixing the active portions of the compositions with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the first agent having a valence of two, and a second agent having a valence of three within the composition described herein or pharmaceutically acceptable salts thereof include ointments, powders, sprays, and inhalants. For example, the agents and pharmaceutically acceptable salts thereof can be formulated as a spray for the nasopharynx, the lung, or skin. The agents described herein or pharmaceutically salts thereof are admixed under sterile conditions with a physiologically acceptable carrier and any preservatives, buffers, or propellants as can be required. Ophthalmic formulations, ointments, powders, and solutions are also contemplated as being within the scope of the compositions.

The term pharmaceutically acceptable salts as used herein refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the active portions of the compositions described herein. The term salts refers to the relatively non-toxic, inorganic and organic acid addition salts of the active portions of the compositions described herein. These salts can be prepared in situ during the isolation and purification of the active portions of the compositions or by separately reacting a purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See Stahl and Wermuth, Pharmaceutical Salts: Properties, Selection, and Use, Wiley-VCH, 2008, which is incorporated herein by reference in its entirety, at least, for compositions taught herein).

As disclosed herein, the agents having varying valences (e.g., a combination of a first agent with a valence of two and a second agent with a valence of three) (e.g., a combination of ruthenium compound having a valence of two and a ruthemium compound having a valence of three) and pharmaceutically acceptable salts thereof described herein are useful in treating, ameliorating and/or preventing the toxic effects of venom poisoning. For example, the first agent having a valence of two, and a second agent having a valence of three and pharmaceutically acceptable salts thereof described herein are useful in preventing one or more of venom mediated catalysis of fibrinogen in the subject, venom mediated degradation of plasma coagulation in the subject, venom mediated coagulopathy in the subject, and venom mediated catalysis and inactivation of fibrinogen. The methods described herein comprise selecting a subject suffering from or at risk for suffering from venom related poisoning and administering to a subject an effective amount of a composition described herein or a pharmaceutically acceptable salt thereof. The compositions can be administered locally or systemically in accordance with the subject's needs.

The methods and compositions as described herein are useful for both prophylactic and therapeutic treatment. For prophylactic use, a therapeutically effective amount of a composition described herein and pharmaceutically acceptable salts thereof are administered to a subject at risk of suffering from venom related poisoning. Prophylactic administration can occur for several hours to days prior to such a potential venom poisoning. Prophylactic administration can be used, for example, in preparation for exposure to a region wherein the likelihood for venom poisoning is increased. Therapeutic treatment involves administering to a subject an effective amount of a composition as described herein or pharmaceutically acceptable salts thereof after venom poisoning has commenced.

Administration of the compositionss described herein or pharmaceutically acceptable salts thereof can be carried out using therapeutically effective amounts of the compositions described herein or pharmaceutically acceptable salts thereof for periods of time effective to control the venom poisoning (e.g., the time necessary to enhance coagulation or to reduce fibrinolysis). For example, the compositions described herein or pharmaceutically acceptable salts thereof can be administered as a single dose (i.e., bolus dosage) or as multiple doses.

In some embodiments, the amount of the agents having varying valences (e.g., a combination of a first agent with a valence of two and a second agent with a valence of three) (e.g., a combination of ruthenium compound having a valence of two and a ruthemium compound having a valence of three) in the composition is such that upon administration to a subject (e.g., a human subject), the composition is able to treat, ameliorate and/or prevent the toxic effects of venom poisoning. In some embodiments, the amount of the first agent having a valence of two, and a second agent having a valence of three in the composition is such that upon administration to a subject (e.g., a human subject), the composition is able to prevent one or more of venom mediated catalysis of fibrinogen in the subject, venom mediated degradation of plasma coagulation in the subject, venom mediated coagulopathy in the subject, and venom mediated catalysis and inactivation of fibrinogen.

The method of treating, ameliorating and/or preventing the toxic effects of venom poisoning in a subject can further comprise administering to the subject an additional agent. Thus, the provided compositions and methods can include one or more additional agents. The one or more additional agents or pharmaceutically acceptable salts thereof can be co-administered. Co-administration, as used herein, includes administration in any order, including simultaneous administration, as well as temporally spaced order of up to several days apart. The methods can also include more than a single administration of the one or more additional agents and/or the compositions described herein or pharmaceutically acceptable salts thereof. The administration of the one or more additional agents and the compositions described herein or pharmaceutically acceptable salts can be by the same or different routes and concurrently or sequentially.

The additional agents can include, for example, therapeutic agents. Therapeutic agents include but are not limited to antibiotics, anesthetics, analgesics, antihistamines, antimicrobials, antifungals, antivirals, steroidal and non-steroidal anti-inflammatory agents, chemotherapeutic agents, antibodies, conventional immunotherapeutic agents, cytokines, chemokines, and/or growth factors. In some embodiments, the additional agent is an antivenom (e.g., antivenom against Crotalus venom) (e.g., Crotalidae Polyvalent Immune Fab Ovine (CroFab) or Crotalinae Equine Immune F(ab)2 Antivenom (Anavip)).

Further, the agents having varying valences (e.g., a combination of a first agent with a valence of two and a second agent with a valence of three) (e.g., a combination of ruthenium compound having a valence of two and a ruthemium compound having a valence of three) described herein or pharmaceutically acceptable salts thereof can be co-administered with additional agents that aid in controlling bleeding. For example, the compositions described herein or pharmaceutically acceptable salts thereof can be co-administered with a hemostatic agent, a coagulant, or an anti-fibrinolytic medication. Examples of anti-fibrinolytic agents useful with the methods described herein include aminocaproic acid and tranexamic acid. Other agents that are useful in controlling bleeding, including blood coagulation factors (e.g., factor VIII, factor IX, factor XIII, von Willebrand's factor), fibrin, thrombin, recombinant activated factor VII, prothrombin complex concentrate, and FEIBA (Baxter, Vienna, Austria), can also be co-administered with the compositions described herein or pharmaceutically acceptable salts thereof.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compositions or agents is given first followed by the second). Thus, the term combination is used to refer to either concomitant, simultaneous, or sequential administration of two or more agents.

The agents having varying valences (e.g., a combination of a first agent with a valence of two and a second agent with a valence of three) (e.g., a combination of ruthenium compound having a valence of two and a ruthemium compound having a valence of three) described herein or pharmaceutically acceptable salts thereof, with or without additional agents, can be administered at or near the site of venom poisoning. The agents having varying valences (e.g., a combination of a first agent with a valence of two and a second agent with a valence of three) (e.g., a combination of ruthenium compound having a valence of two and a ruthemium compound having a valence of three) can also be administered, for example, topically, locally, intraveneously, or intramuscularly. Further, the compositions can be formulated for administration, for example, by aerosol sprays, ointments, sutures, bandages, patches, autoinjectors (e.g., similar to epipen autoinjector technology), surgical dressings, wound packings, gauze, swabs, liquids, pastes, creams, lotions, foams, gels, emulsions, or powders. Thus, provided herein are aerosol sprays, ointments, sutures, bandages, patches, autoinjectors, surgical dressings, wound packings, gauze, swabs, liquids, pastes, creams, lotions, foams, gels, emulsions, powders, needles, probes, dental instruments, dental floss, and mouth wash comprising a first agent having a valence of two, and a second agent having a valence of three within the composition.

In certain embodiments, the present invention provides kits comprising any of the described compositions, an antivenom composition, and instructions for administering the composition to a living mammal. In some embodiments, the kits further comprise one or more of a hemostatic agent, a coagulant, an anti-fibrinolytic medication, a blood coagulation factor, fibrin, thrombin, recombinant activated factor VII, prothrombin complex concentrate, FEIBA, or a therapeutic agent selected from the group consisting of an antibiotic, an anesthetic, an analgesic, an antihistamine, an antimicrobial, an antifungal, an antiviral, and an anti-inflammatory agent.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the compositions, and methods of the present invention. Other suitable modifications and adaptations of the variety of conditions and parameters normally encountered in clinical therapy and which are obvious to those skilled in the art are within the spirit and scope of the invention. Use of pronouns such as, “we”, “our,” and “I” refer to the inventive entity.

### Example I

Chemicals and human plasma. Calcium-free phosphate buffered saline (PBS), ruthenium chloride and tricarbonyldichlororuthenium(II) dimer (CORM-2) were obtained from Millipore Sigma (Saint Louis, MO, USA). Venoms dissolved in PBS (50 mg/ml) were obtained from archived, never thawed aliquots maintained at −80° C. in the laboratory that were used in previous investigations (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94). Specifically, Bothrops moojeni (the Brazilian lancehead of South America, (Nielsen, V. G.; Frank, N.; Afshar, S. Toxins (Basel) 2019, 11, E94)) and Calloselasma rhodostoma (the Malayan pitviper of southeast Asia and source of the defibrinating agent ancrod, (Nielsen, V. G.; et al., J Thromb Thrombolysis 2017, 43, 203-208)) venom was obtained originally from the National Natural Toxins Research Center at Texas A&M University (Kingsville, TX, USA). Additionally, Echis leucogaster (white-bellied carpet viper of Africa, (Nielsen V G, Frank N (2019) Hum Exp Toxicol 38, 216-226)) and Oxyuranus microlepidotus (inland taipan of Australia, (Nielsen, V. G.; et al., Biometals 2018, 31, 51-59)) venom was originally purchased from Mtoxins (Oshkosh, WI, USA). Calcium chloride (200 mM) was obtained from Haemonetics Inc., Braintree, MA, USA. Pooled normal human plasma (George King Bio-Medical, Overland Park, KS, USA) that was sodium citrate anticoagulated and maintained at −80° C. was used.

Thrombelastographic analyses. The volumes of subsequently described plasmatic and other additives summed to a final volume of 360 μl. Samples were composed of 320 μl of plasma; 16.4 μl of PBS, 20 μl of 200 mM CaCl₂), and 3.6 μl of PBS or venom mixture, which were pipetted into a disposable cup in a Thrombelastograph® hemostasis system (Model 5000, Haemonetics Inc., Braintree, MA, USA) at 37° C., and then rapidly mixed by moving the cup up against and then away from the plastic pin five times. The following viscoelastic parameters described previously (see, Nielsen V G (2019) J Thromb Thrombolysis 47, 73-79; Gessner G, et al., (2017) Eur J Pharmacol 815, 33-41; Nielsen V G, Wagner M T, Frank N (2020) Int J Mol Sci 21, 2082; Lazić D, Arsenijević A, Puchta R, Bugarči\acute{d} Ž D, Rilak A (2016) Dalton Trans 45, 4633; Hanif M, et al., (2017) ChemPlusChem 82, 841-847) were measured: time to maximum rate of thrombus generation (TMRTG): this is the time interval (minutes) observed prior to maximum speed of clot growth; maximum rate of thrombus generation (MRTG): this is the maximum velocity of clot growth observed (dynes/cm²/second); and total thrombus generation (TTG, dynes/cm²), the final viscoelastic resistance observed after clot formation. Data were collected until a stable maximum amplitude was observed with minimal change for 3 minutes as determined by the software.

Exposures of venoms to RuCl₃ and CORM-2. The aforementioned venoms were exposed to CORM-2 concentrations (or fractions thereof) demonstrated to inhibit procoagulant activity and placed into plasma at the final venom concentrations previously used in this plasma based, thrombelastographic system (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94). Venoms were also exposed to RuCl₃ at 0, 50 or 100 μM concentrations in isolation or in conjunction with CORM-2. The specific exposures for each venom are as follows.

B. moojeni. This venom was exposed to 0 or 1 mM CORM-2 in the presence of 0 or 100 μM RuCl₃ in PBS for at least 5 minutes at room temperature prior to placement into plasma followed immediately with commencement of thrombelastographic analysis. The final concentration of this venom in plasma was 2 μg/ml.

C. rhodostoma. This venom was exposed to 0 or 50 μM CORM-2 in the presence of 0 or 50 μM RuCl₃ in PBS for at least 5 minutes at room temperature prior to placement into plasma followed immediately with commencement of thrombelastographic analysis. The final concentration of this venom in plasma was 5 μg/ml.

E. leucogaster. This venom was exposed to 0 or 100 μM CORM-2 in the presence of 0 or 100 μM RuCl₃ in PBS for at least 5 minutes at room temperature prior to placement into plasma followed immediately with commencement of thrombelastographic analysis. The final concentration of this venom in plasma was 1 μg/ml.

O. microlepidotus. This venom was exposed to 0 or 100 μM CORM-2 in the presence of 0 or 100 μM RuCl₃ in PBS for at least 5 minutes at room temperature prior to placement into plasma followed immediately with commencement of thrombelastographic analysis. The final concentration of this venom in plasma was 1 μg/ml.

Statistical analyses. Data are presented as mean±SD. Graphics were generated with a commercially available program (Origen2020b, OrigenLab Corporation, Northampton, MA, USA). Experimental conditions were composed of n=6 replicates per condition as this provides a statistical power >0.8 with P<0.05 utilizing these techniques (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94). A statistical program was used for one-way analyses of variance (ANOVA) comparisons between conditions, followed by Holm-Sidak post hoc analysis. Additional analysis with two-way ANOVA was performed to detect significant interactions between CORM-2 and RuCl₃ regarding changes in venom procoagulant activity. All analyses were performed with commercial software (SigmaPlot 14, Systat Software, Inc., San Jose, CA, USA). P<0.05 was considered significant.

Given the aforementioned, the experimental conditions utilized were: 1) V condition—venom in PBS; 2) Ru(II) condition—venom exposed to CORM-2; 3) Ru(III) condition—venom exposed to RuCl₃; 4) Ru(II+III) condition—venom exposed to CORM-2 and RuCl₃ simultaneously. After the 5 minute period at room temperature, 3.6 μl of one of these solutions was added to the plasma sample in the plastic thrombelastograph cup.

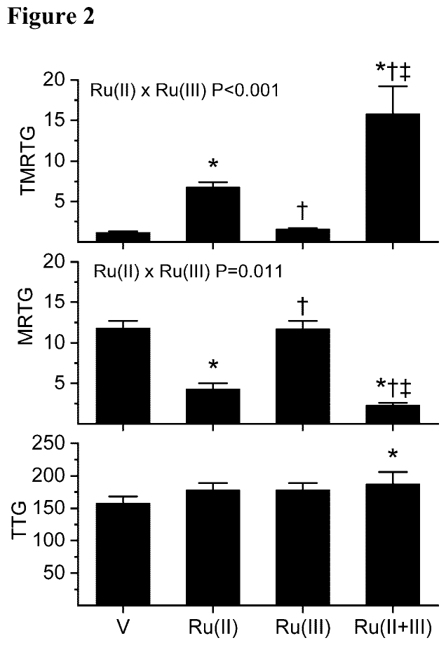

Plasma not exposed to any venom had the following thrombelastographic parameter values: TMRTG=14.0±2.2 minutes, MRTG=2.8±0.8 dynes/cm²/second, TTG=191±13 dynes/cm². This data is provided to simply demonstrate the degree of procoagulant activity each venom tested exerted and was not used in statistical analyses. The data obtained from the aforementioned venom experiments are displayed in FIGS. 2-5. For clarity, each series of venom experiments will be individually presented.

B. moojeni. As seen in FIG. 2, RuCl₃ had no significant effect on the procoagulant activity of this venom; in contrast, venom exposed to CORM-2 had significant loss of activity as evidenced by prolonged TMRTG and decreased MRTG values compared to venom without any chemical exposures. Then, remarkably, the formulation of CORM-2 and RuCl₃ exerted an even greater inhibition of venom procoagulant activity compared to the three other conditions evidenced by the greatest increase in TMRTG, decrease in MRTG, and increased TTG (compared to venom without exposure to additives). Lastly, there was a significant interaction between CORM-2 and RuCl₃ mediated inhibition on venom activity as assessed by changes in TMRTG and MRTG values.

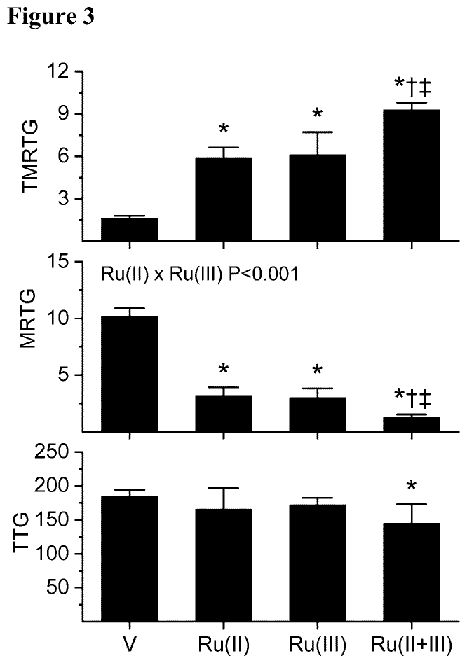

C. rhodostoma. The results of experiments with this venom are displayed in FIG. 3. Exposure of venom to CORM-2 significantly decreased procoagulant activity demonstrated by increased TMRTG and decreased MRTG values compared to CORM-2 naïve venom. The same pattern of inhibition of venom mediated procoagulant activity was observed following exposure of the venom to RuCl₃. Then, exposure of venom to the formulation of CORM-2 and RuCl₃ resulted in an even greater and significant prolongation of TMRTG and decrease of MRTG values compared to the other three conditions. Interestingly, venom exposed to the formulation produced TTG values significantly smaller than the other three conditions. Lastly, there was a significant interaction between CORM-2 and RuCl₃ mediated inhibition on venom activity as determined by changes in MRTG values.

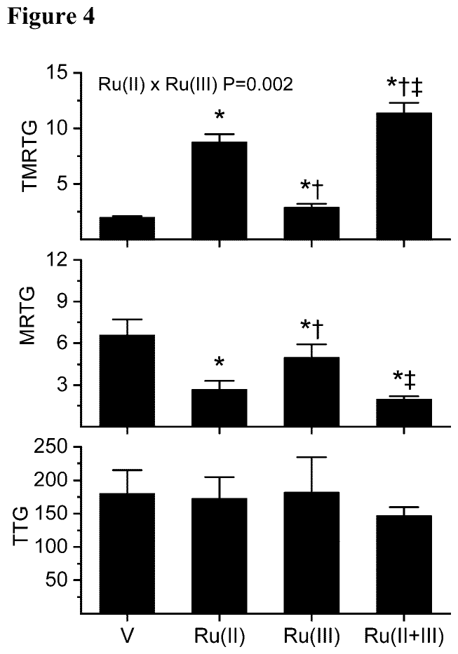

E. leucogaster. The experimental results involving this venom are found in FIG. 4. Exposure of venom to CORM-2 significantly decreased procoagulant activity demonstrated by increased TMRTG and decreased MRTG values compared to CORM-2 naïve venom. The same pattern of inhibition of venom mediated procoagulant activity was observed following exposure of the venom to RuCl₃. Then, exposure of venom to the formulation of CORM-2 and RuCl₃ resulted in an even greater and significant prolongation of TMRTG values compared to the other three conditions. However, the decrease of MRTG values mediated by exposure to the formulation was only significantly different from venom without any chemical exposures or venom exposed to RuCl₃—there was no significant difference from CORM-2 exposed venom. There were no significant changes in TTG across the four conditions. Lastly, there was a significant interaction between CORM-2 and RuCl₃ mediated inhibition on venom activity as determined by changes in TMRTG values.

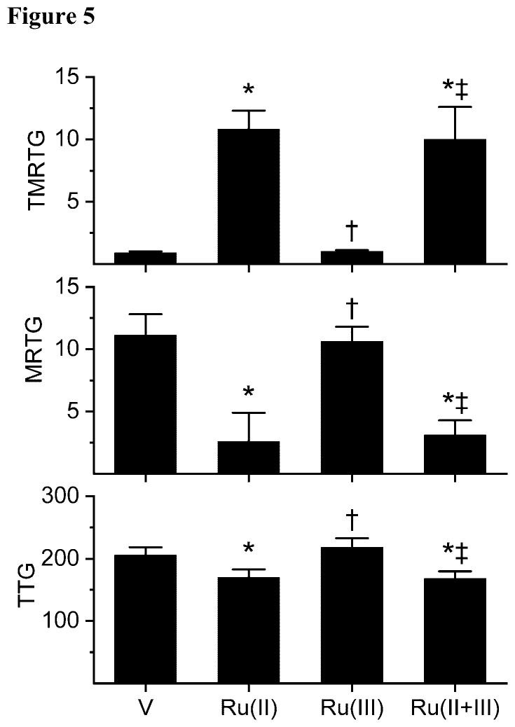

O. microlepidotus. The data obtained from these experiments are found in FIG. 5. As with the other three venoms and previously published results [2], venom exposed to CORM-2 demonstrated a significant decrease in procoagulant activity demonstrated as prolonged TMRTG and decreased MRTG values compared to CORM-2 naïve venom. Then, as with B. moojeni venom, RuCl₃ exposure did not significantly affect the procoagulant activity of this venom. Of interest, and in sharp contrast to the response of the other three venoms, this venom demonstrated decreased procoagulant activity in response to exposure to the formulation of CORM-2 and RuCl₃ that was no different from the inhibition observed following exposure to CORM-2 alone. In sum, there was no discernable effect on the procoagulant activity mediated by RuCl₃ without or with CORM-2 present, and no interaction between CORM-2 and RuCl₃ to either diminish or enhance the procoagulant activity of this venom.

### Example II

This example describes the effects of a formulation of ruthenium chloride and CORM-3 on the procoagulant activity of Pseudonaja textilis (Australian brown snake) venom.

The following experiments used the same methodology as described in Example I. Instead of utilizing CORM-2, these experiments used a compound containing one ruthenium (Ru), CORM-3 (Tricarbonylchloro(glycinato)ruthenium), which contains a Ru (II), just as CORM-2 contains two such Ru (II). The structure of CORM-3 is as follows:

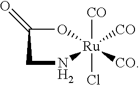

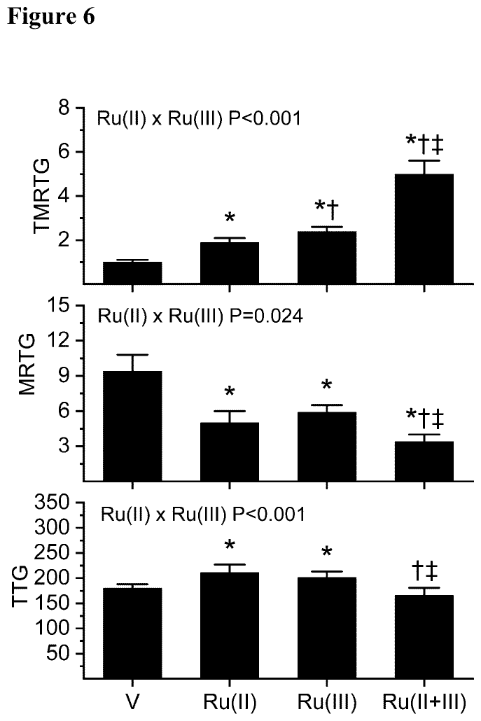

P. textilis venom (10 μg/ml) was exposed to vehicle (dH₂O), RuCl₃ (100 μM), CORM-3 (100 μM), or both RuCl₃ and CORM-3 in phosphate buffered saline at pH=7.4 for five minutes at room temperature. A sample of each of these venom mixtures was then placed into human plasma (0.1 μg/ml final concentration of venom), with coagulation determined by thrombelastography until final clot strength stabilized. The results are shown in FIG. 6.

As shown in FIG. 6, the conditions (X-axis) are: V=venom without additives; Ru(II)=venom exposed to CORM-3; Ru(III)=venom exposed to RuCl₃; and, Ru(II+III)=venom exposed to both compounds. All three coagulation kinetic parameters demonstrated significant interactions between CORM-3 and RuCl₃ as determined with 2-way analysis of variance (ANOVA). Specifically, the small time to maximum thrombin generation value (TMRTG, min, the time to onset of fastest coagulation) caused by venom alone was significantly increased by CORM-3 or RuCl₃ individually, but increased far more by the combination of the two compounds. With regard to the maximum rate of thrombus generation (MRTG, dynes/cm²/sec) the very large value observed in plasma exposed to venom alone was significantly decreased by either ruthenium compound, but far more by the combination of the two compounds. Lastly, while the maximum clot strength (TTG, dynes/cm²) was enhanced by either ruthenium compound individually, the combination restored the clot strength back toward values associated with venom alone, which is similar to clot strength in the absence of venom. Six experiments were performed for each condition, with statistical significance between the conditions determined with one-way ANOVA. *P<0.05 vs. V, †P<0.05 vs. Ru(II), ‡P<0.05 vs. Ru(III).

The finding that the formulation of these two compounds is more effective than either one alone is unexpected, just as such interaction observed between CORM-2 and RuCl₃ was unexpected.

### Example III

This example describes experiments conducted indicting that ruthenium, not carbon monoxide, inhibits the procoagulant activity of Atheris, Echis, and Pseudonaja venoms.

The use of carbon monoxide releasing molecules (CORMs) to deliver carbon monoxide (CO) in a site-directed fashion to presumably alter heme-modulated systems has been part of experimental designs for decades, with hundreds of manuscripts incorporating this methodology. The key element of the paradigm that implicates CO as the mechanism behind the effects of CORMs is the determination that the inactivated releasing molecule (iRM), the portion of the CORM that remains after CO release, has no effect or a different effect on the system tested with the CORM compared to the anticipated CO effect. This laboratory has used this CORM-based paradigm for the past few years to demonstrate that CO inhibited the various procoagulant and anticoagulant activities of hemotoxic venoms and enzymes collected from dozens of snake and lizard species (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41). The particular CORM used was CORM-2 (tricarbonyldichlororuthenium (II) dimer) (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41). The presumed mechanism was that CO must be interacting with a cryptic heme group attached to the various venoms and enzymes or in some other way interacting with these diverse enzymes and venoms (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41). Thus, this potentially heme-based, CO-modulated paradigm of snake venom activity seemed plausible with the aforementioned paradigm of CORM-CO release-inert iRM interactions with target molecules.

However, cracks in the edifice of this paradigm began to appear in the year 2017 with the publication of a work that demonstrated CO-independent inhibition of K⁺ channels with a putative Ru-based radical formed from CORM-2 during CO release and likely prior to formation of its iRM (see, Gessner, G.; et al., Eur. J. Pharmacol. 2017, 815, 33-41). To substantiate this claim, the authors demonstrated that free histidine or albumin, which is resplendent with histidine residues, quenched the inhibition of potassium channels by CORM-2. Furthermore, using mass spectroscopy, the authors demonstrated histidine-Ru-based radical formation following exposure of free histidine with CORM-2. Lastly, using other various CORMs with other metal centers, the authors demonstrated no CO effects on the channel assessed. This laboratory became aware of this work recently, and using a similar approach, demonstrated an identical outcome wherein the anticoagulant activity of the purified phospholipase A₂ (PLA₂) of Apis mellifera venom was inhibited by CORM-2 in a CO-independent, albumin-inhibitable fashion (see, Nielsen, V. G. J. Thromb. Thrombolysis 2020, 49, 100-107). Furthermore, we recently demonstrated that the anticoagulant metalloproteinases of mamba venoms are inhibited by CORM-2 in a CO-independent, albumin-inhibitable manner (see, Nielsen, V. G.; et al., Int. J. Mol. Sci. 2020, 21, 2082). Taken as a whole, it was entirely possible that Ru-based interactions with venom proteins could be responsible for the inhibition noted in our previous works (see, Nielsen, V. G. J. Thromb. Thrombolysis 2019, 47, 73-79); and critically, if Ru-based modifications were the underpinning of such inhibition rather than the interaction of CO with a heme group, then Ru-based CORMs could well serve as permeant antivenom agents. The importance of these line of investigation involving ion channels (see, Gessner, G.; Eur. J. Pharmacol. 2017, 815, 33-41), phospholipase A₂ (see, Nielsen, V. G. J. Thromb. Thrombolysis 2020, 49, 100-107), and metalloproteinases (see, Nielsen, V. G.; et al., Int. J. Mol. Sci. 2020, 21, 2082) is that they lay the foundation to seriously reconsider the paradigm that Ru-based CORMs affect systems as simple as enzymes to as complex as whole animal models of disease in CO-independent ways—potentially affecting the interpretation of data contained in several hundred manuscripts.

While it is unreasonable to reassess all previous venoms inhibited by CORM-2 to determine if a Ru-based radical rather than CO was mediating the inhibition (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41), assessing a few representative venoms would be of benefit. To this end, three procoagulant venoms derived from diverse species from Africa and Australia were selected that have already been characterized as inhibited by CORM-2 but not by its iRM by this laboratory (see, Nielsen, V. G.; Frank, N. Biometals 2018, 31, 951-959; Nielsen, V. G.; Frank, N. Hum Exp. Toxicol. 2019, 38, 216-226). The species chosen are displayed in Table 2, and the venom proteomes of these particular and snakes within the same genus are similar in terms of presence of snake venom serine proteases (SVSP), snake venom metalloproteinases (SVMP), and PLA₂ (see, Wang, H.; Protein J. 2018, 37, 353-360; Patra, A.; et al., Sci. Rep. 2017, 7, 17119; Yamada, D.; Morita, T. Thromb Res. 1999, 94, 221-226; Chen, Y. L.; Tsai, I. H. Biochemistry 1996, 35, 5264-52671; Viala, V. L.; et al., Toxicon 2015, 107 Pt B, 252-265). Fortuitously, archived aliquots of these three venoms that were never thawed or used in the original studies (see, Nielsen, V. G.; Frank, N. Biometals 2018, 31, 951-959; Nielsen, V. G.; Frank, N. Hum Exp. Toxicol. 2019, 38, 216-226) were maintained at −80° C. and were available for the present investigation to test the hypothesis that inhibition by ruthenium molecular species and not carbon monoxide may be the mechanism by which these procoagulant venoms were inhibited by CORM-2.

TABLE 2
Properties of procoagulant snake venoms investigated.

| | | | CORM-2/ |
|---|---|---|---|
| | | | iRM |
| Species | Common Name | Proteome | Inhibition |
| Atheris nitschei | Great Lakes | SVSP, SVMP, | Yes/No |
| | Bush Viper | PLA₂ | |
| Echis leucogaster | White-Bellied | SVSP, SVMP, | Yes/No |
| | Carpet Viper | PLA₂ | |
| Pseudonaja textilis | Eastern | SVSP, SVMP, | Yes/No |
| | Brown Snake | PLA₂ | |

Considering the aforementioned, the following experiments recited within Example III had the following goals. First, determination of inhibition of the procoagulant activities of these venoms by their exposure in isolation to CORM-2 in the absence or presence of albumin was to be performed as previously described with bee venom PLA₂ (see, Nielsen, V. G. J. Thromb. Thrombolysis 2020, 49, 100-107) and mamba venom (see, Nielsen, V. G.; Wagner, M. T.; Frank, N. Int. J. Mol. Sci. 2020, 21, 2082). Second, to further assess if Ru-based molecules may affect venom procoagulant activity, the three venoms were exposed to equimolar concentrations of ruthenium chloride (RuCl₃) which contains a Ru⁺³ state compared to the Ru⁺² state of CORM-2. Compounds incorporating Ru⁺³ more complex than RuCl₃ have been demonstrated to covalently bond to histidine residues in several proteins (see, Messori, L.; Eur. J. Biochem. 2000, 267, 1206-1213; Messori, L.; et al., Met. Based Drugs 2000, 7, 335-342), thus offering the possibility that RuCl₃ could interact with histidine-bearing venom enzymes.

Assessment of the CO-Independent, Ru-Dependent Inhibition of CORM-2 on Procoagulant Activity of A. nitschei, E. leucogaster, and P. textilis Venoms Assessed with Thrombelastography

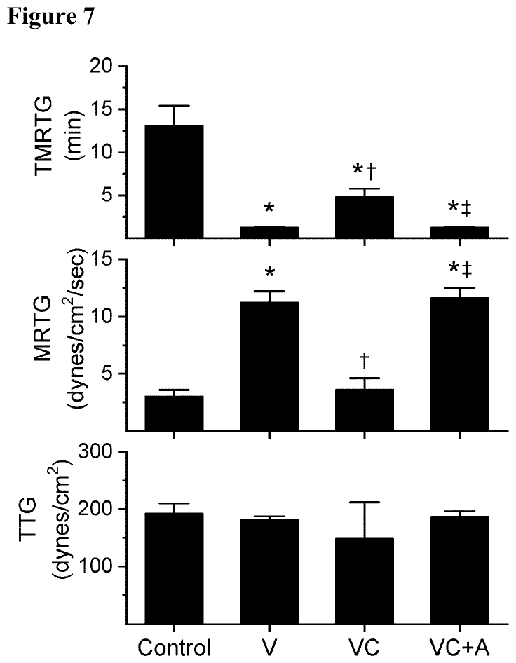

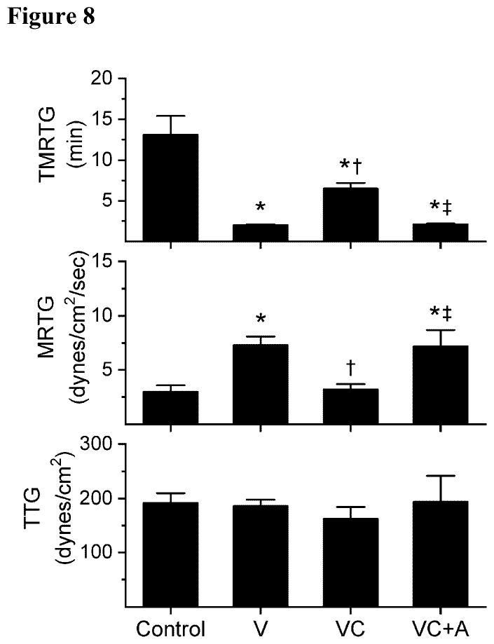

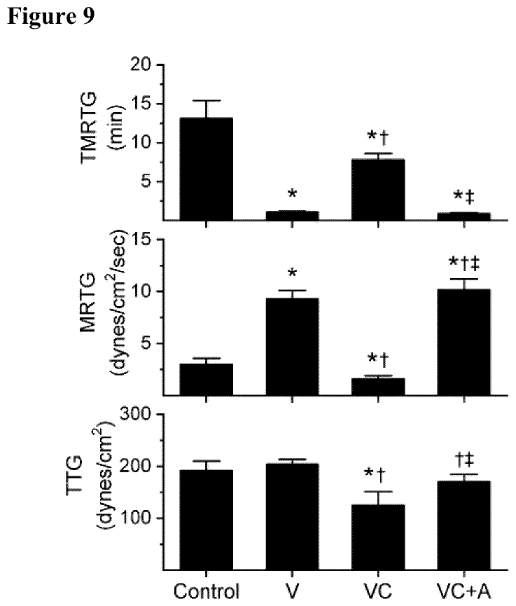

The subsequent results were obtained using concentrations of the aforementioned venoms previously published (see, Nielsen, V. G.; Frank, N. Biometals 2018, 31, 951-959; Nielsen, V. G.; Frank, N. Hum Exp. Toxicol. 2019, 38, 216-226) specifically, A. nitschei and E. leucogaster venoms had a final concentration of 1 μg/mL in the plasma mixtures whereas P. textilis venom was at a final concentration of 100 ng/mL. Venom concentrations were originally chosen based on a performance basis wherein the activation of coagulation by the venom statistically exceeded the activation observed by contact activation with thrombelastographic cup and pin contact with plasma as previously described (see, Nielsen, V. G.; Frank, N. Biometals 2018, 31, 951-959; Nielsen, V. G.; Frank, N. Hum Exp. Toxicol. 2019, 38, 216-226). All venom solutions without or with chemical additions in isolation were added as a 1% addition to the plasma mix used in our thrombelastographic system (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41). This dilution is critical, as it reduces the concentration of CORM-2 to 1 μM, a concentration at which this compound does not affect coagulation kinetics (see, Nielsen, V. G.; et al., Toxins 2019, 11, 94). The thrombelastographic model describes coagulation kinetics with the following three variables: time to maximum thrombus generation (TMRTG, minutes—a measure of time to onset of coagulation), maximum rate of thrombus generation (MRTG, dynes/cm²/s—a measure of the velocity of clot growth) and total thrombus generation (TTG, dynes/cm²—a measure of clot strength). The results of exposing the three venoms to CORM-2 in the absence or presence of 5% human albumin (n=6 for all conditions) are depicted in FIGS. 7, 8 and 9.

All three venoms behaved kinetically as procoagulants as previously noted (see, Nielsen, V. G.; Frank, N. Biometals 2018, 31, 951-959; Nielsen, V. G.; Frank, N. Hum Exp. Toxicol. 2019, 38, 216-226), significantly decreasing TMRTG and increasing MRTG values. Similarly, exposure of the venoms to CORM-2 in PBS significantly attenuated procoagulant activity (see, Nielsen, V. G.; Frank, N. Biometals 2018, 31, 951-959; Nielsen, V. G.; Frank, N. Hum Exp. Toxicol. 2019, 38, 216-226). However, when the venoms were exposed to CORM-2 dissolved in 5% human albumin, procoagulant activity was uninhibited. These results strongly support a CO-independent, Ru-dependent mechanism of inhibition of venom procoagulant activity by CORM-2. Results concerning the effects of RuCl₃ on plasmatic coagulation and venom procoagulant activity are subsequently presented.

### Assessment of the Effects of RuCl₃ on Human Plasmatic Coagulation—Roles of Concentration and Vehicle

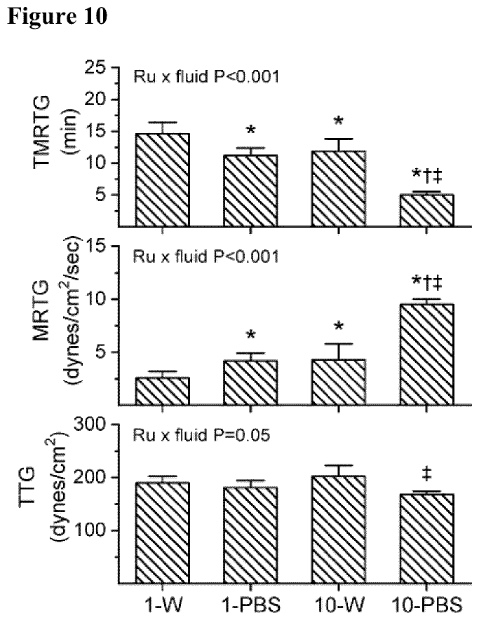

Prior to experimentation with venom, preliminary investigation demonstrated that the expected residual 1 μM concentration of RuCl₃ dissolved in PBS increased MRTG values compared to control conditions. It has already been demonstrated that up to 10 μM CORM-2 in PBS has no effect on plasmatic coagulation kinetics (see, Nielsen, V. G.; et al., Toxins 2019, 11, 94), so a more formal determination of why RuCl₃ displayed procoagulant properties was indicated. Given that the only compounds present with anions different from RuCl₃ potentially available to displace Cl in the PBS used was KH₂PO₄ (1.5 mM) and Na₂HPO₄ (8.1 mM), a comparison of the effects of 1 and 10 μM RuCl₃ (final concentration) dissolved in dH₂O or PBS was performed in plasma as a 1% addition (v/v) with coagulation assessed via thrombelastography. The results of these experiments are displayed in FIG. 10 (n=6 per condition). As can be readily discerned, there appears to be a Ru-dependent, vehicle-independent significant decrease in TMRTG and increase in MRTG values when considering the two-way analysis of variance (ANOVA) results and post hoc comparison of the two concentrations of RuCl₃ dissolved in dH₂O. Similarly, increased RuCl₃ concentrations significantly decrease TMRTG and increase MRTG values when PBS is the vehicle. However, and critically, the coagulation kinetic differences caused by RuCl₃ are significantly enhanced by the fluid it is dissolved in as indicated by the two-way ANOVA significance values in each panel of FIG. 10. Of interest, while TMRTG and MRTG values change in a manner indicative of procoagulation, TTG is decreased by interactions of RuCl₃ and fluid. This thrombelastographic pattern is indicative of enhanced thrombin-fibrinogen interactions without enhanced activation of factor XIII (FXIII) (see, Nielsen, V. G.; et al., Acta Anaesthesiol. Scand. 2005, 49, 222-231; Nielsen, V. G.; et al., Blood Coagul. Fibrinolysis 2007, 18, 145-150).

As all of the venoms investigated over the past few years have been suspended in PBS for the purposes of preserving enzymatic function within a physiological pH, storage, and experimentation (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41; Nielsen, V. G. et al., J. Thromb. Thrombolysis 2020, 49, 100-107; Nielsen, V. G.; et al., Int. J. Mol. Sci. 2020, 21, 2082), it seemed prudent to continue with the use of PBS in the subsequently described experimental series assessing the effects of RuCl₃ on the three venoms of the present work. The caveat that should be kept in mind was that small enhancements of MRTG in human plasma could be secondary to a RuCl₃/PBS interaction when determining if RuCl₃ inhibited venom procoagulant activity.

Assessment of RuCl₃-Dependent Modulation of CORM-2 on the Procoagulant Activity of A. nitschei, E. Leucogaster, and P. textilis Venoms Assessed with Thrombelastography

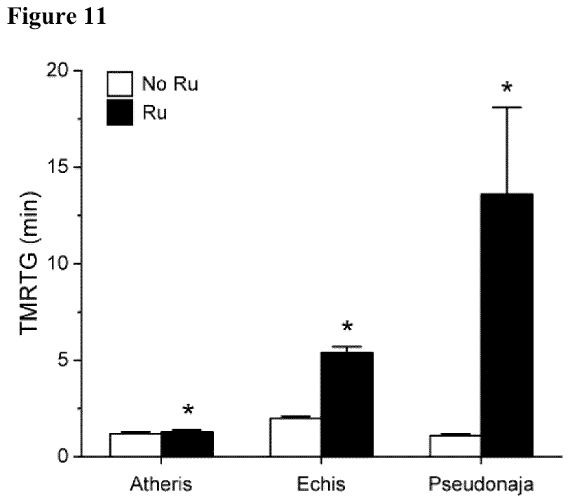

Utilizing the same general experimental approach and specific concentrations of the three venoms tested, additional aliquots of each venom was exposed to 100 μM concentrations of RuCl₃ in PBS for 5 min prior to being placed into the plasma mixture as a 1% addition (v/v). The rationale for this concentration of RuCl₃ was that it would be similar to that of the CORM-2 exposure experiments described earlier in this example (Assessment of the CO-Independent, Ru-Dependent Inhibition of CORM-2 on Procoagulant Activity of A. nitschei, E. leucogaster, and P. textilis Venoms Assessed with Thrombelastography). The results of these experiments are displayed in FIGS. 11, 12 and 13.

Unlike in the series with CORM-2 wherein the pattern of inhibition of procoagulation was very similar among the three venoms tested, there was remarkable diversity in modulation of procoagulant activity when the venoms were exposed to RuCl₃. A. nitschei venom had a very diminutive increase in TMRTG which is indicative of decreased procoagulant activity in response to RuCl₃ exposure, but a significant increase in both MRTG and TTG is consistent with an enhancement of procoagulation following RuCl₃ exposure. In contrast, E. leucogaster venom demonstrated a significant increase in TMRTG, decrease in MRTG and decrease in TTG values following RuCl₃ exposure. Lastly, P. textilis venom demonstrated significant loss of procoagulant activity after RuCl₃ exposure to a qualitatively greater extent than the other two venoms. When compared to the relatively consistent pattern and degree of procoagulant activity among the three venoms provided by CORM-2 via a presumed CO-independent/Ru-dependent mechanism, modulation of the venoms by RuCl₃ resulted in diverse changes in venom activity.

### Discussion

The first series of experiments demonstrated that the procoagulant activity of A. nitschei, E. leucogaster, and P. textilis venom was not inhibited by CO but instead by a presumed Ru-based CORM-2 radical that likely binds to venom enzyme histidine residues, evidenced by the loss of CORM-2 mediated inhibition in the presence of histidine-rich human albumin. These studies provided excess histidines to bind with reactive Ru species formed during CO release from CORM-2 by using 5% albumin (752 μM) as the solution for isolated exposures of venom to 100 μM CORM-2. Given that CORM-2 forms 70 μM of reactive Ru species during CO release from 100 μM CORM-2 (see, Motterlini, R.; et al., Circ. Res. 2002, 90, E17-E24), and that albumin has 16 histidine residues (see, Meloun, B.; et al., FEBS Lett. 1975, 58, 134-137), in the first series of experiments there was a 160:1 molar excess of histidine to react with Ru-based species. Thus, as was recently demonstrated with the same experimental approach with PLA₂ derived from Apis mellifera venom (see, Nielsen, V. G. J. Thromb. Thrombolysis 2020, 49, 100-107) and SVMP contained within mamba venom (see, Nielsen, V. G.; et al., Int. J. Mol. Sci. 2020, 21, 2082), it appears that procoagulant enzymes derived from the three venoms tested are vulnerable to inhibition by a Ru-based species formed from CORM-2.

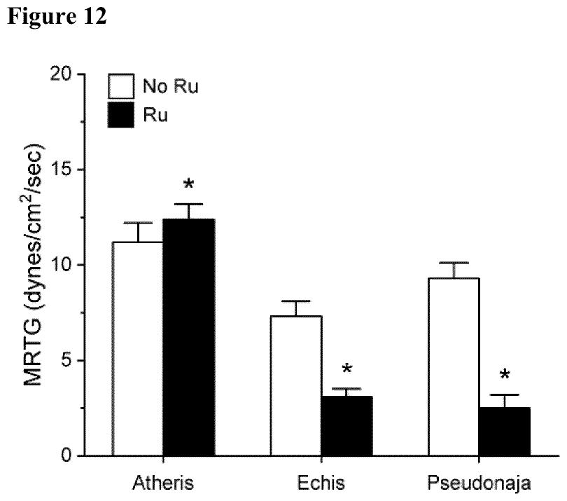

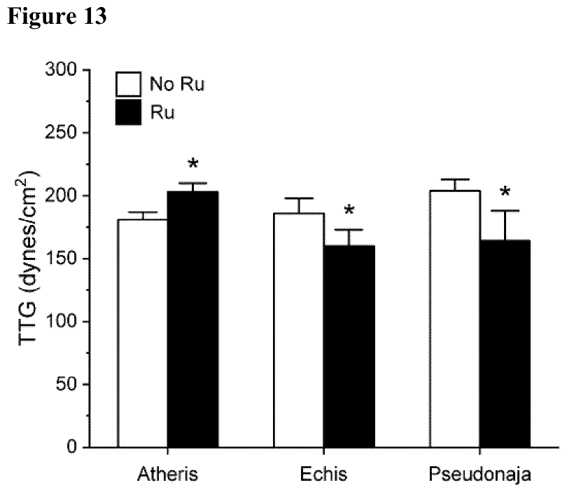

The second series of experiments provided further evidence that Ru-based molecules could modulate procoagulant snake venom enzymes, but with more variability of response to exposure to RuCl₃ compared to the CORM-2 experiments. There were three very different degrees in increase in TMRTG values in response to RuCl₃ exposure as seen in FIG. 11. In contrast to TMRTG, in the case of MRTG values it appeared that RuCl₃ exposure enhanced A. nitschei venom procoagulant activity, whereas in the cases of E. leucogaster and P. textilis venoms there was inhibition of procoagulant activity by RuCl₃ as displayed in FIG. 12. Again, as with MRTG, changes in TTG values generated by the three venoms to RuCl₃ followed the same species-specific pattern of enhanced procoagulant activity by A. nitschei venom and inhibited activity of E. leucogaster and P. textilis venoms as noted in FIG. 13. Given that RuCl₃ in PBS does enhance MRTG to a small but significant extent, the MRTG results obtained with A. nitschei venom may be indicative of venom-independent enhancement of coagulation; however, the mixed finding of small increases in TMRTG values and increase in TTG values are likely secondary to direct modulation of A. nitschei venom as there were no venom-independent effects on TMRTG and TTG by RuCl₃ (FIG. 10) consistent with these venom-mediated changes. The mechanisms responsible for differential effects on the three venoms by CORM-2 (Ru¹²) compared to RuCl₃ (Ru⁺³) remain to be defined, but when considered as a whole, the data of the present work strongly support the concept that Ru-based molecules, and not CO, are likely responsible for the CORM-2 mediated inhibition of diverse snake and lizard venoms (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79; Gessner, G.; et al., Eur. J. Pharmacol. 2017, 815, 33-41).

An unexpected finding was the procoagulant effects of RuCl₃ on human plasma that was enhanced by having the compound dissolved in PBS as seen in FIG. 10. This laboratory, using thrombelastographic methods, has already documented the effects of Fe*³ (see, Nielsen, V. G.; Pretorius, E. Blood Coagul. Fibrinolysis 2014, 25, 695-702) and Cu⁺² (see, Nielsen, V. G.; et al., J. Thromb. Thrombolysis 2018, 46, 359-364) as procoagulant and anticoagulant metals, respectively, via modulation of fibrinogen. However, neither Hg⁺² nor Pb⁺² at lethal concentrations were found to affect human plasmatic coagulation with the same thrombelastographic methods (see, Nielsen, V. G. J. Thromb. Thrombolysis 2019, 48, 697-698). Thus, the discovery that a Ru⁺³ compound (RuCl₃) at concentrations of a 1 μM affects plasmatic coagulation while a Ru⁺² compound (inactivated CORM-2 (see, Nielsen, V. G.; et al., Blood Coagul. Fibrinolysis 2009, 20, 377-380; Nielsen, V. G.; et al., Blood Coagul. Fibrinolysis 2009, 20, 448-455) needs to be at 100 μM concentrations to increase MRTG was unanticipated and interesting. The precise mechanism responsible for RuCl₃ and its PBS induced ionic species was not defined by this investigation as it is beyond its scope.

The utilization of thrombelastography to ask and answer molecular biology questions has been occurring for well over two decades, with numerous articles addressing hematological matters in several fields of investigation. It is critical to note that it is not the technique in the mechanical sense, but rather the parameters assessed and the composition of the sample analyzed that transform descriptive data that is phenomenological to parametric data that provides mechanistic insight into a focused experimental approach to testing molecular biological hypotheses. For example, the venoms assessed in this work are thrombin-generators that either activate prothrombin directly or indirectly by activating immediate precursor serine proteases in human plasmatic coagulation pathways. This feature of the venoms is best tested in a system with a relatively weak endogenous thrombin-generating scenario such as that associated with contact protein activation via interaction with the plastic surfaces of the thrombelastographic cup and pin. This allows the venom to outcompete such comparatively weak contact protein activation, permitting one the ability to assess the procoagulant activity in the presence or absence of prior isolated exposure to inhibitors or other relevant modulators. Use of the parameters TMRTG, MRTG, and TTG permits the use of parametric statistics as these expressions of clot initiation, velocity of growth, and final strength are not relatively qualitative as the unprocessed thrombelastographic tracing or nonparametric parameters such as the angle (°) or maximum amplitude (mm) (see, Ellis, T. C.; et al., Blood Coagul. Fibrinolysis 2007, 18, 45-48). Furthermore, the use of plasma, and not whole blood with intact platelet activity, simplifies the output of the experiment wherein the coagulation kinetics are dependent on the invariant fibrinogen concentration and FXIII activity that are critical as previously published (see, Nielsen, V. G.; et al., Acta Anaesthesiol. Scand. 2005, 49, 222-231; Nielsen, V. G.; Kirklin, J. K.; Hoogendoorn, H.; Ellis, T. C.; Holman, W. L. Blood Coagul. Fibrinolysis 2007, 18, 145-150). When summated, this experimental system will provide unambiguous data that is highly reproducible. The introduction of the variation of platelet concentration, variability in platelet glycoprotein IIb/IIIa receptor content, red blood cell concentration, artificially created blood flow models, etc., provide no additional mechanistic insight and instead introduce multiple confounding effects that may preclude testing the hypothesis of the present work. Similarly, utilizing standard coagulation assessments such as activated partial thromboplastin time (contact protein activation) or prothrombin time (tissue factor activated) simply introduce increased thrombin generation via activation of plasmatic contact protein and factor VII pathways, respectively—which would only compete with the venoms tested and provide no mechanistic insight. As was mentioned previously in this passage, the goal was to create an environment wherein the thrombin-generating activity of the venoms would not be confounded by any other coagulation activation. Taken as a whole, the experimental approach taken by the present investigation was designed to produce the unambiguous data presented to vigorously and conclusively test the hypothesis espoused.

In conclusion, the results described in this example determined that CORM-2 inhibited three already characterized procoagulant venoms via a CO-independent, likely Ru-based radical-dependent mechanism. Furthermore, a Ru⁺³-based ion also differentially affected the procoagulant activity of the venoms tested. With regard to utilization of Ru-based compounds as antivenoms, while the inhibitory effects on venom hemotoxic activity was inhibited by albumin in vitro at concentrations observed in vivo in the circulation, it is planned to administer such Ru-based compounds at concentrations at least 10-fold greater at the bite wound. Venom exposure to such concentrations has already been performed with neutralization in vivo in rabbits as recently noted (see, Nielsen, V. G. Basic Clin. Pharmacol. Toxicol. 2018, 122, 82-86). Furthermore, these results continue to broaden the questioning of the effects of CORM-2 being CO-based, supporting the concept that the several hundred investigations conducted over the past few decades may include situations wherein Ru-based radical activity may be responsible for the effects of CORM-2.

### Chemicals and Human Plasma

Lyophilized A. nitschei, E. leucogaster, and P. textilis venoms were originally obtained from Mtoxins (Oshkosh, WI, USA). Venoms were dissolved into calcium-free phosphate buffered saline (PBS, Millipore Sigma, Saint Louis, MO, USA) to a final 50 mg/mL concentration, aliquoted, and maintained at −80° C. The aliquots used came from the same lot published previously (see, Nielsen, V. G.; Frank, N. Biometals 2018, 31, 951-959; Nielsen, V. G.; Frank, N. Role of heme modulation in inhibition of Atheris, Atractaspis, Causus, Cerastes, Echis, and Macrovipera hemotoxic venom activity. Hum Exp. Toxicol. 2019, 38, 216-226). Dimethyl sulfoxide (DMSO), tricarbonyldichlororuthenium (II) dimer (CORM-2), and RuCl₃ were obtained from Millipore Sigma (Saint Louis, MO, USA). Human albumin solution (5% in 0.9% NaCl) was obtained from Grisfols Biologicals Inc. (Los Angeles, CA, USA). Calcium chloride (200 mM) was obtained from Haemonetics Inc. (Braintree, MA, USA). Pooled normal human plasma that was sodium citrate anticoagulated and maintained at −80° C. was obtained from George King Bio-Medical (Overland Park, KS, USA). This plasma is a commercial product collected from consented, anonymous, and compensated healthy donors by the vendor, so no further consent is needed to be obtained by end users.

### Thrombelastographic Analyses

The volumes of subsequently described plasmatic and other additives summed to a final volume of 360 μL. Samples were composed of 320 μL of plasma; 16.4 μL of PBS, 20 μL of 200 mM CaCl₂), and 3.6 μL of PBS, RuCl₃, or venom solution mixture, which were pipetted into a disposable cup in a Thrombelastograph® hemostasis system (Model 5000, Haemonetics Inc., Braintree, MA, USA) at 37° C., and then rapidly mixed by moving the cup up against and then away from the plastic pin five times. The PBS, RuCl₃, or venom solution mixtures was always the last constituent added prior to mixing and data collection. The following viscoelastic parameters described previously (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79) were measured: time to maximum rate of thrombus generation (TMRTG): this is the time interval (minutes) observed prior to maximum speed of clot growth; maximum rate of thrombus generation (MRTG): this is the maximum velocity of clot growth observed (dynes/cm²/s); and total thrombus generation (TTG, dynes/cm²), the final viscoelastic resistance observed after clot formation. Data were collected until the clot strength reached its final plateau (maximum amplitude) that was stable for 3 min.

The concentrations of venom that were used were as previously indicated in results and past manuscripts (see, Nielsen, V. G.; Frank, N. Biometals 2018, 31, 951-959; Nielsen, V. G.; Frank, N. Hum Exp. Toxicol. 2019, 38, 216-226).

### CORM-2 Addition Experiments

In experiments with CORM-2 the conditions utilized were: (1) control condition—no venom, DMSO 1% addition (v/v) in PBS; (2) V condition—venom at the concentration determined in preliminary studies, DMSO 1% addition (v/v) in PBS; (3) VC condition—venom at the concentration as in condition 2, CORM-2 1% addition in DMSO in PBS (100 μM); (4) VC+A condition—venom and CORM-2 1% addition in DMSO in 5% human albumin (100 μM final concentration). Solutions were incubated for 5 min at room temperature, and then 3.6 μL of one of these solutions was added to the plasma sample in the plastic cup.

### RuCl₃ Addition Experiments

In preliminary experiments with RuCl₃, it was determined that the final concentration of 1 μM increased MRTG. Thus, experiments wherein RuCl₃ was dissolved in either dH₂O or PBS were conducted with the final concentration of RuCl₃ in plasma being 1 or 10 μM. Data were collected until maximum amplitude was observed.

In venom exposure experiments, aliquots of venom dissolved in PBS were exposed to 100 μM RuCl₃ previously dissolved in PBS for 5 min at room temperature prior to being placed in the plasma mixture in the thrombelastographic cup as a 1% (v/v) addition. Data were collected until maximum amplitude was observed.

### Graphics and Statistical Analyses

Data are presented as mean±SD. Graphics were generated with a commercially available program (Origen2020, OrigenLab Corporation, Northampton, MA, USA). Experimental conditions were composed of n=6 replicates per condition as this provides a statistical power >0.8 with p<0.05 utilizing these techniques (see, Nielsen, V. G.; et al., Biometals 2016, 29, 913-919; Nielsen, V. G.; Losada, P. A. Basic Clin. Pharmacol. Toxicol. 2017, 120, 207-212; Nielsen, V. G.; Frank, N. J. Thromb. Thrombolysis 2019, 47, 533-539; Nielsen, V. G.; Bazzell, C. M. J. Thromb. Thrombolysis 2017, 43, 203-208; Nielsen, V. G.; Matika, R. W. Hum. Exp. Toxicol. 2017, 36, 727-733; Nielsen, V. G.; et al., Toxins 2018, 10, 322; Nielsen, V. G.; et al., Biometals 2018, 31, 51-59; Nielsen, V. G.; Frank, N. et al., Biometals 2018, 31, 951-959; Nielsen, V. G.; et al., Toxins 2019, 11, 94; Nielsen, V. G.; Frank, N. Hum Exp. Toxicol. 2019, 38, 216-226; Nielsen, V. G. J. Thromb. Thrombolysis 2019, 48, 256-262; Nielsen, V. G.; et al., Blood Coagul. Fibrinolysis 2019, 30, 379-384; Nielsen, V. G. J. Thromb. Thrombolysis 2019, 47, 73-79). A statistical program was used for one-way analyses of variance (ANOVA) comparisons between conditions, followed by Holm-Sidak post hoc analysis (SigmaPlot 14, Systat Software, Inc., San Jose, CA, USA). p<0.05 was considered significant.

### Example IV

This example describes the modulation of diverse procoagulant venom activities by combinations of platinoid compounds.

### Assessment of CORM-2 and RuCl₃, Separately and in Combination, on the Procoagulant Activity of B. Moojeni, C. Rhodostoma, E. Leucogaster and O. Microlepidotus Venoms

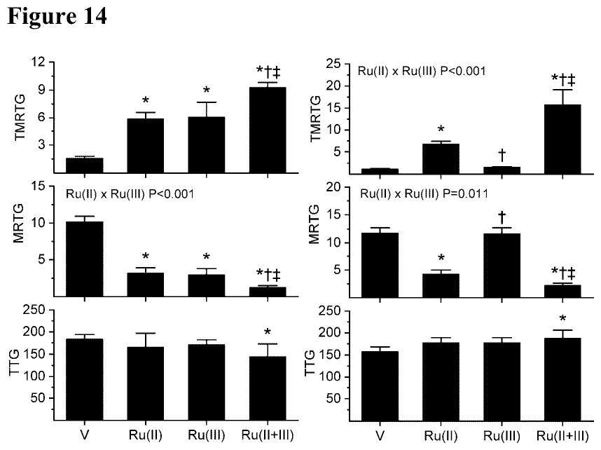

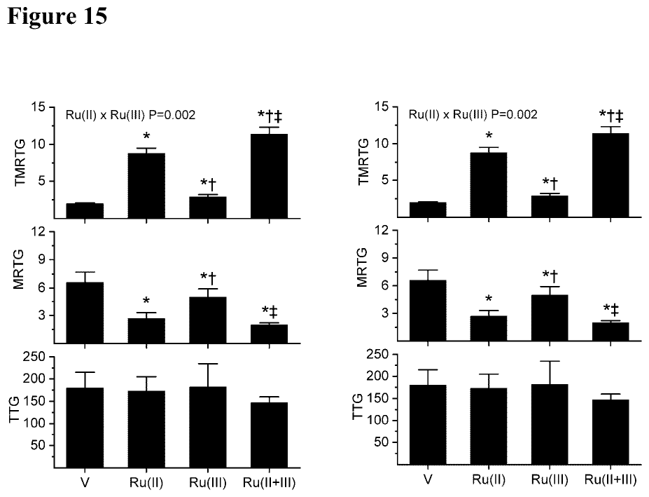

The subsequent results were obtained using concentrations or fractions thereof of the aforementioned venoms previously published using these methods (see, Nielsen, V. G.; Bazzell, C. M. J Thromb Thrombolysis 2017, 43, 203-208; Nielsen, V. G.; Frank, N.; Matika, R. W. Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94; Suntravat, M.; et al., Biometals 2018, 31, 585-593; Nielsen, V. G. J Thromb Thrombolysis 2019, 47, 73-79; Nielsen, V. G.; Frank, J Thromb Thrombolysis 2019, 47, 533-539); specifically, B. moojeni venom had a final concentration of 2 μg/ml, C. rhodostoma a venom concentration of 5 μg/ml, E. leucogaster venom a venom concentration of 1 μg/ml and O. microlepidotus venom concentration of 1 μg/ml in the plasma mixtures tested. Venom concentrations were selected on a performance basis wherein the activation of coagulation by the venom statistically exceeded the activation observed by contact activation with thrombelastographic cup and pin contact with plasma as previously described (see, Nielsen, V. G.; Bazzell, C. M. J Thromb Thrombolysis 2017, 43, 203-208; Nielsen, V. G.; Frank, N.; Matika, R. W. Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94; Suntravat, M.; et al., Biometals 2018, 31, 585-593; Nielsen, V. G. J Thromb Thrombolysis 2019, 47, 73-79; Nielsen, V. G.; Frank, J Thromb Thrombolysis 2019, 47, 533-539). All venom solutions without or with chemical additions in isolation were added as a 1% addition to the plasma mix used in our thrombelastographic system (see, Nielsen, V. G.; Bazzell, C. M. J Thromb Thrombolysis 2017, 43, 203-208; Nielsen, V. G.; Frank, N.; Matika, R. W. Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94; Suntravat, M.; et al., Biometals 2018, 31, 585-593; Nielsen, V. G. J Thromb Thrombolysis 2019, 47, 73-79; Nielsen, V. G.; Frank, J Thromb Thrombolysis 2019, 47, 533-539). This dilution is critical, as it reduces the concentration of CORM-2 to 1 μM, a concentration at which this compound does not affect coagulation kinetics (see, Nielsen, V. G.; Frank, N.; Afshar, S. Toxins (Basel) 2019, 11, E94). Also, it has been demonstrated that concentrations of RuCl₃ at or below 1 μM does not significantly affect human plasmatic coagulation, meaning that exposure of venom to up to 100 μM in isolation in this system should not affect interpretation of changes in venom procoagulant activity (see, Nielsen, V. G. J Thromb Thrombolysis 2021, doi: 10.1007/s11239-020-02373-4). With regard to the concentrations of CORM-2 and RuCl₃ used in the isolated exposures, they were as follows: B. moojeni venom was exposed to 0-1 mM CORM-2 and 0-100 μM RuCl₃; C. rhodostoma venom was exposed to 0-50 μM CORM-2 and 0-50 RuCl₃; E. leucogaster venom was exposed to 0-100 μM CORM-2 and 0-100 μM RuCl₃; and, O. microlepidotus venom to 0-100 μM CORM-2 and 0-100 μM RuCl₃. Lastly, the thrombelastographic model utilized describes coagulation kinetics with the following three variables: time to maximum thrombus generation (TMRTG, minutes—a measure of time to onset of coagulation), maximum rate of thrombus generation (MRTG, dynes/cm²/sec—a measure of the velocity of clot growth) and total thrombus generation (TTG, dynes/cm²—a measure of clot strength). The results of exposing the four venoms to CORM-2 and RuCl₃ separately or in combination are displayed in the following FIGS. 14 and 15.

As seen in FIG. 14 in the left panels, exposure of B. moojeni venom to 1 mM CORM-2 (depicted as Ru(II)) resulted in a significant increase in TMRTG and decrease in MRTG values compared to CORM-2 naïve venom, indicative of inhibition of procoagulant activity. In contrast, exposure of B. moojeni venom to 100 μM RuCl₃ (depicted as Ru(III)) did not significantly diminish procoagulant activity in this dataset. When B. moojeni venom was exposed to the combination of CORM-2 and RuCl₃, TMRTG values were far more increased and MRTG values decreased compared to all other conditions, with the inhibition of the procoagulant activity due to significant interaction of the two Ru-based compounds. Lastly, there were no effects of the Ru-based compounds on the final clot strength generated by the procoagulant activity of B. moojeni venom except in the condition wherein both were present, resulting in TTG values significantly greater than the condition wherein venom alone was present.

With regard to results obtained with C. rhodostoma venom, these are displayed in the right panels of FIG. 14. Exposure of C. rhodostoma venom to 50 μM CORM-2 resulted in a significant increase in TMRTG and decrease in MRTG values compared to CORM-2 naïve venom, revealing procoagulant activity inhibition. Similarly, exposure of C. rhodostoma venom to 50 μM RuCl₃ significantly diminished procoagulant activity, evidenced by an increase in TMRTG values and a decrease in MRTG values compared to venom not exposed to RuCl₃. When C. rhodostoma venom was exposed to the combination of CORM-2 and RuCl₃, TMRTG values were far more increased and MRTG values decreased compared to all other conditions, with the inhibition of the procoagulant activity assessed by changes in MRTG due to a significant interaction of the two Ru-based compounds. Lastly, there was a significant decrease in TTG values when the venom was exposed to both CORM-2 and RuCl₃ compared to venom exposed to neither compound. In summary, these diverse venoms displayed an enhanced inhibition of procoagulant activity following exposure to the combination of CORM-2 and RuCl₃ compared to separate exposures of either compound.

As for the next two venoms tested, the results obtained with E. leucogaster venom and O. microlepidotus venom are displayed in the left and right panels of FIG. 15. With regard to E. leucogaster venom, exposure to 100 μM CORM-2 resulted in a significant increase in TMRTG and decrease in MRTG values compared to CORM-2 naïve venom, demonstrating procoagulant activity inhibition. Similarly, exposure of E. leucogaster venom to 100 μM RuCl₃ significantly diminished procoagulant activity, evidenced by an increase in TMRTG values and a decrease in MRTG values compared to venom not exposed to RuCl₃. However, the inhibition of procoagulant activity was significantly less than that observed with CORM-2. When E. leucogaster venom was exposed to the combination of CORM-2 and RuCl₃, TMRTG values were significantly more increased compared to all other conditions. Inhibition of venom activity assessed by changes in TMRTG due to a significant interaction of the two Ru-based compounds was also present. Further, MRTG values were significantly decreased compared to venom without exposures and venom exposed to RuCl₃ but not different from venom exposed only to CORM-2. Lastly, there were no significant changes in TTG values between the conditions.

The results obtained with O. microlepidotus venom are displayed in the right panels of FIG. 15. Exposure of this venom to 100 μM CORM-2 resulted in a significant increase in TMRTG, decrease in MRTG, and decrease in TTG values compared to CORM-2 naïve venom, demonstrating procoagulant activity inhibition. In sharp contrast, exposure of O. microlepidotus venom to 100 μM RuCl₃ did not significantly affect procoagulant activity. Lastly, when O. microlepidotus venom was exposed to the combination of CORM-2 and RuCl₃, the decrease in procoagulant activity was not significantly different from venom exposed to CORM-2 alone but significantly more inhibited than venom without exposure to any compounds or exposed to RuCl₃.

These series of experiments with these four diverse venoms demonstrated very different patterns of inhibition by CORM-2, RuCl₃, or the combination of these two compounds.

Assessment of CORM-3 and RuCl₃, Separately and in Combination, on the Procoagulant Activity of B. Moojeni, C. Rhodostoma, P. Textilis and H. suspectum Venoms.

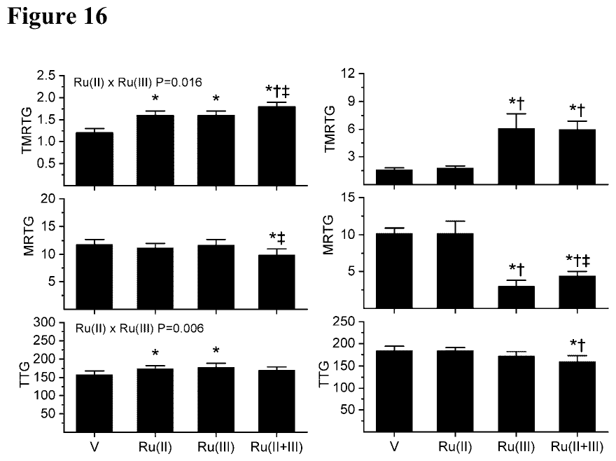

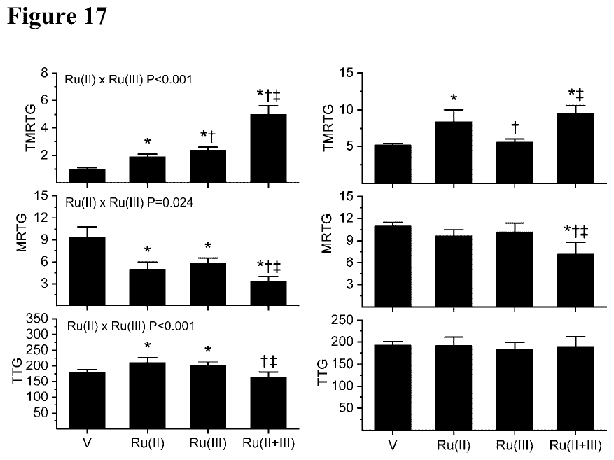

For this third series of experiments, B. moojeni venom had a final plasma sample concentration of 2 μg/ml, C. rhodostoma a venom concentration of 5 μg/ml, P. textilis a venom concentration of 0.1 μg/ml, and H. suspectum venom a concentration of 10 μg/ml. All other aspects of the plasma mix used are similar to that of the previous series. The concentrations of CORM-2 and RuCl₃ used in the isolated exposures were as follows: B. moojeni venom was exposed to 0-1 mM CORM-3 and 0-100 μM RuCl₃; C. rhodostoma venom was exposed to 0-50 μM CORM-3 and 0-50 RuCl₃; P. textilis venom was exposed to 0-100 μM CORM-2 and 0-100 μM RuCl₃; and, H. suspectum venom to 0-1 mM CORM-2 and 0-100 μM RuCl₃. The results of exposing the four venoms to CORM-3 and RuCl₃ separately or in combination are displayed in the following FIGS. 16 and 17.

As seen in FIG. 16 in the left panels, exposure of B. moojeni venom to 1 mM CORM-3 or 100 μM RuCl₃ resulted in a significant increase in TMRTG compared to additive naïve venom, indicative of inhibition of procoagulant activity. In contrast, exposure of B. moojeni venom to CORM-3 or RuCl₃ did not significantly change MRTG values. Exposure to both Ru-based compounds significantly increased TMRTG values compared to all other conditions, and MRTG values were decreased compared to venom without exposure to additives or exposure to RuCl₃. TTG values were significantly increased by either Ru-based compound but TTG values decreased to values observed with venom not exposed to additives. This change in TTG values resulted in a significant interaction between CORM-3 and RuCl₃.

The results obtained with C. rhodostoma venom are displayed in the right panels of FIG. 16. Exposure of this venom to 100 μM CORM-3 resulted in no significant effect on procoagulant activity. In sharp contrast, exposure of C. rhodostoma venom to 100 μM RuCl₃ resulted in a significant increase in TMRTG and decrease in MRTG compared to RuCl₃ naïve venom or CORM-3 exposed venom but not different from RuCl₃ exposed venom. With regard to MRTG values, the combination of CORM-3 and RuCl₃ resulted in values significantly smaller than RuCl₃ naïve venom or CORM-3 exposed venom; however, MRTG values under these conditions were significantly greater than that associated with venom exposed to RuCl₃ alone. Lastly, when C. rhodostoma venom was exposed to the combination of CORM-3 and RuCl₃, TTG values were significantly smaller than those observed in RuCl₃ naïve venom or CORM-3 exposed venom samples.

Data obtained from experiments performed with P. textilis venom and H. suspectum venom are depicted in the left and right panels of FIG. 5, respectively. P. textilis venom exposed to 100 μM CORM-3 or 100 μM RuCl₃ resulted in a significant increase in TMRTG, decrease in MRTG, and increase in TTG values compared to additive naïve venom. Further, when exposed to both CORM-3 and RuCl₃, TMRTG values significantly larger than and MRTG values significantly smaller than the other three conditions were observed. In contrast, TTG values observed after venom was exposed to both CORM-3 and RuCl₃ were significantly smaller than in samples with venom exposed to either Ru-based compound separately. Lastly, CORM-3 and RuCl₃ demonstrated significant interactions on venom activity as seen in the two-way ANOVA analyses.

Data obtained from experiments performed with H. suspectum venom are presented in the right panel of FIG. 17. Exposure of this venom to CORM-3 resulted in a significant increase in TMRTG values in plasma but no change in either MRTG or TTG values. Exposure of H. suspectum venom to RuCl₃ resulted in no significant changes in any of the coagulation kinetic parameters. However, exposure to both CORM-3 and RuCl₃ resulted in TMRTG values significantly greater than venom not exposed to additives or venom exposed to RuCl₃. In contrast, MRTG values were significantly decreased by the combination of CORM-3 and RuCl₃ compared to all other conditions. Not changes in TTG were noted between the conditions. In summary, the exposure of H. suspectum venom to CORM-3 and RuCl₃ in various combinations resulted in significant but quantitatively small inhibition of procoagulant activity.

This series of experiments demonstrated a diverse response to CORM-3 mediated inhibition compared to CORM-2 attenuation of activity with four very different venoms.

Assessment of CORM-2 and Carboplatin, Separately and in Combination, on the Procoagulant Activity of B. moojeni and C. rhodostoma Venoms.

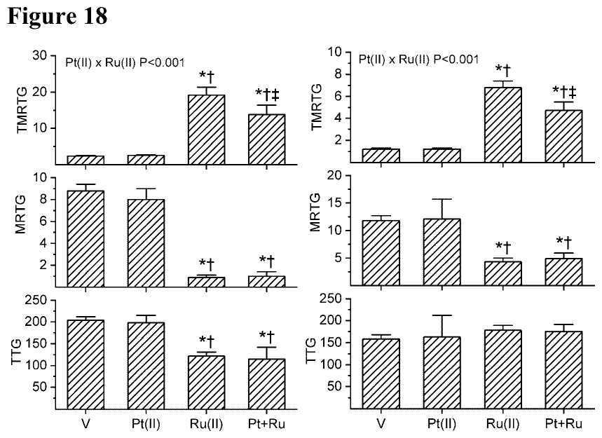

As with the previous experiments, B. moojeni venom had a final concentration of 2 μg/ml and C. rhodostoma a venom concentration of 5 μg/ml. All other aspects of the plasma mix used are similar to that of the two previous series except that the venoms were exposed to different combinations of carboplatin and CORM-2. The concentrations of carboplatin and CORM-2 used in the isolated exposures were as follows: B. moojeni venom was exposed to 0-100 μM carboplatin (depicted as Pt(II)) and 0-1 mM CORM-2; and, C. rhodostoma venom was exposed to 0-100 μM carboplatin and 100 μM CORM-2. The results of exposing these two venoms to carboplatin and CORM-2 separately or in combination are displayed in the following FIG. 18, with data generated with B. moojeni venom in the left panels and data obtained with C. rhodostoma venom in the right panels.

As seen in FIG. 18 in the left panels, exposure of B. moojeni venom to carboplatin resulted in no significant change in any of the coagulation kinetic parameters compared to samples with venom without additives. CORM-2 exposure resulted in significantly increased TMRTG and decreased MRTG values compared to CORM-2 naïve or carboplatin exposed venom samples. When carboplatin and CORM-2 were combined, TMRTG values were significantly different from the other three conditions with the important observation that the addition of carboplatin to CORM-2 decreased TMRTG values compared to samples with CORM-2 exposure. The interaction between carboplatin and CORM-2 was significant for TMRTG values, with carboplatin opposing CORM-2 mediated inhibition of the procoagulant activity of this venom. Lastly, no statistically significant changes in TTG values were noted between the four conditions.

The results obtained with C. rhodostoma venom are displayed in the right panels of FIG. 18. Exposure of this venom to 100 μM carboplatin resulted in no significant effect on procoagulant activity. In sharp contrast, exposure of C. rhodostoma venom to 100 μM CORM-2 resulted in a significant increase in TMRTG, decrease in MRTG and decrease in TTG values compared to CORM-2 naïve venom or carboplatin. Exposure of this venom to the combination of carboplatin and CORM-2 and CORM-2 resulted in TMRTG values significantly different from the values other three conditions with the important finding that the addition of carboplatin to CORM-2 decreased TMRTG values compared to samples with CORM-2 exposure. Aside from this singular difference in TMRTG values, there was no significant differences in MRTG and TTG values between venom exposed to CORM-2 alone or the combination of carboplatin and CORM-2. As with B. moojeni venom, the interaction between carboplatin and CORM-2 was significant for TMRTG values, with carboplatin opposing CORM-2 mediated inhibition of the procoagulant activity of C. rhodostoma venom.

In conclusion, carboplatin did not demonstrate any detectable effect on the procoagulant activity of these two venoms, but this compound did in some way partially block the inhibitory effect of CORM-2 on increasing TMRTG values, thus delaying the initiation of clot formation.

### Discussion

The experiments described in Example IV succeeded in capturing unique observations regarding the effects of four platinoid compounds with different valences on diverse procoagulant venom activities. To be sure, the venoms contained metalloproteinases, serine proteases, kallikrein-like enzymes, Factor X-like enzymes, and/or Factor V-like activities (see, Aguiar, W. D. S.; et al., PLoS One 2019, 14; Tang, E. L.; et al., J Proteomics 2016, 148, 44-56; Patra, A.; et al., Sci Rep 2017, 7, 17119; Yamada, D.; Morita, T. Thromb Res 1999, 94, 221-226; Chen, Y. L.; Tsai, I. H. Biochemistry 1996, 35, 5264-52671; Koludarov, I.; et al., Toxins (Basel) 2014, 6, 3582-3595; Sanggaard, K. W.; et al., J Proteomics 2015, 117, 1-11; Herrera, M.; et al., J Proteomics 2012, 75, 2128-2140; McCleary, R. J.; et al., J Proteomics 2016, 144, 51-62), and the vipers and one lizard chosen evolved in geographically diverse areas of the world. This selection of compounds and venoms permitted remarkably different results to be documented that provide molecular insight into the complex interactions modifying procoagulant activity. For clarity, the various patterns of interaction of the platinoids utilized with venoms will be considered in the order of experimentation as subsequently presented.

With regard to the interaction of CORM-2 and RuCl₃, B. moojeni inhibition was possibly secondary to separate molecular interactions of the enzyme(s) affected by the two Ru-based compounds, with a relatively silent kinetic interaction by RuCl₃ that only became important when CORM-2 was introduced. As for C. rhodostoma venom, it appeared that both Ru-based compounds individually inhibited procoagulant activity to an equivalent extent, and when combined, significantly inhibited activity more than when only one inhibitor was present. In the case of E. leucogaster venom procoagulant activity, CORM-2 was a more significant inhibitor than RuCl₃, but when combined, inhibition was somewhat greater than when the venom was exposed to CORM-2 alone. Lastly, O. microlepidotus venom procoagulant activity was only inhibited by CORM-2, with the presence of RuCl₃ not making any difference in activity without or with CORM-2 presence. Considered as a whole, the results point to potential diversity in binding site by CORM-2 and RuCl₃, associated with equally unpredictable inhibitory effect.

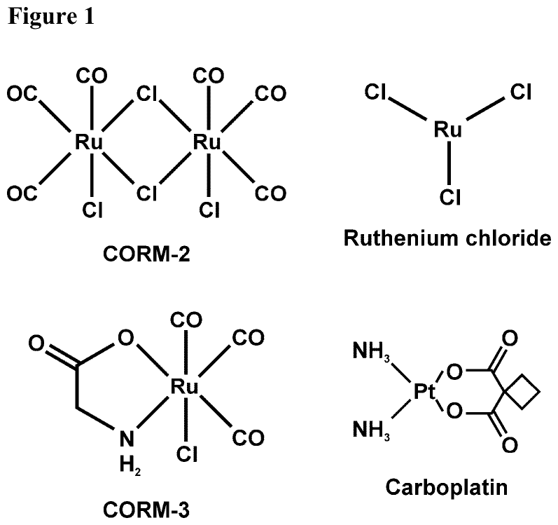

The experiments involving venom exposures to CORM-3 and RuCl₃ also revealed diverse patterns of procoagulant activity inhibition. In the case of B. moojeni venom, insignificant inhibition by both CORM-3 and RuCl₃ were noted (FIG. 16), but the inhibitory effects were far more diminutive than that observed with CORM-2 (FIG. 1). The reason that the procoagulant activity of venom after RuCl₃ exposure was significantly different from venom not exposed to this compound in FIG. 16 but not in FIG. 1 is a statistical phenomenon—the mean values and variance of the other conditions in FIG. 1 overshadowed the condition of RuCl3-exposed venom but not so in FIG. 16. Nevertheless, the effects of RuCl₃ on this venom was quantitatively very small. As for C. rhodostoma venom exposed to CORM-3 and RuCl₃, CORM-3 had no discernable effect on procoagulant activity, and when combined with RuCl₃, it appeared that CORM-3 partially antagonized RuCl₃-mediated inhibition of procoagulant activity based on an increase in MRTG values compared to venom samples exposed to RuCl₃ alone (FIG. 16). With regard to P. textilis venom procoagulant activity, CORM-3 and RuCl₃ had equivalent inhibitory effects, with the combination of the two Ru-based compounds significantly interacting and significantly increasing inhibition of activity. Finally, as for H. suspectum venom procoagulant activity, only the combination of CORM-3 and RuCl₃ exerted meaningful inhibition of activity. In summary, unlike CORM-2, CORM-3 was unpredictably far less potent as a direct inhibitor of procoagulant activity in some cases, and unpredictably enhanced or partially inhibited RuCl₃-mediated inhibition of procoagulant activity.

Experimentation involving carboplatin and CORM-2 were in some ways the most fascinating. Simply put, carboplatin by itself had no detectable effects on the procoagulant activity of B. moojeni and C. rhodostoma venom; however, carboplatin exposure significantly antagonized CORM-2 mediated inhibition of venom procoagulant activity as evidenced by decreased TMRTG values (FIG. 18). While at face value it seems simple enough to imagine a competition between carboplatin and CORM-2 on a common molecular site of similar enzymes, it is far more difficult to explain with such a paradigm why MRTG values did not change as well. As a rule, increases or decreases in thrombin generation are accompanied by concordant decreases or increases in TMRTG values and increases or decreases in MRTG values, respectively. Taken as a whole, while it is clear that a Pt-based compound appears to partially antagonize a Ru-based compound mediated inhibition of procoagulant activity, the precise molecular explanation for the coagulation kinetic changes observed remains to be elucidated.

In conclusion, the experiments described in Example IV demonstrated that hereto unappreciated binding sites on procoagulant enzymes within diverse venoms with complex proteomes may be vulnerable to inhibition of activity by a variety of Ru-based compounds with different valences separately or as a formulation. Further, a Pt-based compound was found to antagonize Ru-based compound mediated inhibition of the procoagulant activity of diverse venoms. These observations provide molecular insight into the potentially multiple sites present on such procoagulant enzymes that may be therapeutic targets when designing small molecular weight antivenom molecules. Future investigation is justified to determine the differential response of hemostatically active venoms (e.g., procoagulant, anticoagulant, neurotoxic) to Ru-based compounds of multiple valences and molecular size, in isolation and in formulations of two or more compounds.

### Chemicals and Human Plasma.

Calcium-free phosphate buffered saline (PBS), CORM-2, CORM-3, ruthenium chloride and carboplatin were obtained from Millipore Sigma (Saint Louis, MO, USA). Venoms dissolved in PBS (50 mg/ml) were obtained from archived, never thawed aliquots maintained at −80° C. in the laboratory that were used in previous investigations (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94; Nielsen, V. G Int J Mol Sci 2020, 21, 2970; Nielsen, V. G.; Frank, N. J Thromb Thrombolysis 2019, 47, 533-539). Bothrops moojeni and Calloselasma rhodostoma venoms were obtained originally from the National Natural Toxins Research Center at Texas A&M University (Kingsville, TX, USA). Additionally, Echis leucogaster, Heloderma suspectum, Oxyuranus microlepidotus and Pseudonaja textilis venoms were originally purchased from Mtoxins (Oshkosh, WI, USA). Calcium chloride (200 mM) was obtained from Haemonetics Inc., Braintree, MA, USA. Pooled normal human plasma (George King Bio-Medical, Overland Park, KS, USA) that was sodium citrate anticoagulated and maintained at −80° C. was used.

### Thrombelastographic Analyses.

The volumes of subsequently described plasmatic and other additives summed to a final volume of 360 μl. Samples were composed of 320 μl of plasma; 16.4 μl of PBS, 20 μl of 200 mM CaCl2), and 3.6 μl of PBS or venom mixture, which were pipetted into a disposable cup in a Thrombelastograph® hemostasis system (Model 5000, Haemonetics Inc., Braintree, MA, USA) at 37° C., and then rapidly mixed by moving the cup up against and then away from the plastic pin five times. The following viscoelastic parameters described previously (see, Nielsen, V. G. J Thromb Thrombolysis 2019, 47, 73-79; Gessner, G.; et al., Eur J Pharmacol 2017, 815, 33-41; Nielsen, V. G.; Wagner, M. T.; Frank, N. Int J Mol Sci 2020, 21, 2082; Lazić, D.; et al., Dalton Trans 2016, 45, 4633; Hanif, M.; et al., ChemPlusChem 2017, 82, 841-847) were measured: time to maximum rate of thrombus generation (TMRTG): this is the time interval (minutes) observed prior to maximum speed of clot growth; maximum rate of thrombus generation (MRTG): this is the maximum velocity of clot growth observed (dynes/cm2/second); and total thrombus generation (TTG, dynes/cm2), the final viscoelastic resistance observed after clot formation. Data were collected until a stable maximum amplitude was observed with minimal change for 3 minutes as determined by the software.

### Exposures of Venoms to CORM-2, CORM-3, RuCl₃ and Carboplatin.

A selection of venoms was exposed to CORM-2 concentrations (or fractions thereof) demonstrated to inhibit procoagulant activity and placed into plasma at the final venom concentrations previously used in this plasma based, thrombelastographic system (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94). Indicated venoms were also exposed to CORM-3, RuCl₃ and carboplatin in various combinations subsequently presented. The specific exposures for each venom are as follows.

B. moojeni. This venom was exposed to 0 or 1 mM CORM-2 or CORM-3 in the presence of 0 or 100 μM RuCl₃ in PBS for at least 5 minutes at room temperature prior to placement into plasma followed immediately with commencement of thrombelastographic analysis. This venom was also exposed to 0 or 1 mM CORM-2 in the presence of 0 or 100 μM carboplatin in another series of experiments. The final concentration of this venom in plasma was 2 μg/ml.

C. rhodostoma. This venom was exposed to 0 or 50 μM CORM-2 or CORM-3 in the presence of 0 or 50 μM RuCl₃ in PBS for at least 5 minutes at room temperature prior to placement into plasma followed immediately with commencement of thrombelastographic analysis. This venom was also exposed to 0 or 100 μM CORM-2 in the presence of 0 or 100 μM carboplatin in another series of experiments. The final concentration of this venom in plasma was 5 μg/ml.

E. leucogaster. This venom was exposed to 0 or 100 μM CORM-2 in the presence of 0 or 100 μM RuCl₃ in PBS for at least 5 minutes at room temperature prior to placement into plasma followed immediately with commencement of thrombelastographic analysis. The final concentration of this venom in plasma was 1 μg/ml.

O. microlepidotus. This venom was exposed to 0 or 100 μM CORM-2 in the presence of 0 or 100 μM RuCl₃ in PBS for at least 5 minutes at room temperature prior to placement into plasma followed immediately with commencement of thrombelastographic analysis. The final concentration of this venom in plasma was 1 μg/ml.

P. textilis. This venom was exposed to 0 or 100 μM CORM-3 in the presence of 0 or 100 μM RuCl₃ in PBS for at least 5 minutes at room temperature prior to placement into plasma followed immediately with commencement of thrombelastographic analysis. The final concentration of this venom in plasma was 0.1 μg/ml.

H. suspectum. This venom was exposed to 0 or 1 mM CORM-3 in the presence of 0 or 100 μM RuCl₃ in PBS for at least 5 minutes at room temperature prior to placement into plasma followed immediately with commencement of thrombelastographic analysis. The final concentration of this venom in plasma was 10 μg/ml.

Given the aforementioned, the experimental conditions utilized were: 1) V condition—venom in PBS; 2) Ru(II) condition—venom exposed to CORM-2 or CORM-3; 3) Ru(III) condition—venom exposed to RuCl₃; 4) Ru(II+III) condition—venom exposed to CORM-2 or CORM-3 and RuCl₃ simultaneously; 5) Pt(II) condition—venom exposed to carboplatin; and 6) Pt+Ru condition—venom exposed to carboplatin and CORM-2. After the 5 minute period at room temperature, 3.6 μl of one of these solutions was added to the plasma sample in the plastic thrombelastograph cup.

### Statistical Analyses and Graphics.

Data are presented as mean±SD. Graphics were generated with a commercially available program (Origen2020b, OrigenLab Corporation, Northampton, MA, USA). Experimental conditions were composed of n=6 replicates per condition as this provides a statistical power >0.8 with P<0.05 utilizing these techniques (see, Nielsen, V. G.; et al., J Thromb Thrombolysis 2017, 43, 203-208; Nielsen, V. G.; et al., Biometals 2018, 31, 51-59; Nielsen, V. G.; Frank, N. Hum Exp Toxicol 2019, 38, 216-226; Nielsen, V. G.; Frank, N.; Afshar, S. Toxins (Basel) 2019, 11, E94). A statistical program was used for one-way analyses of variance (ANOVA) comparisons between conditions, followed by Holm-Sidak post hoc analysis. Additional analysis with two-way ANOVA was performed to detect significant interactions between CORM-2 and RuCl₃, CORM-3 and RuCl₃, and CORM-2 and carboplatin regarding changes in venom procoagulant activity. All analyses were performed with commercial software (SigmaPlot 14, Systat Software, Inc., San Jose, CA, USA). P<0.05 was considered significant.

### Example V

This example describes experiments demonstrating that ruthenium chloride inhibits the anticoagulant activity of the phospholipase A₂-dependent neurotoxin of Mojave Rattlesnake Type A venom.

A unique nexus exists between coagulation and neurotoxicity that permits the assessment of the activity of destructive presynaptic phospholipase A₂ (PLA₂) enzymatic activity. This connection is that these enzymes can catalyze critical plasma phospholipids required for thrombin generation that resemble phospholipids in the neuromuscular junction, rendering them in vitro anticoagulants as have been documented via thrombelastography in recent years by several of investigators (see, Nielsen V G (2019) J Thromb Thrombolysis 48, 256-262; Nielsen V G (2020) J Thromb Thrombolysis 49, 100-107; Dashevsky D, et al., (2021) Toxicol Lett 337, 91-97). The specificity of the PLA₂-mediated anticoagulant activity of these venoms has been confirmed by using PLA₂ inhibitors to eliminate anticoagulant effects (see, Nielsen V G (2019). J Thromb Thrombolysis 48, 256-262; Dashevsky D, et al., (2021) Toxicol Lett 337, 91-97) or by outcompeting the enzyme with the addition of phospholipids to the plasma sample (Dashevsky D, et al., (2021) Toxicol Lett 337, 91-97). Critically, administering some of these very same PLA₂ inhibitors to mice (see, Lewin M R, et al., (2018) Toxins (Basel) 10, 380) or swine (see, Lewin M R, et al., (2018) Toxins (Basel). 10, 479) in vivo prevented neurotoxin mediated apneic death. Consequently, it is possible to make the conceptual leap that inhibition of PLA₂-mediated anticoagulant activity in vitro could be associated with loss of in vivo lethality, allowing preclinical evaluation of PLA₂ inhibitors using a thrombelastographic, coagulation kinetic bioassay.

One such novel PLA₂ inhibitor is the ruthenium (Ru) containing compound CORM-2, which has been demonstrated to inhibit the anticoagulant activity of snake venoms known to contain PLA₂ activity (see, Nielsen V G (2019) J Thromb Thrombolysis 48, 256-262; Nielsen V G, Frank N, Matika R W (2018) Biometals 31, 51-59; Nielsen V G, Frank N, Turchioe B J (2019) Blood Coagul Fibrinolysis 30, 379-384). While originally these works posited that carbon monoxide (CO) released from CORM-2 were responsible for inhibition of anticoagulant activity, research has subsequently demonstrated that a likely short-lived Ru-based species derived from CORM-2 is responsible for inhibition of both anticoagulant and procoagulant snake venom enzymes (see, Nielsen V G, Wagner M T, Frank N (2020) Int J Mol Sci 21, 2082; Nielsen V G (2020) Int J Mol Sci 21, 2970). Lastly, it was demonstrated that ruthenium chloride (RuCl3) forms a phosphate associated ion (not radical) that could inhibit some, but not all, snake venom procoagulant activities (see, Nielsen V G (2020) Int J Mol Sci 21, 2970). Thus, it could be possible that RuCl₃ under the proper conditions could inhibit snake venom PLA₂ anticoagulant activity.

Therefore, a purpose of the following experiments was to determine if RuCl₃ could inhibit a well-characterized, PLA₂-based neurotoxin. The neurotoxin chosen was Mojave toxin, found in Mojave rattlesnake venom type A, which is dependent on intact PLA₂ activity for its lethal effects as recently reviewed (see, Nielsen V G (2019) J Thromb Thrombolysis 48, 256-262). Critically, this venom is devoid of proteolytic activity; therefore, all anticoagulant effect noted is secondary to PLA₂ activity on plasmatic phospholipids as recently noted (see, Nielsen V G (2019) J Thromb Thrombolysis 48, 256-262).

With regard to materials and methods, Mojave rattlesnake (Crotalus scutulatus scutulatus) venom type A was originally obtained from the National Natural Toxins Research Center (NNTRC) located at Texas A&M University-Kingsville, Kingsville, TX, U.S.A. A previously unthawed aliquot of this venom used in a previous work (see, Nielsen V G (2019) J Thromb Thrombolysis 48, 256-262) was dissolved in calcium-free phosphate buffered saline (PBS, Sigma-Aldrich, Saint Louis, MO, USA) at a concentration of 50 mg/ml, and had been stored at −80° C. RuCl₃ as obtained from Sigma-Aldrich, Saint Louis, MO, USA. Calcium chloride (CaCl₂), 200 mM) was obtained from Haemonetics Inc., Braintree, MA, USA. Lastly, pooled normal human plasma (George King Bio-Medical, Overland Park, KS, USA) anticoagulated with sodium citrate (9 parts blood to 1 part 0.105M sodium citrate) stored at −80° C. was utilized in all subsequently described experiments.

Specific volumes of subsequently described plasma and chemical additives varied by condition but summated to 360 μl. Sample composition consisted of 320 μl of plasma; 16.4 μl of PBS, 20 μl of 200 mM CaCl₂), and 3.6 μl of one of five subsequently described solutions, which were placed into a disposable cup in a computer-controlled Thrombelastograph® hemostasis system (Model 5000, Haemonetics Inc., Braintree, MA, USA) at 37° C., and then rapidly mixed by moving the cup up against and then away from the plastic pin five times before leaving the mixture between the cup and pin. The following elastic modulus-based parameters previously described (see, Nielsen V G (2019) J Thromb Thrombolysis 48, 256-262; Nielsen V G, Frank N, Matika R W (2018) Biometals 31, 51-59; Nielsen V G, Frank N, Turchioe B J (2019) Blood Coagul Fibrinolysis 30, 379-384; Nielsen V G (2021) J Thromb Thrombolysis, doi: 10.1007/s11239-020-02373-4) were determined: time to maximum rate of thrombus generation (TMRTG): this is the time interval (minutes) observed prior to maximum speed of clot growth; maximum rate of thrombus generation (MRTG): this is the maximum velocity of clot growth observed (dynes/cm²/second); and total thrombus generation (TTG, dynes/cm²), the final viscoelastic resistance observed after clot formation. Data were collected until maximum amplitude was reached as determined by the software of the thrombelastographic system.

Experiments involving plasma exposure to RuCl₃ and venom utilized the following five experimental conditions: 1) control condition (C)—no venom, 1% addition of PBS(v/v) to plasma; 2) RuCl₃ in water condition (R/W)—no venom, 1% addition (v/v) of 100 μM RuCl₃ dissolved in water to plasma (final concentration 1 μM); 3) RuCl₃ in PBS condition (R/P)—no venom, 1% addition (v/v) of 100 μM RuCl₃ dissolved in PBS to plasma (final concentration 1 μM); 4) venom condition (V)—venom addition (v/v, 125 ng/ml final concentration as previously reported [1]) in PBS to plasma; and, 5) venom exposed to RuCl₃ dissolved in PBS condition (V+R/P)—venom and 100 μM RuCl₃ 1% addition (v/v) in PBS to plasma. All the solutions used for these five conditions to be placed in plasma remained at room temperature for five minutes prior to addition. The purpose of comparing condition 2 to condition 3 was to determine if RuCl₃ dissolved in PBS could exert a procoagulant effect at this small concentration as a 50-fold greater concentration was recently demonstrated to enhance procoagulant via enhanced activation of prothrombin in human plasma (see, Nielsen V G (2021) J Thromb Thrombolysis, doi: 10.1007/s11239-020-02373-4).

Data are presented as mean±SD. Experimental conditions were represented by n=6 replicates per condition as this provides a statistical power >0.8 with P<0.05 using this thrombelastographic methodology (see, Nielsen V G (2019) J Thromb Thrombolysis 48, 256-262; Nielsen V G, Frank N, Matika R W (2018) Biometals 31, 51-59; Nielsen V G, Frank N, Turchioe B J (2019) Blood Coagul Fibrinolysis 30, 379-384; Nielsen V G (2021) J Thromb Thrombolysis, doi: 10.1007/s11239-020-02373-4). A commercially available statistical program was used for one-way analyses of variance (ANOVA) comparisons between conditions, followed by Holm-Sidak post hoc analysis (SigmaPlot 14, Systat Software, Inc., San Jose, CA, USA). P<0.05 was considered significant.

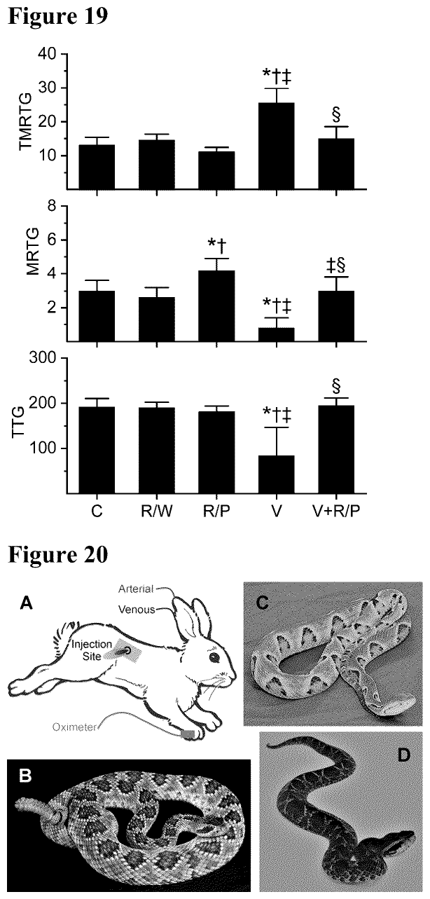

The results of these experiments are displayed in FIG. 19. With regard to RuCl₃ effects on plasma in the absence of venom, it appeared that a mild to moderate procoagulant effect was observed in RuCl₃ dissolved in PBS, is a significant increase in MRTG values (37-61%) noted compared to RuCl₃ dissolved in water or plasma without RuCl₃ addition. There were no significant differences between the solvent used for RuCl₃ or plasma without RuCl₃ addition when considering changes in plasmatic TMRTG or TTG values. Similar to the previous report (see, Nielsen V G (2019) J Thromb Thrombolysis 48, 256-262), venom significantly prolonged TMRTG values (195%), decreased MRTG values (63%) and decreased TTG values (56%) compared to control condition values. Lastly, exposing venom to RuCl₃ dissolved in PBS resulted in a significant decrease in TMRTG values (47%), increase in MRTG values (375%) and increase in TTG values (231%) in plasma compared to RuCl₃ naïve venom condition values. Lastly, there were no significant differences in TMRTG, MRTG or TTG values between the control condition and venom exposed to RuCl₃ dissolved in PBS condition.

The results described in Example V demonstrated that RuCl₃ dissolved in PBS inhibited the neurotoxic, anticoagulant PLA₂ activity contained within Mojave rattlesnake venom type A to the same extent as that observed with CORM-2 at an equimolar concentration (see, Nielsen V G (2019) J Thromb Thrombolysis 48, 256-262). Even with the caveat that RuCl₃ under these circumstances still exerts a measurable procoagulant effect in the absence of venom, the degree by which the anticoagulant activity is inhibited by RuCl₃ far overshadows kinetically the effect seen by RuCl₃ in the absence of venom. In conclusion, the data presented with a Ru-based ion rather than Ru-based radical inhibiting anticoagulant PLA₂ activity significantly contributes to the growing body of knowledge that Ru containing compounds may interact with key amino acid residues critical to the function of several classes of snake venom enzyme and potentially irreversibly inhibiting them.

### Example VI

Venomous snake bite is responsible for severe morbidity and mortality worldwide, affecting millions of people yearly [Warrell, D. A. Snake bite. Lancet 2010, 375, 77-88]. While financially prosperous countries may have antivenoms available, treatment is very expensive (tens of thousands of USD), injuries may still be severe/permanent, and there are not antivenoms available for all venoms. These symptoms are caused by the myriad of enzymes, peptides and other small molecular weight compounds that are contained in the snake venom, with metalloproteinases (SVMP), serine proteases (SVSP) and phospholipase A₂ (PLA₂) responsible for a great deal of loss of coagulation function, tissue damage and paralysis [Kang, T. S.; Georgieva, D.; Genov, N.; Murakami, M. T.; Sinha, M.; Kumar, R. P.; Kaur, P.; Kumar, S.; Dey, S.; Sharma, S.; Vrielink, A.; Betzel, C.; Takeda, S.; Ami, R. K.; Singh, T. P.; Kim, R. M. Enzymatic toxins from snake venom: structural characterization and mechanism of catalysis. FEBS J 2011, 278, 4544-4576]. In order to design and test novel antivenom strategies, a reliable preclinical animal model is needed that closely resembles humans, especially regarding the coagulation system. Rabbits can serve in this regard, as their thrombelastographic profile and scanning electron micrograph images of whole blood and plasmatic thrombus formation are very similar to that of humans [Nielsen, V. G.; Pretorius, E. Carbon monoxide: Anticoagulant or procoagulant? Thromb Res 2014, 133, 315-321]. In initial studies with intravenous injection of the venom of Crotalus atrox (Western diamondback rattlesnake), degradation of coagulation in both whole blood and platelet-inhibited whole blood were demonstrated in a sedated rabbit with thrombelastography [Nielsen, V. G.; Sánchez, E. E.; Redford, D. T. Characterization of the Rabbit as an In Vitro and In Vivo Model to Assess the Effects of Fibrinogenolytic Activity of Snake Venom on Coagulation. Basic Clin Pharmacol Toxicol 2018, 122, 157-164]. Further, in vitro exposure of this venom to tricarbonyldichlororuthenium (II) dimer (carbon monoxide releasing molecule 2, CORM-2) attenuated the anticoagulant effects of the intravenously administered venom in this rabbit model [Nielsen, V. G. Crotalus atrox Venom Exposed to Carbon Monoxide Has Decreased Fibrinogenolytic Activity In Vivo in Rabbits. Basic Clin Pharmacol Toxicol 2018, 122, 82-86]. Thus, the effects of systemic envenomation in a minimally sedated rabbit model, and attenuation of consequent venom mediated coagulopathy by an inorganic antivenom, were first documented [Nielsen, V. G. Crotalus atrox Venom Exposed to Carbon Monoxide Has Decreased Fibrinogenolytic Activity In Vivo in Rabbits. Basic Clin Pharmacol Toxicol 2018, 122, 82-86].

However, and thankfully, most venomous snake bites do not involve immediate release of most of the venom into the venous system, but instead is primarily released via the lymphatic system [van Helden, D. F.; Thomas, P. A.; Dosen, P. J.; Imtiaz, M. S.; Laver, D. R.; Isbister, G. K. Pharmacological approaches that slow lymphatic flow as a snakebite first aid. PLoS Negl Trop Dis 2014, 8, e2722; Paniagua, D.; Vergara, I.; Romin, R.; Romero, C.; Benard-Valle, M.; Calderón, A.; Jimenez, L.; Bernas, M. J.; Witte, M. H.; Boyer, L. V.; Alagón, A. Antivenom effect on lymphatic absorption and pharmacokinetics of coral snake venom using a large animal model. Clin Toxicol (Phila) 2019, 57, 727-734; van Helden, D. F.; Dosen, P. J.; O'Leary, M. A.; Isbister, G. K. Two pathways for venom toxin entry consequent to injection of an Australian elapid snake venom. Sci Rep 2019, 9, 8595], with some adsorption by veins [van Helden, D. F.; Dosen, P. J.; O'Leary, M. A.; Isbister, G. K. Two pathways for venom toxin entry consequent to injection of an Australian elapid snake venom. Sci Rep 2019, 9, 8595]. Thus, after physical disruption (via fangs) and venom enzymatic action, venom transport to the systemic circulation would be expected to depend on regional lymphatic flow [van Helden, D. F.; Thomas, P. A.; Dosen, P. J.; Imtiaz, M. S.; Laver, D. R.; Isbister, G. K. Pharmacological approaches that slow lymphatic flow as a snakebite first aid. PLoS Negl Trop Dis 2014, 8, e2722; Paniagua, D.; Vergara, I.; Román, R.; Romero, C.; Benard-Valle, M.; Calderón, A.; Jimenez, L.; Bemas, M. J.; Witte, M. H.; Boyer, L. V.; Alagón, A. Antivenom effect on lymphatic absorption and pharmacokinetics of coral snake venom using a large animal model. Clin Toxicol (Phila) 2019, 57, 727-734]. As lymphatic flow is decreased by inhalational general anesthetics and likely decreased some by the immobility of intravenous anesthesia [Bachmann, S. B.; Proulx, S. T.; He, Y.; Ries, M.; Detmar, M. Differential effects of anaesthesia on the contractility of lymphatic vessels in vivo. J Physiol 2019, 597, 2841-2852], a minimally sedated and humane animal model that involved no physical restraints following envenomation would more closely approximate the clinical situation of human envenomation. Other critical factors that may affect the toxicodynamic effects on coagulation following envenomation include consistent venom injection into the same region and depth in the rabbit, as it has very consistent regional lymphatic system anatomy [Soto-Miranda, M. A.; Suami, H.; Chang, D. W. Mapping superficial lymphatic territories in the rabbit. Anat Rec (Hoboken) 2013, 296, 965-970]; further, some snake venoms, such as that derived from the South American viper Bothrops asper (the Fer-de-lance), can significantly decrease lymphatic flow via the action of a myotoxic phospholipase A₂ (PLA₂) [Mora, J.; Mora, R.; Lomonte, B.; Gutiérrez, J. M. Effects of Bothrops asper snake venom on lymphatic vessels: insights into a hidden aspect of envenomation. PLoS Negl Trop Dis 2008, 2, e318]. As a final issue, in the two studies this author found that used subcutaneous envenomation in rabbits to assess changes in coagulation with standard laboratory methods [Fahmi, L.; Makran, B.; Boussadda, L.; Lkhider, M.; Ghalim, N. Haemostasis disorders caused by envenomation by Cerastes cerastes and Macrovipera mauritanica vipers. Toxicon 2016, 116, 43-48; Krishnan, L. K.; Saroja, J. B.; Rajalingam, M.; John, V.; Valappil, M. P.; Sreelatha, H. V. Rabbit snake-bite model to assess safety and efficacy of anti viper chicken antibodies (IgY). American Journal of Clinical and Experimental Medicine 2015, 3, 32-38], despite being approved by their institutions, animals were subjected to multiple envenomations without sedation or analgesia in both, suffered up to a 50% mortality with severe tissue damage noted in one, and lastly did not record any animal vital signs in both. This manner of investigation would not be acceptable in this institution; thus, the first goal of this study was to establish a minimally sedated, monitored, rabbit model of subcutaneous envenomation as displayed in FIG. 20, panel A.

The selection of venoms to be tested in the present study was carefully conducted based on proteome, previous in vitro characterization of the coagulopathy observed via thrombelastography, and the likelihood that the hemotoxic enzymes of the venom would be inhibited by ruthenium based, inorganic antivenoms [Nielsen, V. G. Carbon monoxide inhibits the anticoagulant activity of Mojave rattlesnake venoms type A and B. J Thromb Thrombolysis 2019, 48, 256-262; Nielsen, V. G. Modulation of Diverse Procoagulant Venom Activities by Combinations of Platinoid Compounds. Int J Mol Sci 2021, 22, 4612]. The first venom, derived from Crotalus scutulatus scutulatus (type B) (Mojave Rattlesnake, FIG. 20, panel B), is hemotoxic and primarily fibrinogenolytic; a concentration of 250 ng/ml of venom diminishes the velocity of thrombus formation and final clot strength in human plasma by 75% [Nielsen, V. G. Carbon monoxide inhibits the anticoagulant activity of Mojave rattlesnake venoms type A and B. J Thromb Thrombolysis 2019, 48, 256-262]. This venom is Janus-like in its action on human platelets, as a concentration of 300 μg/ml decreases epinephrine induced platelet aggregation by 25% [Carstairs, S. D.; Kreshak, A. A.; Tanen, D. A. Crotaline Fab antivenom reverses platelet dysfunction induced by Crotalus scutulatus venom: an in vitro study. Acad Emerg Med 2013, 20, 522-525], while a concentration of 40 mg/ml causes complete platelet aggregation [Corrigan, J. J. Jr.; Jeter, M. A. Mojave rattlesnake (Crotalus scutulatus scutulatus) venom: in vitro effect on platelets, fibrinolysis, and fibrinogen clotting. Vet Hum Toxicol 1990, 32, 439-441]. Thus, given the multiple orders of magnitude of effect by concentration [Nielsen, V. G. Carbon monoxide inhibits the anticoagulant activity of Mojave rattlesnake venoms type A and B. J Thromb Thrombolysis 2019, 48, 256-262; Carstairs, S. D.; Kreshak, A. A.; Tanen, D. A. Crotaline Fab antivenom reverses platelet dysfunction induced by Crotalus scutulatus venom: an in vitro study. Acad Emerg Med 2013, 20, 522-525; Corrigan, J. J. Jr.; Jeter, M. A. Mojave rattlesnake (Crotalus scutulatus scutulatus) venom: in vitro effect on platelets, fibrinolysis, and fibrinogen clotting. Vet Hum Toxicol 1990, 32, 439-441], it would be predicted that the primary effect of C. scutulatus scutulatus would be a decrease in plasmatic coagulation in vivo. The second venom, derived from Bothrops moojeni (Brazilian lancehead, FIG. 20, panel C), is hemotoxic and possesses a prothrombotic proteome that would be expected to activate and consume platelets and plasmatic coagulation proteins as part of the coagulopathy observed in the envenomed [Aguiar, W. D. S.; Galizio, N. D. C.; Serino-Silva, C.; Sant'Anna, S. S.; Grego, K. F.; Tashima, A. K.; Nishiduka, E. S.; Morais-Zani, K.; Tanaka-Azevedo, A. M. Comparative compositional and functional analyses of Bothrops moojeni specimens reveal several individual variations. PLoS ONE 2019, 14, e0222206]. Lastly, the venom obtained from Calloselasma rhodostoma (Malayan pit viper, FIG. 20, panel D), is hemotoxic and contains a variety of procoagulant serine proteases, one in particular (ancrod) that was used medicinally for defibrinogenation, although consumption of both platelets and plasmatic coagulation proteins are observed [Tang, E. L.; Tan, C. H.; Fung, S. Y.; Tan, N. H. Venomics of Calloselasma rhodostoma, the Malayan pit viper: A complex toxin arsenal unraveled. J Proteom 2016, 148, 44-56]. Thus, the first goal of the present study was to create a novel rabbit model to characterize toxicodynamic profiles of the coagulopathies caused by these diverse venoms. Lastly, the second goal was to administer ruthenium based antivenoms demonstrated to abrogate the anticoagulant [Nielsen, V. G. Carbon monoxide inhibits the anticoagulant activity of Mojave rattlesnake venoms type A and B. J Thromb Thrombolysis 2019, 48, 256-262] and procoagulant [Nielsen, V. G. Modulation of Diverse Procoagulant Venom Activities by Combinations of Platinoid Compounds. Int J Mol Sci 2021, 22, 4612] effects of these venoms in vitro into the venom injection site, with the goal of potentially diminishing venom mediated coagulopathy.

### Materials and Methods

### Chemicals and Venoms

Lyophilized venoms derived from C. scutulatus scutulatus (type B), B. moojeni and C. rhodostoma were provided by the National Natural Toxins Research Center (NNTRC) located at Texas A&M University-Kingsville, Kingsville, TX, U.S.A. The National Institutes of Health fund the NNTRC out of the Office of Research Infrastructure Programs. Venoms were dissolved into calcium-free phosphate buffered saline (PBS, Millipore Sigma, Saint Louis, MO, USA) to a final 30 mg/mL concentration, aliquoted, and maintained at −80° C. Dimethyl sulfoxide (DMSO), tricarbonyldichlororuthenium (II) dimer (CORM-2), and RuCl₃ were obtained from Millipore Sigma (Saint Louis, MO, USA). Tissue factor for activating coagulation was obtained in the form of Pacific Hemostasis™ Prothrombin Time Reagent, Thermo Fisher Scientific, Pittsburgh, PA, USA). Calcium chloride (200 mM) was obtained from Haemonetics Inc. (Braintree, MA, USA).

### Rabbit Model

Male New Zealand White rabbits (2-3 kg) were procured from Charles River Laboratories (San Diego, CA, USA) and housed within our animal facility and allowed food and water ad libitum for at least 1 week prior to experimentation. The Institutional Animal Care and Utilization Committee of the University of Arizona approved all procedures involving these rabbits. The protocol was conducted in accordance with all applicable federal and institutional policies, procedures, and regulations, including the PHS Policy on Humane Care and Use of Laboratory Animals, USDA regulations (9 CFR Parts 1, 2, 3), the Federal Animal Welfare Act (7 USC 2131 et. Seq.), the Guide for the Care and Use of Laboratory Animals, and all relevant institutional regulations and policies regarding animal care and use at the University of Arizona.

Rabbits were briefly restrained and had one ear closely clipped and cleaned with a 70% isopropyl alcohol pad. A 22 G plastic catheter was placed in the central ear artery and another similar catheter placed in the marginal ear vein; both catheters were connected to an end cap with a rubber diaphragm that allowed the withdrawal of blood samples and administration of medications. The animals were sedated intravenously with 1 mg/kg midazolam, with supplemental doses of 0.5-1 mg/kg provided during experimentation to maintain sedation. As displayed in FIG. 1, panel A, one toe of a forepaw was subsequently closely clipped, with a pulse oximeter probe placed to monitor heart rate (HR, beats/min) and % arterial oxygenation (SpO₂) with a CMS60D-VET SP02 Pulse Oximeter (CONTEC™, Qinhuangdao (Hebei), China). Heart rate (HR) and SpO₂ were recorded at baseline and every 15 min thereafter until the end of the experiment.

An approximately 5 cm by 5 cm area of skin over either flank of the rabbit, midway between the lumbar spine and mid abdomen, was closely shaved and cleaned with a 70% isopropyl alcohol pad. A 1 cm circle was drawn with a felt tip marker in the center of this area, which served as the injection site for venom and antivenom as appropriate. After obtaining baseline HR value, SpO₂ value, and the initial blood sample, venom was injected subcutaneously in the middle of the circle through a ⅝ inch long, 25 G needle. The initial dose of each venom was based on a value obtained from mice lethal dose 50% studies (LD₅₀), with the dose for rabbits beginning with approximately half of the LD₅₀ dose. Thus, the initial subcutaneous doses for each venom were as follows: C. scutulatus scutulatus (1.4 mg/kg) [Glenn, J. L.; Straight, R. Mojave rattlesnake Crotalus scutulatus scutulatus venom: variation in toxicity with geographical origin. Toxicon 1978, 16, 81-84], FIG. 20, panel B; B. moojeni (3.0 mg/kg) [Furtado, M. F.; Maruyama, M.; Kamiguti, A. S.; Antonio, L. C. Comparative study of nine Bothrops snake venoms from adult female snakes and their offspring. Toxicon 1991, 29, 219-226] FIG. 20, panel C, and C. rhodostoma (3.0 mg/kg) [Pommanee, P.; Sánchez, E. E.; López, G.; Petsom, A.; Khow, O.; Pakmanee, N.; Chanhome, L.; Sangvanich, P.; Pérez, J. C. Neutralization of lethality and proteolytic activities of Malayan pit viper (Calloselasma rhodostoma) venom with North American Virginia opossum (Didelphis virginiana) serum. Toxicon 2008, 52, 186-189], FIG. 20, panel D. The dose of venom was increased or decreased until a consistent pattern of coagulopaty was observed; thereafter, this dose was used to characterize the coagulopathy and to test the efficacy of antivenom as subsequently described. Blood samples were collected from this time point onward every hour for 3 hours. If the animal was to be administered antivenom, then this was performed 5 minutes after injection of the venom, again administered through a 25 G needle. Antivenom was composed of one of three doses and contents: 1) CORM-2 in PBS at a concentration of 10 mg/ml, administered at a dose of 1 ml/kg; and, 2) CORM-2 10 mg/ml in a PBS containing 500 μM RuCl₃ solution at a dose of 2 ml/kg. Antivenom solutions were made freshly just after the venom injection over a 3 minute period prior to administration. Ten minutes after the venom injection, a 1.5 cm by 1.5 cm piece of a 4% lidocaine analgesic patch (Lidocaine Pain Relief Patch, Walgreens, Walgreen Company, Deerfield, IL, USA) was placed over the injection site to minimize discomfort for the remainder of the experiment. Lastly, after the last blood sample and vital sign assessments were obtained, the rabbits were euthanized with intravenous administration of 1 ml of pentobarbital/phenytoin (390/50 mg/ml).

### Coagulation Monitoring.

Blood was collected prior to envenomation, designated the baseline sample, and then every hour after envenomation for three hours. Blood collected during the experiments involving Mojave rattlesnake venom was placed into a sodium citrate containing tube (2.7 ml blood; 9 parts blood to 1 part 0.105 M sodium citrate), with an aliquot removed for whole blood coagulation evaluation. The remaining blood was subjected to centrifugation at 3000×g for 15 minutes at room temperature, with plasma decanted and coagulation kinetics assessed as subsequently described. In experiments involving the Brazilian lancehead and Malayan pit viper, whole blood samples (1 ml) were quickly collected, with aliquots placed immediately into thrombelastographic (TEG) cups for analysis as subsequently presented. The rationale for this approach with the latter two venoms was that it was noted that blood could clot within the citrate containing tubes within just a few during preliminary experiments, indicative of venom enzymatic activity that was calcium independent that would confound the coagulation assessment. Thus, to minimize this in vitro artifact, blood was rapidly placed into thrombelastographic cups with activation by tissue factor.

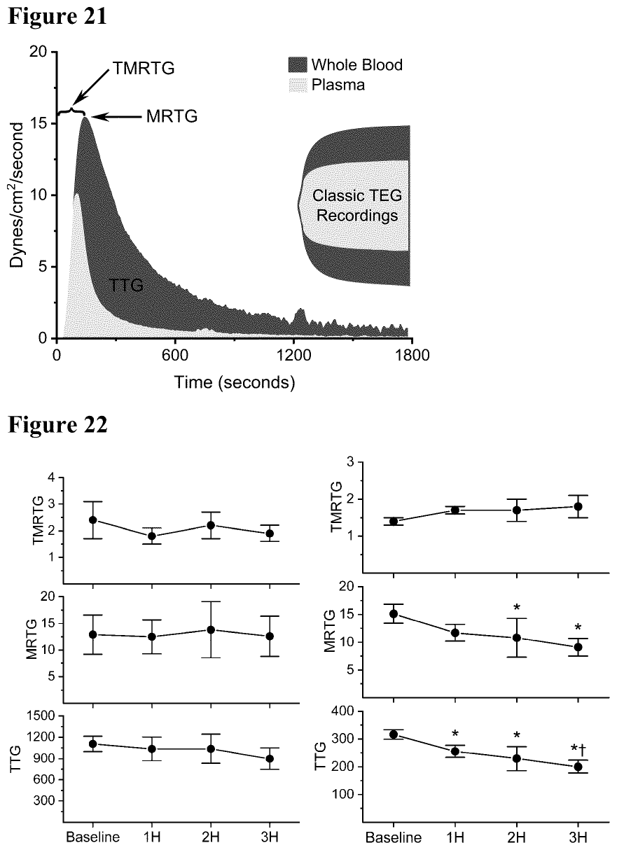

All sample mixtures were placed in a disposable cup in a computer-controlled Thrombelastograph® haemostasis system (Model 5000; Haemonetics Inc., Braintree, MA, USA) at 39° C., the normal temperature of the NZW rabbit. The mixture used in the series of experiments involving Mojave rattlesnake venom was composed of 330 μl of whole blood or plasma, 10 μl of tissue factor (0.10% final concentration of Pacific Hemostasis™ Prothrombin Time Reagent, Thermo Fisher Scientific, Pittsburgh, PA, USA) and 20 μl of 200 mM CaCl₂) (Haemonetics Inc.). In the series of experiments involving the Brazilian lancehead and Malayan pit viper, the sample mixture was 350 μl of whole blood and 10 μl of tissue factor. After mixing the samples by raising and lowering the cup to the level of the thrombelastographic disposable pin five times, the following elastic modulus-based parameters were determined: time to maximum rate of thrombus generation (TMRTG), this is the time interval (minutes or seconds) observed prior to maximum speed of clot growth; maximum rate of thrombus generation (MRTG), this is the maximum velocity of clot growth observed (dynes/cm²/second); and total thrombus generation (TTG, dynes/cm²), the final viscoelastic resistance observed after clot formation. Data were collected for 30 minutes. These variables as measured from a whole blood and plasma sample obtained from a rabbit under baseline conditions are displayed in FIG. 21 with corresponding coagulation velocity curves. Also, the corresponding whole blood and plasma TEG data that are observed in the computer screen as thrombi are formed in the device are presented in the right side of FIG. 21. Lastly, in the series of experiments involving C. scutulatus scutulatus envenomation, the contribution of platelets to overall clot strength was deterimined with the following equation: Platelet mediated strength (%)=((TTG of whole blood−TTG of plasma)/TTG of whole blood)×100%.

### Statistical Analyses.

Data are presented as mean±SD. All experimental groups were represented by n=5-7 different individuals, as this provided a statistical power >0.8 with p<0.05 using this methodology to assess differences in thrombelastographic parameters within and between groups. A commercially available statistical program was used for one-way or two-way, repeated measures analyses of variance (ANOVA) as appropriate to the dataset, followed by Holm-Sidak post hoc analyses (SigmaStat 3.1; Systat Software, Inc., San Jose, CA, USA). Graphics were generated with commercially available programs; Origen 2023, OrigenLab Corporation, Northampton, MA, USA; and, CorelDRAW Home & Student, Alludo, Ottawa, ON, Canada). p<0.05 was considered significant.

### Results

Effects of C. scutulatus scutulatus Envenomation on Whole Blood and Plasmatic Coagulation and Efficacy of Antivenom.

Rabbits were initially dosed with 1.4 mg/kg [Glenn, J. L.; Straight, R. Mojave rattlesnake Crotalus scutulatus scutulatus venom: variation in toxicity with geographical origin. Toxicon 1978, 16, 81-84], but no effect was seen in whole blood or plasmatic coagulation. Subsequent doses of 2.8, 5.6, and 11.2 mg/kg were asssessed, with the 11.2 mg/kg dose finally resulting in loss of plasmatic coagulation. A total of five rabbits were analyzed with this dose. A general observation was that the rabbits exhibited no signs of distress or behaviors of pain at the venom injection site. Further, there was no significant change in either heart rate or SpO₂ throughout experimentation. The results of the vital signs recorded are displayed in table 3.

TABLE 3
HR and SpO₂ during experimentation with C. scutulatus scutulatus venom.

| Time | BSL | 15 | 30 | 45 | 60 | 75 | 90 | 105 | 120 | 135 | 150 | 165 | 180 |
|---|---|---|---|---|---|---|---|---|---|---|---|---|---|
| HR | 192 ± 39 | 185 ± 33 | 183 ± 40 | 185 ± 33 | 176 ± 37 | 171 ± 12 | 168 ± 14 | 169 ± 8 | 188 ± 35 | 177 ± 19 | 172 ± 17 | 178 ± 13 | 168 ± 22 |
| SpO₂ | 98 ± 1 | 98 ± 1 | 98 ± 1 | 98 ± 2 | 96 ± 3 | 97 ± 2 | 98 ± 1 | 98 ± 1 | 98 ± 1 | 98 ± 1 | 97 ± 2 | 95 ± 2 | 98 ± 2 |

Time: BSL = baseline; the remaining numbers are time in minutes after venom injection. Data presented as mean ± SD.

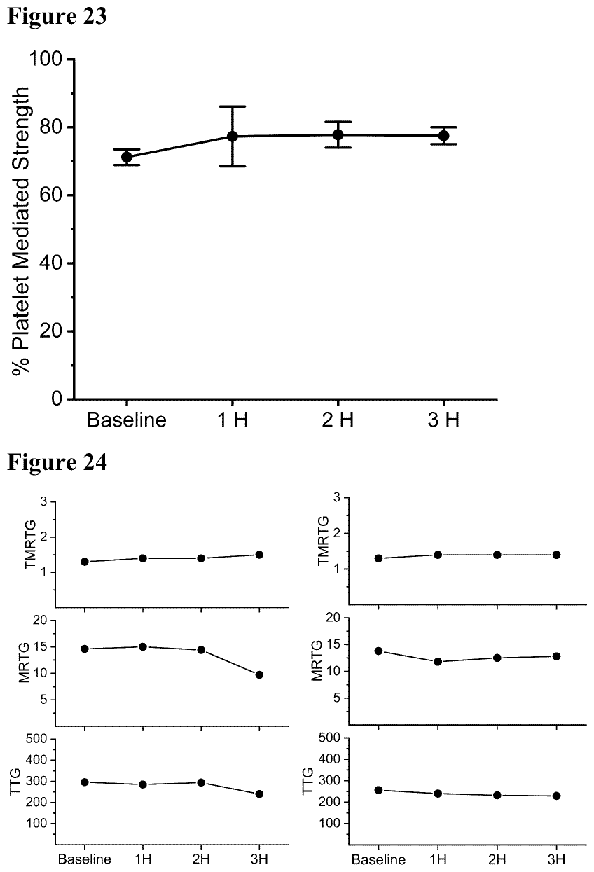

With regard to coagulation, this dose of C. scutulatus scutulatus venom had no significant effect on whole blood coagulation as presented in FIG. 22, left panel. In contrast, plasmatic coagulation had a significant decrease in MRTG values two and three hours after envenomation compared to baseline values as noted in FIG. 22, right panel. Further, a decrease in TTG values throughout experimentation after enveomation was also observed. Also of interest, there was no change in the contribution of platelet mediated clot strength as displayed in FIG. 23. These data are characteristic of the effects of a primarily fibrinogenolytic venom with minimal effects on platelets [Nielsen, V. G. Carbon monoxide inhibits the anticoagulant activity of Mojave rattlesnake venoms type A and B. J Thromb Thrombolysis 2019, 48, 256-262; Carstairs, S. D.; Kreshak, A. A.; Tanen, D. A. Crotaline Fab antivenom reverses platelet dysfunction induced by Crotalus scutulatus venom: an in vitro study. Acad Emerg Med 2013, 20, 522-525; Corrigan, J. J. Jr.; Jeter, M. A. Mojave rattlesnake (Crotalus scutulatus scutulatus) venom: in vitro effect on platelets, fibrinolysis, and fibrinogen clotting. Vet Hum Toxicol 1990, 32, 439-441].

The next phase of experimentation was to determine a dose of ruthenium-based antivenom to inject into the venom injection site. Given that the amount of venom required to cause this significant decrease in plasmatic coagulation was 8-fold greater than originally anticipated, it was decided to inject a large dose of CORM-2, 10 mg/kg delivered as 10 mg/ml PBS. This single agent very effectively abrogated the anticoagulant effects of C. scutulatus scutulatus venom in vitro [Nielsen, V. G. Carbon monoxide inhibits the anticoagulant activity of Mojave rattlesnake venoms type A and B. J Thromb Thrombolysis 2019, 48, 256-262].

As this venom only affected plasmatic coagulation, only the results from data collected from plasma samples are displayed in FIG. 24. As can be observed in the left panel of FIG. 24, the first experiment involving the injection of 10 mg/kg CORM-2 appeared to preserve the velocity of clot formation and strength until the third hour after venom injection. Subsequently, in the next experiment, 20 mg/kg of CORM-2 was administered, and it seemed that both the velocity of clot formation were minimally affected for the three hours after venom injection.

Encouraged by these results, it was planned to proceed with a new series of experiments with rabbits administered the previously mentioned dose of venom without or with antivenom administered. However, the preliminary studies used nearly all the venom purchaced as the dose required was 8-fold greater than anticipated. The author was disappointed to learn that the source of the venom, the NNTRC in Texas, did not have sufficient venom to provide for these anticipated studies and was without a snake to collect more venom. Other sources of venom could not be identified by the NNTRC, so the author is unable to provide further information concerning C. scutulatus scutulatus, type B, envenomation with this novel rabbit model at this time. Nevertheless, the author was able to learn from the aforementioned data and experiences to subsequently proceed with investigations with the subsequently presented venoms, which are in plentiful supply.

Effects of B. moojeni Envenomation on Whole Blood Coagulation and Efficacy of Antivenom.

The first rabbit of this series was dosed with 3.0 mg/kg [Furtado, M. F.; Maruyama, M.; Kamiguti, A. S.; Antonio, L. C. Comparative study of nine Bothrops snake venoms from adult female snakes and their offspring. Toxicon 1991, 29, 219-226]. While the first blood sample collected before envenomation was separated into whole blood and plasma without any problem, the sample collected one hour after envenomation was observed to have the citrate anticoagulated whole blood sample contain some clotted material. Further, the plasma retrieved from the cetrifuged sample was also solidified despite anticoagulation. It was apparent that this venom contained calcium-independent, procoagulant enzymes that were rapidly acting at this dose. To deal this this issue, it was decided to rapidly collect whole blood and place it into the thrombelastographic cups with immediate activation with tissue factor as noted in the previously described methods. The time of collection to the time of onset of analysis was routinely under one minute, which should have outcompeted the procoagulant venom enzymes and allow an assessment of tissue factor initiated coagulation with the remaining components of the rabbit's blood.

The second and third rabbits were administered 1.5 mg/kg of B. moojeni venom, and they displayed a consistent and remarkable pattern of coagulopathy over the three hours of observation after envenomation. Thus, this was the dose subsequently used for the remainder of experiments with this venom.

With regard to the composition and dose of antivenom chosen to be administered after envenomation, a combination of CORM-2 and RuCl₃ was demostrated to more effectively inhibit the procoagulant activity of the venom compared to either ruthenium containing compound in isolation [Nielsen, V. G. Modulation of Diverse Procoagulant Venom Activities by Combinations of Platinoid Compounds. Int J Mol Sci 2021, 22, 4612]. Further, as 20 mg/kg CORM-2 appeared to be potentially more effective than 10 mg/kg, the larger dose was selected. Thus, the dose administered was CORM-2 10 mg/ml in a PBS containing 500 μM RuCl₃ solution at a dose of 2 ml/kg.

With regard to the clinical state of the rabbbits, as with the previous series of experiments, the rabbits exhibited no signs of distress or behaviors of pain at the venom injection site, a pattern that persisted either without or with the addition of antivenom injection. Further, there was no significant change in either heart rate or SpO₂ throughout experimentation. The results of the vital signs recorded are displayed in tables 4 and 5.

TABLE 4
HR during experimentation with B. moojeni venom.

| Time | BSL | 15 | 30 | 45 | 60 | 75 | 90 | 105 | 120 | 135 | 150 | 165 | 180 |
|---|---|---|---|---|---|---|---|---|---|---|---|---|---|
| V | 218 ± 21 | 222 ± 18 | 208 ± 36 | 194 ± 35 | 214 ± 29 | 204 ± 34 | 196 ± 39 | 209 ± 29 | 214 ± 25 | 212 ± 25 | 218 ± 22 | 216 ± 38 | 219 ± 22 |
| A + V | 194 ± 41 | 222 ± 32 | 242 ± 15 | 203 ± 43 | 218 ± 26 | 226 ± 29 | 228 ± 28 | 217 ± 33 | 213 ± 29 | 221 ± 20 | 220 ± 14 | 221 ± 19 | 228 ± 18 |

Time: As in table 3. V = venom injection; A + V = venom injection followed by antivenom injection. Data presented as mean ± SD.

TABLE 5
SpO₂ during experimentation with B. moojeni venom.

| Time | BSL | 15 | 30 | 45 | 60 | 75 | 90 | 105 | 120 | 135 | 150 | 165 | 180 |
|---|---|---|---|---|---|---|---|---|---|---|---|---|---|
| V | 98 ± 2 | 97 ± 1 | 96 ± 3 | 98 ± 2 | 96 ± 3 | 96 ± 3 | 98 ± 2 | 98 ± 1 | 99 ± 1 | 98 ± 2 | 98 ± 2 | 98 ± 2 | 98 ± 1 |
| A + V | 98 ± 2 | 96 ± 2 | 96 ± 3 | 97 ± 2 | 98 ± 1 | 98 ± 2 | 95 ± 3 | 97 ± 2 | 97 ± 2 | 97 ± 2 | 98 ± 2 | 97 ± 2 | 97 ± 2 |

Time: As in table 3. V = venom injection; A + V = venom injection followed by antivenom injection. Data presented as mean ± SD.

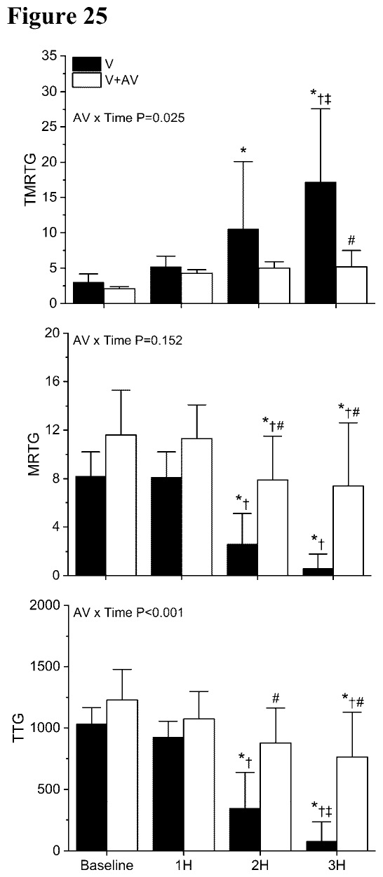

As displayed in FIG. 25, B. moojeni venom had little effect on coagulation one hour after injection. However, by two hours, envenomed rabbits demonstrated a significant loss of coagulation function as evidenced as an increase in TMRTG and decrease in both MRTG and TTG values. This loss of coagulation function became far more severe by the third hour in envenomed rabbits. In sharp contrast, rabbits injected with antivenom did not have any significant change in TMRTG values over the three hours post venom injection. Further, while there was a significant decrease in MRTG and TTG values during the second and third hour following venom injection, rabbits administered antivenom had significantly less deterioration of coagulation function compared to animals not administered antivenom. Lastly, the interaction of time and antivenom administration was significant in the cases of TMRTG and TTG values as seen in the top and bottom panels of FIG. 25.

Effects of C. rhodostoma Envenomation on Whole Blood Coagulation and Efficacy of Antivenom

The first rabbit of this series was dosed with 3.0 mg/kg [Pommanee, P.; Sánchez, E. E.; López, G.; Petsom, A.; Khow, O.; Pakmanee, N.; Chanhome, L.; Sangvanich, P.; Pérez, J. C. Neutralization of lethality and proteolytic activities of Malayan pit viper (Calloselasma rhodostoma) venom with North American Virginia opossum (Didelphis virginiana) serum. Toxicon 2008, 52, 186-189]. The pattern of coagulopathy with this rabbit and a second rabbit administered 3.0 mg/kg appeared somewhat similar to that observed with B. moojeni with 1.5 mg/kg. Subsequently, this was the dose of venom chosen for the remainder of this series of experiments with C. rhodostoma venom. The composition and dose of antivenom injected was the same as that used with B. moojeni, as the procoagulant activity of this venom was also more effectively inhibited by the two ruthenium containing compounds in combination compared to either in isolation [Nielsen, V. G. Modulation of Diverse Procoagulant Venom Activities by Combinations of Platinoid Compounds. Int J Mol Sci 2021, 22, 4612].

With regard to the clinical state of the rabbbits, as with the previous two series of experiments, the rabbits exhibited no signs of distress or behaviors of pain at the venom injection site or after of antivenom injection. With the exception of the baseline measurement of heart rate, there was no significant change in either heart rate or SpO₂ throughout experimentation within or between the two groups. The results of the vital signs recorded are displayed in tables 6 and 7.

TABLE 6
HR during experimentation with C. rhodostoma venom.

| Time | BSL | 15 | 30 | 45 | 60 | 75 | 90 | 105 | 120 | 135 | 150 | 165 | 180 |
|---|---|---|---|---|---|---|---|---|---|---|---|---|---|
| V | 214 ± 20 | 224 ± 28 | 220 ± 32 | 216 ± 19 | 232 ± 21 | 231 ± 28 | 231 ± 15 | 236 ± 13 | 223 ± 22 | 227 ± 19 | 232 ± 23 | 242 ± 22 | 239 ± 19 |
| A ± V | 239 ± 2* | 246 ± 6 | 237 ± 11 | 226 ± 16 | 216 ± 14 | 219 ± 9 | 218 ± 13 | 217 ± 9 | 218 ± 14 | 220 ± 13 | 219 ± 7 | 224 ± 9 | 223 ± 15 |

Time: As in table 3. V = venom injection; A + V = venom injection followed by antivenom injection. Data presented as mean ± SD.
*P < 0.05 vs. V.

TABLE 7
SpO₂ during experimentation with C. rhodostoma venom.

| Time | BSL | 15 | 30 | 45 | 60 | 75 | 90 | 105 | 120 | 135 | 150 | 165 | 180 |
|---|---|---|---|---|---|---|---|---|---|---|---|---|---|
| V | 97 ± 2 | 98 ± 2 | 98 ± 1 | 98 ± 1 | 97 ± 2 | 97 ± 2 | 98 ± 2 | 98 ± 2 | 98 ± 1 | 98 ± 2 | 98 ± 1 | 98 ± 1 | 99 ± 1 |
| A + V | 99 ± 1 | 97 ± 2 | 96 ± 2 | 98 ± 1 | 98 ± 1 | 99 ± 1 | 99 ± 1 | 98 ± 1 | 98 ± 1 | 98 ± 2 | 99 ± 1 | 98 ± 2 | 99 ± 1 |

Time: As in table 3. V = venom injection; A + V = venom injection followed by antivenom injection. Data presented as mean ± SD.
*P < 0.05 vs. V.

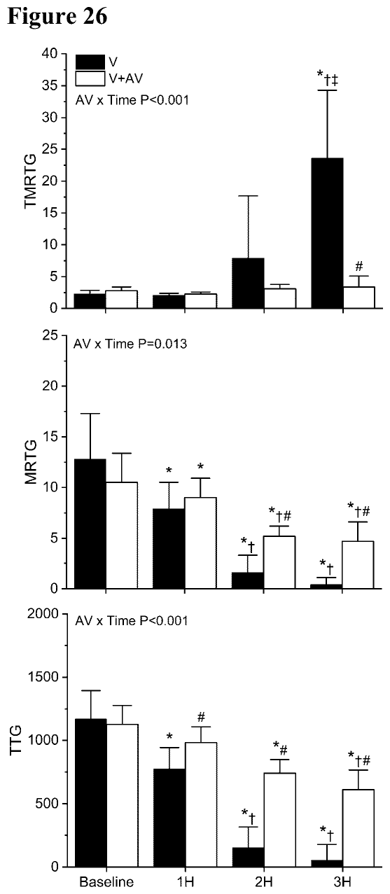

As displayed in FIG. 26, C. rhodostoma venom had degraded coagulation throughtout the observation period. A significant decrease in MRTG and TTG values were observed compared to baseline values at all three hours in rabbits injected with venom alone. A significant increase in TMRTG values was only observed at the three hour time point in animals administered venom without antivenom administration. By the third hour, coagulation function was markedly diminished in this group, very similar in magnitude to the decrease seen with B. moojeni envenomation displayed in FIG. 25. med rabbits. In sharp contrast, rabbits injected with antivenom did not have any significant change in TMRTG values over the three hours post venom injection, and TMRTG was significanly smaller in this group compared to the animals injected with venom alone. Further, while there was a significant decrease in MRTG and TTG values throughout the observation period following venom injection, rabbits administered antivenom had significantly less deterioration of coagulation function compared to animals not administered antivenom. Lastly, the interaction of time and antivenom administration was significant in the cases of TMRTG, MRTG and TTG values as seen in the panels of FIG. 26.

### Discussion

The present study successfully achieved its stated goals regarding the toxicodynamic characterization of diverse venoms with this minimally sedated rabbit model and assessment of the efficacy of ruthenium based antivenoms. Both of these goals will be subsequently discussed in detail.

The toxicodynamic characterizations of the three venoms chosen were remarkable. First, in the case of C. scutulatus scutulatus envenomation, a remarkable amount of venom (11.2 mg/kg) was needed to cause a significant decrease in plasmatic coagulation compared to the small concentration (250 ng/ml) required to compromise human plasmatic coagulation [Nielsen, V. G. Carbon monoxide inhibits the anticoagulant activity of Mojave rattlesnake venoms type A and B. J Thromb Thrombolysis 2019, 48, 256-262]. Possible mechanisms by which such large doses of venom were needed to cause changes in plasmatic coagulation include poor enzymatically mediated egress into the lymphatic system (given the minimal vascular trauma caused by a 25 G needle) or perhaps remarkable redistribution and elimination from the circulation of the animal. The relative plateau in compromised plasmatic coagulation after injection of C. scutulatus scutulatus venom supports the concept of redistribution of venom enzymes from target molecules (e.g., fibrinogen), a phenomenon observed when a bolus of Crotalus atrox (Western diamondback rattlesnake) venom is injected intravenously into the rabbit [Nielsen, V. G.; Sánchez, E. E.; Redford, D. T. Characterization of the Rabbit as an In Vitro and In Vivo Model to Assess the Effects of Fibrinogenolytic Activity of Snake Venom on Coagulation. Basic Clin Pharmacol Toxicol 2018, 122, 157-164; Nielsen, V. G. Crotalus atrox Venom Exposed to Carbon Monoxide Has Decreased Fibrinogenolytic Activity In Vivo in Rabbits. Basic Clin Pharmacol Toxicol 2018, 122, 82-86]. Lastly, the use of whole blood and plasma samples allowed the demonstration that this venom primarily affects plasmatic coagulation, without significant changes in platelet mediated coagulation noted. Regarding the second venom investigated, B. moojeni envenomation demonstrated a one hour “pause” after injection, followed by a rapid degradation of whole blood coagulation over the subsequent two hours of observation. This is consistent with the concept that this venom interfered with lymphatic flow as observed with other Bothrops species [Mora, J.; Mora, R.; Lomonte, B.; Gutiérrez, J. M. Effects of Bothrops asper snake venom on lymphatic vessels: insights into a hidden aspect of envenomation. PLoS Negl Trop Dis 2008, 2, e318]. The pattern of rapid destruction of whole blood coagulation by B. moojeni venom is remarkably different than that of C. scutulatus scutulatus venom, and it is very likely that whatever rate redistribution or elimination of B. moojeni venom occurs in the rabbit is out competed by the rate of catalysis of the venom enzymes. Lastly, the toxicodynamic pattern of coagulopathy of C. rhodostoma venom was the most impressive as there was no “pause” in the onset of whole blood coagulopathy as seen with B. moojeni venom. C. rhodostoma venom at the dose administered relentlessly destroyed coagulation function over the three-hour observation period, demonstrating rapid entry into the lymphatic system and likely continuous consumption of both cellular and plasmatic elements of coagulation. In summary, this rabbit model allowed for the identification of the toxicodynamic “fingerprint” of these three venoms that are diverse in proteome and geographical origin.

Assessment of the efficacy of the ruthenium based antivenoms was largely successful, with the limitation that there was insufficient C. scutulatus scutulatus venom available to perform enough experiments to statistically compare groups administered venom only or venom followed by antivenom injection. However, preliminary experiments with this venom influenced the composition and dose of ruthenium compound containing antivenom tested to abrogate the coagulopathic effects of the other two venoms. Protection from prolongation of TMRTG values after injection of either B. moojeni or C. rhodostoma venom (FIGS. 25 and 26) indicates that the antivenom prevented a critical loss of procoagulants that would prevent the normal onset of coagulation—a key function of hemostasis. Antivenom administration also decreased the velocity of the loss of MRTG and TTG values after B. moojeni or C. rhodostoma envenomation, with values several fold greater at three hours post venom injection compared to animals without antivenom injection (FIGS. 25 and 26). These patterns of protection are likely secondary to irreversible inhibition of key venom enzymes, with degradation of systemic coagulation caused by venom that either was not inhibited secondary to not being exposed to the antivenom within the injection site or perhaps by the venom gaining access to the lymphatic during the five minutes prior to antivenom injection. Thus, these data support the concept that this novel site directed, ruthenium compound-based approach, attenuated venom mediated degradation of whole blood, systemic coagulation function.

While a small animal model was employed to conduct this investigation, the paradigm of antivenom treatment was not organism focused but instead “bite site” focused. Put another way, treatment consisted of neutralizing the venom injected with direct antivenom application, not providing a circulating antivenom moiety with a long circulating half-life to inactivate venom enzymes as they enter the bloodstream. Given the potential nonspecific binding of the ruthenium radical formed during release of carbon monoxide to other biomolecules in the subcutaneous space, a large dose of CORM-2 was justified, and has previously been well tolerated when injected intravenously into rabbits [Nielsen, V. G.; Chawla, N.; Mangla, D.; Gomes, S. B.; Arkebauer, M. R.; Wasko, K. A.; Sadacharam, K.; Vosseller, K. Carbon monoxide-releasing molecule-2 enhances coagulation in rabbit plasma and decreases bleeding time in clopidogrel/aspirin-treated rabbits. Blood Coagul Fibrinolysis 2011, 22, 756-759]. Another dose of CORM-2, 20 mg/kg, was justified if 10 mg/kg was unsuccessful in diminishing venom activity, as up to 30 mg/kg of CORM-2 in a murine model of acute kidney injury was well tolerated and protected against injury [Uddin, M. J.; Jeong, J.; Pak, E. S.; Ha, H. CO-Releasing Molecule-2 Prevents Acute Kidney Injury through Suppression of ROS-Fyn-ER Stress Signaling in Mouse Model. Oxid Med Cell Longev 2021, 2021, 9947772]. As has been demonstrated with several venoms and venom enzymes, it is the ruthenium radical of CORM-2 that presumably binds to key amino acid residues such as histidine to inhibit activity [Nielsen, V. G.; Wagner, M. T.; Frank, N. Mechanisms Responsible for the Anticoagulant Properties of Neurotoxic Dendroaspis Venoms: A Viscoelastic Analysis. Int J Mol Sci 2020, 21, 2082; Nielsen, V. G. The anticoagulant effect of Apis mellifera phospholipase A₂ is inhibited by CORM-2 via a carbon monoxide-independent mechanism. J Thromb Thrombolysis 2020, 49, 100-107; Nielsen, V. G. Ruthenium, Not Carbon Monoxide, Inhibits the Procoagulant Activity of Athens, Echis, and Pseudonaja Venoms. Int J Mol Sci 2020, 21, 2970; Nielsen, V. G. Ruthenium chloride inhibits the anticoagulant activity of the phospholipase A₂-dependent neurotoxin of Mojave rattlesnake Type A venom. J Thromb Thrombolysis 2021, 52, 1020-1022; Pe, T.; Khin Aung Cho, K. A. Amount of venom injected by Russell's viper (Vipera russelli). Toxicon 1986, 24, 730-733]. Unfortunately, biomolecules such as albumin that are in the interstitial space contain such amino acids, which is what necessitates the administration of larger volumes and concentrations of ruthenium based antivenoms to successfully neutralize antivenom activity despite nonspecific binding to other compounds. Also of interest, dosing of the antivenom would be based on the amount of venom injected during the snake bite in larger organisms such as domestic animals and humans, so it is anticipated that a fixed dose of ruthenium based antivenom would be required to neutralize a range of venom volumes. As an example, adult Vipera russelli have been documented to inject on average 63 mg and up to 147 mg of venom (after desiccation) per bite [Pe, T.; Khin Aung Cho, K. A. Amount of venom injected by Russell's viper (Vipera russelli). Toxicon 1986, 24, 730-733]; thus, it would need to be determined with the presented rabbit model or via clinical trials what fixed dosage of ruthenium based antivenom would abrogate this amount of venom. In summary, while administration of both venom and antivenom are administered based on kg of the rabbit, the rabbit is serving as a “bite site” to assess molecular interactions between venom and ruthenium based antivenom.

This investigation has some limitations. First, it could be argued that five minutes seems to be a brief period prior to administering site directed antivenom. While this is understandable, it was the consideration that the subcutaneous space of the rabbit is only a few millimeters thick and well perfused that was considered when choosing the interval to delay antivenom treatment. Arguably, some venom will immediately enter the circulation secondary to the trauma of injection, and more venom will likely enter the lymphatic system at some unknown rate. Thus, no matter how effective the site directed antivenom may be, venom that has left the bite site will wreak havoc on the target cells and molecules in the circulation. This may be the scenario observed in the cases of envenomation by B. moojeni and C. rhodostoma when assessing changes in whole blood coagulation without or with antivenom administration in FIGS. 25 and 26. Given that the rabbit model is an artificial construct to assess toxicodynamic change in coagulation and antivenom efficacy, these results are somewhat expected. Other limitations of this investigation include not assessing multiple doses of all venoms used and a variety of concentrations and compositions of ruthenium based antivenom. Given that this work is a “proof-of-concept” work wherein the goals were limited and needless loss of animal life should be avoided, it is held that further experimentation is warranted, but with other venoms (e.g., Crotalus atrox, etc.) and antivenom doses in future works. Thus, despite these limitations, the present investigation achieved its goals.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising one or more ruthenium (Ru)-based agents, wherein in vitro or in vivo exposure of the composition to a biological sample results in inhibition of venom related procoagulant activity, inhibition of venom related PLA₂ activity, and/or inhibition of venom related thrombus generation.

2. The composition of claim 1, wherein the one or more ruthenium (Ru)-based agents is a ruthenium compound selected from zerovalent, divalent and trivalent ruthenium compounds.

3. (canceled)

4. The composition of claim 2, wherein the ruthenium compound is selected from ruthenium hexafluoride, Ruthenium(IV) Oxide, Ruthenium(VIII) Oxide, Ruthenium(VIII) Oxide, Ruthenium(III) Nitrate, Ruthenium(III) Phosphate, Ruthenium(IV) Sulfate, Ruthenium(II) Nitrate, Ruthenium(IV) Sulfite, Ruthenium(III) Fluoride, Ruthenium(II) Perchlorate, Ruthenium(VI) Sulfide, Ruthenium(III) Nitride, Ruthenium(III) Iodide, Ruthenium Phosphide, Ruthenium(IV) Metasilicate, Ruthenium(III) Acetate, Ruthenium boride, Strontium ruthenate, Lithium ruthenate, Tetrapropylammonium perruthenate, Diruthenium tetraacetate chloride, Uranium ruthenium silicide, Ruthenium hexafluoride, Ruthenium pentafluoride, Cis-Dichlorobis(bipyridine)ruthenium(II), Dicarbonyltris(triphenylphosphine)ruthenium(0), Ruthenium anti-cancer drugs (e.g., KP1019, NAMI-A, Pentaamine(dinitrogen)ruthenium(II) chloride, RAPTA), Ru360 (e.g., an oxo-bridged dinuclear ruthenium ammine complex with an absorption spectrum maximum at 360 nm), Ruthenium red, Ruthenium(III) acetylacetonate, Ruthenium diamine, (Terpyridine)ruthenium trichloride, Tetrasodium tris(bathophenanthroline disulfonate)ruthenium(II), Tris(bipyridine)ruthenium(II) chloride, triruthenium(0) dodecacarbonyl, dichloro(benzene)ruthenium(II) dimer, dichloro(p-cymene)ruthenium(II) dimer, dichloro(mesitylene)ruthenium(II) dimer, dichloro(hexamethylbenzene)ruthenium(II) dimer, diiodo(p-cymene)ruthenium(II) dimer, dipivalato(p-cymene)ruthenium(II), bis(.pi.-methallyl)(1,5-cyclooctadiene)ruthenium(II), dichloro(1,5-cyclooctadiene)ruthenium(II) polymer, dichloro(norbornadiene)ruthenium(II) polymer, dichlorotris(triphenylphosphine)ruthenium(II), chlorohydridotris(triphenylphosphine)ruthenium(II) toluene adduct, dihydridotetrakis(triphenylphosphine)ruthenium(II), carbonylchlorohydridotris(triphenylphosphine)ruthenium(II), carbonyldihydridotris(triphenylphosphine)ruthenium(II), dichlorotetrakis(dimethylsulfoxide)ruthenium(II), ruthenium(III) chloride, ruthenium(III) chloride hydrate, ruthenium(III) iodide, ruthenium(III) iodide hydrate, hexaammineruthenium(III) trichloride, and ruthenium(III) acetylacetonate

5. The composition of claim 2, wherein the ruthenium compound is a ruthenium halide (e.g., RuCl₃, RuCl₃·H₂O, RuI₃ and hydrated RuBr₃).

6. The composition of claim 2, wherein the ruthenium compound has at least one at least one tertiary phosphine ligand (e.g., Ru(CO)₃(PPh₃)₂, RuCl₂(CO)₂(PPh₃)₂, RuCl₂(PPh₃)₄, RuH₂(PPh₃)₄, Ru(CH₂═CH₂)(PPh₃)₃, RuHCl(PPh₃)₃·C₇H₈ complex and RuHCl(PPh₃)₃).

7. The composition of claim 1, wherein the one or more Ru-based agents comprise a Ru-based radical and/or Ru-based ion.

8. The composition of claim 1, wherein the one or more Ru-based agents comprise a Ru-based radical intermediate formed during carbon monoxide release from any Ru-based carbon-monoxide releasing molecule (e.g., tricarbonyldichlororuthenium(II) dimer (CORM-2) and tricarbonylchloro(glycinato)ruthenium (CORM-3).

9. The composition of claim 1, wherein the one or more Ru-based agents comprise a combination of agents having varying valences.

10. The composition of claim 1, wherein the agents having varying valences comprises a first agent having a valence of two, and a second agent having a valence of three.

11. The composition of claim 10, wherein the first agent having a valence of two is selected from tricarbonyldichlororuthenium(II) dimer (CORM-2) and tricarbonylchloro(glycinato)ruthenium (CORM-3).

12. The composition of claim 10, wherein the second agent having a valence of three is selected from RuCl₃ (Ru(III), New Anticancer Metastasis Inhibitor (NAMI-A), and trans-[tetrachlorobis(1H-indazole)ruthenate(III) (KP1019).

13. The composition of claim 1, wherein the composition comprises a combination of CORM-2 and RuCl₃.

14. The composition of claim 1, wherein the composition is a pharmaceutical composition.

15. The composition of claim 10, wherein the amounts of the first agent having a valence of two, and a second agent having a valence of three within the composition is such that upon administration to a subject (e.g., a human subject), the composition is able to treat, ameliorate and/or prevent the toxic effects of venom poisoning.

16. The composition of claim 10, wherein the amounts of the first agent having a valence of two, and a second agent having a valence of three within the composition is such that upon administration to a subject (e.g., a human subject), the composition is able to prevent one or more of
venom mediated catalysis of fibrinogen in the subject,
venom mediated degradation of plasma coagulation in the subject,
venom mediated coagulopathy in the subject, and
venom mediated catalysis and inactivation of fibrinogen.

17-20. (canceled)

21. A method for enhancing coagulation or reducing fibrinolysis in a subject (e.g., a human subject) suffering from or at risk of suffering from venom poisoning, comprising administering to the subject a composition as described in claim 1, wherein the administering results in prevention of one or more of venom mediated catalysis of fibrinogen in the subject, venom mediated degradation of plasma coagulation in the subject, venom mediated coagulopathy in the subject, and venom mediated catalysis and inactivation of fibrinogen.

22. The method of claim 21, wherein the venom is from one of the following: Bothrops oxyranus, Calloselasma, P. textils, Echis, Crotalus, P. textilis, Naja naja (Indian cobra), Bothrops asper (Fur-de-lance), Agkistrodon piscivorus piscivorus, Agkistrodon contortrix contortrix, Agkistrodon contortrix laticinctus, Askistrodon contortix pictigaster, Agkistrodon piscivorus leucostoma, Agkistrodon contortrix mokasen, Northern Pacific rattlesnake, Arizona Black rattlesnake, Prairie rattlesnake, Red Diamond rattlesnake, Timber rattlesnake, Eastern Diamondback rattlesnake, and Southern Pacific rattlesnake.

23. The method of claim 21, wherein the administering results in inhibition of venom related procoagulant activity, inhibition of venom related PLA₂ activity, and/or inhibition of venom related thrombus generation.

24-29. (canceled)

30. A kit comprising a composition of claim 1, an antivenom composition, and instructions for administering the composition to a living mammal.

31. The kit of claim 30, further comprising one or more of a hemostatic agent, a coagulant, an anti-fibrinolytic medication, a blood coagulation factor, fibrin, thrombin, recombinant activated factor VII, prothrombin complex concentrate, FEIBA, or a therapeutic agent selected from the group consisting of an antibiotic, an anesthetic, an analgesic, an antihistamine, an antimicrobial, an antifungal, an antiviral, and an anti-inflammatory agent.
